(19) United States
(12) Patent Application Publication
(10) Pub. No.: US 2025/0382636 A1
(43) Pub. Date: Dec. 18, 2025

(54) COMPOSITIONS FOR MAINTAINING LENTIVIRAL VECTOR AND USES THEREOF

(71) Applicant: Regeneron Pharmaceuticals, Inc., Tarrytown, NY (US)

(72) Inventors: Moumita Bhattacharya, Andover, MA (US); Kristen Kelly, Pembroke, MA (US)

(21) Appl. No.: 18/867,487

(22) PCT Filed: May 24, 2023
(86) PCT No.: PCT/US2023/067406

§ 371 Date: Nov. 20, 2024

Related U.S. Application Data
(60) Provisional application No. 63/346,001, filed on May 26, 2022.

(51) Int. Cl. C12N 15/86

(52) U.S. Cl. CPC C12N 15/86; C12N 2740/16043; C12N 2740/16051

(57) ABSTRACT

The present disclosure provides improved aqueous formulations for storing viral vectors and methods of preparing and using the same.

### RELATED APPLICATIONS

The instant application claims priority to U.S. Provisional Application No. 63/346,001 filed on May 26, 2022. The entire contents of the foregoing application are expressly incorporated by reference herein.

### BACKGROUND

### Technical Field

The present disclosure relates to viral vector compositions having improved stability at room, refrigerated, and frozen temperatures. More particularly, the disclosure relates to improved aqueous and frozen liquid viral vector compositions which display high titer recovery, particle integrity, and potency after storage in various conditions.

### Description of the Related Art

Viral vectors have emerged as a prominent way to deliver therapeutic cargo to target cells. Such vectors include systems derived from adenovirus, adeno-associated virus (AAV), herpes simplex virus (HSV), and lentivirus. These vectors are capable of introducing and/or integrating a gene of interest (e.g., a therapeutic gene) into the target cell's genome. Thus, a target cell can be theoretically edited to express any gene of interest, which may also have a therapeutic benefit. Such therapies are already in the clinic (see, e.g., Curr Gene Ther 2015; 15 (1): 64-81 and Blood Rev. 2022 Jan. 21; 100929) and represent a transformative approach to treating certain diseases.

One factor limiting the use of viral vectors in a clinical setting (including lentivirus) is low stability, aggregation, and loss of viral titer after storage at temperatures above and below freezing (e.g., see, Kumru et al., J Pharm Sci. 2018 November; 107 (11): 2764-2774). Indeed, viral vectors may be subjected to a wide range of temperatures during manufacturing, storage, and eventual research or commercial use. The viral vectors may be subjected to physiological temperatures during manufacturing and transduction (e.g., 25° C. and/or 37° C.), cooler temperatures during purification and short-term storage (e.g., 2-8° C.), and freezing temperatures (e.g., 0° C. or lower) during long term storage. Moreover, viral vectors may undergo several freeze-thaws throughout their useful life and storage.

Accordingly, there remains a need for improved compositions for long-term storage of viral vectors.

### BRIEF SUMMARY

The present disclosure generally relates, in part, to improved compositions for storing viral vectors including but not limited to retroviral or lentiviral vectors.

In one aspect, an aqueous viral composition is provided, comprising a viral vector, a HEPES or L-Histidine buffer, a carbohydrate, and an amino acid.

In another aspect, an aqueous viral composition is provided, wherein the composition comprises a viral vector; a HEPES buffer; a carbohydrate; a salt; and a poloxamer.

In various embodiments, the buffer is present at a concentration of about 25 mM to about 30 mM, about 26 mM to about 29 mM, about 27 mM to about 28 mM, or about 27.5 mM. In particular embodiments, the buffer is a HEPES buffer. In particular embodiments, the buffer is an L-Histidine buffer.

In various embodiments, the carbohydrate is present at a concentration of about 66 mM to about 80 mM, about 67 mM to about 79 mM, about 68 mM to about 78 mM, about 69 mM to about 77 mM, about 70 mM to about 76 mM, about 71 mM to about 75 mM, about 72 mM to about 74 mM, or about 73 mM. In various embodiments, the carbohydrate is present at a concentration of about 2.0% to about 3.0% by weight per volume of the composition, about 2.1% to about 2.9% by weight per volume of the composition, about 2.2% to about 2.8% by weight per volume of the composition, about 2.3% to about 2.7% by weight per volume of the composition, about 2.4% to about 2.6% by weight per volume of the composition, or about 2.5% by weight per volume of the composition. In various embodiments, the carbohydrate is a disaccharide. In various embodiments, the carbohydrate is lactose, glucose, mannose, mannitol, sorbitol, sucrose, trehalose, and/or glycerol. In some embodiments, the carbohydrate is sucrose and/or trehalose. In particular embodiments, the carbohydrate is sucrose. In particular embodiments, the carbohydrate is trehalose.

In various embodiments, the amino acid is present at a concentration of about 40 mM to about 60 mM, about 41 mM to about 59 mM, about 42 mM to about 58 mM, about 43 mM to about 57 mM, about 44 mM to about 56 mM, about 45 mM to about 55 mM, about 46 mM to about 54 mM, about 47 mM to about 53 mM, about 48 mM to about 52 mM, about 49 mM to about 51 mM, or about 50 mM. In some embodiments, the amino acid is a non-polar amino acid. In some embodiments, the amino acid is selected from the group consisting of: glycine, alanine, valine, leucine, methionine, isoleucine phenylalanine, tyrosine, and tryptophan. In some embodiments, the amino acid is selected from the group consisting of: phenylalanine, tyrosine, tryptophan, and proline. In particular embodiments, the amino acid is L-Proline.

In various embodiments, the composition further comprises a salt. In some embodiments, the salt is present at a concentration of about 65 mM to about 85 mM, about 66 mM to about 84 mM, about 67 mM to about 83 mM, about 68 mM to about 82 mM, about 69 mM to about 81 mM, about 70 mM to about 80 mM, about 71 mM to about 79 mM, about 72 mM to about 78 mM, about 73 mM to about 77 mM, about 74 mM to about 76 mM, about 75 mM. In some embodiments, the salt is a chloride salt, KCl, or NaCl. In particular embodiments, the salt is NaCl.

In various embodiments, the composition further comprises a poloxamer. In some embodiments, the poloxamer is present at a concentration of about 0.01 mg/ml to about 1 mg/ml, about 0.02 mg/ml to about 0.9 mg/ml, about 0.03 mg/ml to about 0.8 mg/ml, about 0.04 mg/ml to about 0.7 mg/ml, about 0.05 mg/ml to about 0.6 mg/ml, about 0.06 mg/ml to about 0.5 mg/ml, about 0.07 mg/ml to about 0.4 mg/ml, about 0.08 mg/ml to about 0.3 mg/ml, about 0.09 mg/ml to about 0.2 mg/ml, about 0.1 mg/ml to about 0.8 mg/ml, about 0.1 mg/ml to about 0.5 mg/ml, or about 0.2 mg/ml to about 0.4 mg/ml. In certain embodiments, the poloxamer is present at a concentration of about 0.1 mg/ml. In certain embodiments, the poloxamer is present at a concentration of about 0.3 mg/ml. In some embodiments, the poloxamer is poloxamer 188, poloxamer 288, poloxamer 335, poloxamer 338, or poloxamer 407. In particular embodiments, the poloxamer is poloxamer 188 (P188).

In various embodiments, the composition comprises a pH of about 6.5 to about 8. In some embodiments, the composition comprises a pH of about 6.5. In some embodiments, the composition comprises a pH of about 7. In some embodiments, the composition comprises a pH of about 7.5. In some embodiments, the composition comprises a pH of about 8.

In a particular embodiment, an aqueous viral composition is provided, wherein the composition comprises a viral vector; about 27.5 mM HEPES; about 73 mM sucrose or about 2.5% sucrose by weight per volume of composition; and about 50 mM L-Proline; wherein the composition comprises a pH of about 7.

In a particular embodiment, an aqueous viral composition is provided, wherein the composition comprises a viral vector; about 27.5 mM L-Histidine; about 73 mM sucrose or about 2.5% sucrose by weight per volume of composition; and about 50 mM L-Proline; wherein the composition comprises a pH of about 7.

In a particular embodiment, an aqueous viral composition is provided, wherein the composition comprises a viral vector; about 27.5 mM HEPES; about 73 mM sucrose or about 2.5% sucrose by weight per volume of composition; about 50 mM L-Proline; and about 0.1 to about 0.8 mg/mL poloxamer 188; wherein the composition comprises a pH of about 7.

In a particular embodiment, an aqueous viral composition is provided, wherein the composition comprises a viral vector; about 27.5 mM HEPES; about 73 mM sucrose or about 2.5% sucrose by weight per volume of composition; about 50 mM L-Proline; and about 0.3 mg/mL poloxamer 188; wherein the composition comprises a pH of about 7.

In a particular embodiment, an aqueous viral composition is provided, wherein the composition comprises a viral vector; about 27.5 mM HEPES; about 73 mM sucrose or about 2.5% sucrose by weight per volume of composition; about 50 mM L-Proline; and about 75 mM NaCl; wherein the composition comprises a pH of about 7.

In a particular embodiment, an aqueous viral composition is provided, wherein the composition comprises a viral vector; about 27.5 mM HEPES; about 73 mM sucrose or about 2.5% sucrose by weight per volume of composition; about 50 mM L-Proline; about 75 mM NaCl; and about 0.1 to about 0.8 mg/mL poloxamer 188; wherein the composition comprises a pH of about 7.

In various embodiments, the composition further comprises a salt. In some embodiments, the salt is present at a concentration of about 65 mM to about 85 mM, about 66 mM to about 84 mM, about 67 mM to about 83 mM, about 68 mM to about 82 mM, about 69 mM to about 81 mM, about 70 mM to about 80 mM, about 71 mM to about 79 mM, about 72 mM to about 78 mM, about 73 mM to about 77 mM, about 74 mM to about 76 mM, about 75 mM. In particular embodiments, the salt is NaCl.

In a particular embodiment, an aqueous viral composition is provided, wherein the composition comprises a viral vector; about 27.5 mM HEPES; about 2.5% sucrose by weight per volume of composition; about 75 mM NaCl; and about 0.1 to about 0.8 mg/ml poloxamer 188 or about 0.01% to about 0.08% poloxamer 188 by weight per volume of composition; wherein the composition comprises a pH of about 7.

In any of the aspects and embodiments contemplated herein, the viral vector is present at a titer from about 1×10⁸ to about 2×10⁹ TU/ml. In various embodiments, the viral vector is present at a titer of about 1×10⁸ TU/ml, about 2×10⁸ TU/ml, about 3×10⁸ TU/ml, about 4×10⁸ TU/ml, about 5×10⁸ TU/ml, about 6×10⁸ TU/ml, about 7×10⁸ TU/ml, about 8×10⁸ TU/ml, about 9×10⁸ TU/ml, about 1×10⁹ TU/ml, or about 2×10⁹ TU/ml.

In various embodiments, the vector is an adenoviral vector, an adeno-associated viral (AAV) vector, a herpes virus vector, a vaccinia virus vector, or a retroviral vector. In some embodiments, the viral vector is a lentiviral vector. In some embodiments, the lentiviral vector is selected from the group consisting of: human immunodeficiency virus 1 (HIV-1); human immunodeficiency virus 2 (HIV-2), visna-maedi virus (VMV) virus; caprine arthritis-encephalitis virus (CAEV); equine infectious anemia virus (EIAV); feline immunodeficiency virus (FIV); bovine immune deficiency virus (BIV); and simian immunodeficiency virus (SIV). In some embodiments, the lentiviral vector is derived from human immunodeficiency cirus-1 (HIV-1) or human immunodeficiency virus 2 (HIV-2). In particular embodiments, the lentiviral vector is derived from human immunodeficiency cirus-1 (HIV-1).

In any of the aspects and embodiments contemplated herein, the viral vector is pseudotyped. In various embodiments, the viral vector is pseudotyped with an envelope protein from a strain of vesicular stomatitis virus. In some embodiments, the strain of vesicular stomatitis virus is selected from the group consisting of: Indiana, Alagoas, New Jersey, Isfahan, CoCal, Maraba, or Piry. In particular embodiments, the viral vector is pseudotyped with a vesicular stomatitis virus G (VSV-G) protein. In some embodiments, the viral vector is pseudotyped with an envelope derived from a Measles envelope protein, a Sindbis envelope protein, Morbillivirus F and H proteins, Sendai F and HN proteins, or Paramyxoviridae F and H proteins.

In any of the aspects and embodiments contemplated herein, the viral vector comprises a polynucleotide comprising a transgene. In some embodiments, the transgene encodes a therapeutic protein. In some embodiments, the transgene or therapeutic protein is a chimeric antigen receptor (CAR), a chimeric costimulatory receptor (CCR), an aβ T cell receptor (aβ-TCR), a γδ T cell receptor (γδ-TCR), a dimerizing agent regulated immunoreceptor complex (DARIC), or switch receptor. In some embodiments, the transgene or therapeutic protein is a for the treatment of a monogenetic disease, disorder, or condition. In some embodiments, the transgene or therapeutic protein is a therapeutic globin for treatment of a hemoglobinopathy or an ABCD1 gene for the treatment of CALD.

In particular embodiments, the compositions contemplated herein do not comprise serum, human serum albumin (HSA), PIPES, sodium citrate, sodium phosphate, and/or Tris. In some embodiments, the compositions contemplated herein do not comprise salt. In some embodiments, the compositions contemplated herein do not comprise NaCl. In some embodiments, the compositions contemplated herein do not comprise KCl. In some embodiments, the compositions contemplated herein do not comprise trehalose.

In any of the aspects and embodiments contemplated herein, the viral vector maintains greater than about 75%, about 80%, about 85%, about 90%, or about 95% infectious titer recovery in HOS cells after storage at 25° C. for 24 hours, storage at 2-8° C. for 48 hours, storage at 2-8° C. for 168 hours, and/or at least 1 freeze-thaw cycle; relative to the infectious titer of the viral vector in the composition prior to storage or at least one freeze-thaw cycle.

In any of the aspects and embodiments contemplated herein, the viral vectors have a thermal unfolding temperature of about 56° to about 62° C., as measured by differential scanning fluorimetry (DSF).

In any of the aspects and embodiments contemplated herein, the viral vectors have a thermal unfolding temperature of about 58° to about 60° C., as measured by differential scanning fluorimetry (DSF).

In any of the aspects and embodiments contemplated herein, the viral vectors maintain a hydrodynamic diameter of about 150 nm to about 170 nm as measured by dynamic light scattering (DLS) at 25° C. and a viscosity value of 0.967 centipoise (cP), relative to hydrodynamic diameter of the viral vector in the composition prior to storage or at least one freeze-thaw cycle.

In any of the aspects and embodiments contemplated herein, the viral vectors maintain at least 78%, at least 80%, at least 85%, at least 90%, or at least 95% potency as measured by transgene expression in PBMCs compared to a reference standard, after storage.

In any of the aspects and embodiments contemplated herein, the composition has no visible fiber particles (wispy fibers) after storage. In any of the aspects and embodiments contemplated herein, the composition has 5 or fewer visible particles or specs after storage. In any of the aspects and embodiments contemplated herein, the composition has 4 or fewer visible particles or specs after storage. In any of the aspects and embodiments contemplated herein, the composition has 3 or fewer visible particles or specs after storage. In any of the aspects and embodiments contemplated herein, the composition has 2 or fewer visible particles or specs after storage. In any of the aspects and embodiments contemplated herein, the composition has 1 or fewer visible particles or specs after storage. In any of the aspects and embodiments contemplated herein, the composition has no visible particles after storage.

In various embodiments, the storage is at 25° C., 2-8° C., or 37° C. In some embodiments, the storage is for 24 hours, 48 hours, or 72, 96, 120, 144, or 168 hours, or more.

In various embodiments, the storage comprises one or more freeze-thaw cycles. In some embodiments, the one or more freeze-thaw cycle is 1, 2, 3, 4, or 5 freeze thaw cycles. In some embodiments, the one or more freeze-thaw cycles comprise freezing the composition at about −65° or less for about 1.5 hours or more, and thawing at 30° C. for 1.5 hours.

In any of the aspects and embodiments contemplated herein, the composition is frozen.

In another aspect, a method for storing a viral vector is provided, comprising providing a viral vector, contacting the viral vector with any of the compositions contemplated herein, and storing the viral composition at a temperature of about 25° C. or lower.

In another aspect, a method for storing a viral vector is provided, comprising providing a viral vector, contacting the viral vector with any of the compositions contemplated herein, and storing the viral composition at a temperature of about 2-8° C. or lower for at least about 24 hours.

In another aspect, a method for cryopreserving a viral vector is provided, comprising providing a viral vector, contacting the viral vector with any one of the compositions contemplated herein, freezing the viral composition, and storing the viral composition at a temperature of about 0° C. or lower.

In various embodiments, the methods contemplated herein comprise storing the viral composition for at least about 24 hours, 48 hours, at least about 72 hours, at least about 96 ours, at least about 120 hours, at least about 148 hours, or at least about 168 hours.

In another aspect, a method of expressing a transgene in a cell is provided, comprising contacting a cell with any one of the compositions contemplated herein. In various embodiments the cell is a mammalian cell. In some embodiments, the cell is a hematopoietic cell. In some embodiments, the cell is a hematopoietic stem or progenitor cell. In some embodiments, the cell is a human CD34⁺ hematopoietic or progenitor cell. In some embodiments, the cell is a T cell. In some embodiments, the cell is an aβ T cell. In some embodiments, the cell is a γδ T cell. In some embodiments, the cell is a CD3⁺, CD4⁺, and/or CD8⁺ cell. In some embodiments, the cell is an immune effector cell. In some embodiments, the cell is a cytotoxic T lymphocyte (CTL), a tumor infiltrating lymphocyte (TIL), or a helper T cell. In some embodiments, the cell is a natural killer (NK) cell or natural killer T (NKT) cell.

### BRIEF DESCRIPTION OF SEVERAL VIEWS OF THE DRAWINGS

FIG. 1 shows an illustration of the screening process/steps and formula components tested during the screening process.

FIG. 2 shows an illustration of the process flow diagram, conditions tested, and quality assessment techniques/assays.

FIG. 3 shows infectious titer and percent (%) recovery of various lentiviral vector compositions after storage at 25° C. for 24 hours or 2-8° C. for 48 hours.

FIG. 4 shows hydrodynamic size of lentiviral vector particles suspended in various compositions after storage at 37° C. for 48 hours, 25° C. for 24 hours, or 2-8° C. for 48 hours.

FIG. 5 shows the thermal unfolding of lentiviral vector compositions over the range of 20-90° C.

FIG. 6 shows infectious titer and percent (%) recovery of various lentiviral vector compositions after storage at 25° C. for 24 hours or 2-8° C. for 48 hours.

FIG. 7 shows hydrodynamic size of lentiviral vector particles suspended in various compositions after storage at 37° C. for 48 hours, 25° C. for 24 hours, or 2-8° C. for 48 hours.

FIG. 8 shows the thermal unfolding of lentiviral vector compositions over the range of 20-90° C.

FIG. 9 shows infectious titer and percent (%) recovery of various lentiviral vector compositions after storage at 25° C. for 24 hours or 2-8° C. for 48 hours.

FIG. 10 shows infectious titer and percent (%) recovery of various lentiviral vector compositions after storage at 25° C. for 24 hours or 2-8° C. for 48 hours.

FIG. 11 shows infectious titer and percent (%) recovery of various lentiviral vector compositions after 5 freeze-thaw cycles.

FIG. 12 shows hydrodynamic size of lentiviral vector particles suspended in various compositions after storage at 37° C. for 48 hours, 25° C. for 24 hours, or 2-8° C. for 48 hours.

FIG. 13 shows hydrodynamic size of lentiviral vector particles suspended in various compositions after 5 freeze-thaw cycles.

FIG. 14 shows infectious titer and percent (%) recovery of various HEPES lentiviral vector compositions after storage at 25° C. for 24 hours or 2-8° C. for 168 hours.

FIG. 15 shows hydrodynamic size of lentiviral vector particles suspended in various HEPES compositions after storage at 2-8° C. for 168 hours.

FIG. 16 shows a visible particle assessment of various HEPES lentiviral compositions after storage at 2-8° C. for 48, 96, or 168 hours.

FIG. 17 shows infectious titer and percent (%) recovery of various HEPES lentiviral vector compositions after 5 freeze-thaw cycle.

FIG. 18 shows hydrodynamic size of lentiviral vector particles suspended in various HEPES compositions after 5 freeze-thaw cycles.

FIG. 19 shows a visible particle assessment of various HEPES lentiviral compositions after 5 freeze-thaw cycles.

FIG. 20 shows infectious titer and percent (%) recovery of various L-Histidine lentiviral vector compositions after storage at 25° C. for 24 hours or 2-8° C. for 168 hours.

FIG. 21 shows hydrodynamic size of lentiviral vector particles suspended in various L-Histidine compositions after storage at 2-8° C. for 168 hours.

FIG. 22 shows a visible particle assessment of various L-Histidine lentiviral compositions after storage at 2-8° C. for 48, 96, or 168 hours.

FIG. 23 shows infectious titer and percent (%) recovery of various L-Histidine lentiviral vector compositions after 5 freeze-thaw cycles.

FIG. 24 shows hydrodynamic size of lentiviral vector particles suspended in various L-Histidine compositions after 5 freeze-thaw cycles.

FIG. 25 shows a visible particle assessment of various L-Histidine lentiviral compositions after 5 freeze-thaw cycles.

FIG. 26 shows intermediate bulk titer recovery of different lentiviral vectors after storage at 2-8° C. for 24, 120, or 168 hours.

FIG. 27 shows a visible particle assessment of various lentiviral compositions with and without P188 after manufacturing.

FIG. 28 shows a visible particle formation of lentiviral compositions with and without P188.

FIG. 29 shows recovery and infectious titer of different batches of lentiviral vectors formulated with and without P188 after storage at 2-8° C. for 24, 120, or 168 hours.

FIG. 30 shows infectious titer of lentiviral compositions with and without P188 after 6-months storage at ≤−65° C.

FIG. 31 shows a relative potency assessment of lentiviral compositions.

### DETAILED DESCRIPTION

### A. Overview

The present disclosure generally relates to, in part, viral vector (e.g., lentiviral vector) compositions which display high titer recovery, thermostability, particle integrity, no to low visible particle formation, and potency after storage in various conditions. Without wishing to be bound by any particular theory, many biologics (including viruses) lose protein structure and activity during the manufacturing process and storage if optimal solutions or formulations have not been determined. Viral vectors also lose their ability to efficiently transduce cells and change in structure when undergoing similar manufacturing and storage stresses in various solutions.

Thus, contemplated herein are formulations that surprisingly minimize viral vector loss, and maintain viral vector integrity and activity in conditions that are known to impact virus integrity. Specifically, the formulations contemplated herein demonstrate high titer recovery, particle integrity, thermal stability, no, to low visible particle formation (e.g., only 1, 2, or 3 visible particles), and potency after storage in various conditions, including storage at 37°, 25° C., 2-8° C., and/or after one or more freeze-thaw cycles. Also contemplated are methods of storage, cryopreservation, and transduction.

Techniques for recombinant (i.e., engineered) DNA, peptide and oligonucleotide synthesis, immunoassays, tissue culture, transformation (e.g., electroporation, lipofection), enzymatic reactions, purification and related techniques and procedures may be generally performed as described in various general and more specific references in microbiology, molecular biology, biochemistry, molecular genetics, cell biology, virology and immunology as cited and discussed throughout the present specification. See, e.g., Sambrook et al., Molecular Cloning: A Laboratory Manual, 3d ed., Cold Spring Harbor Laboratory Press, Cold Spring Harbor, N. Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid The Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3rd Edition, 2010 Humana Press); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir and CC Blackwell, eds., 1986); Roitt, Essential Immunology, 6th Edition, (Blackwell Scientific Publications, Oxford, 1988); Current Protocols in Immunology (Q. E. Coligan, A. M. Kruisbeek, D. H. Margulies, E. M. Shevach and W. Strober, eds., 1991); Annual Review of Immunology; as well as monographs in journals such as Advances in Immunology.

### B. Definitions

Prior to setting forth this disclosure in more detail, it may be helpful to an understanding thereof to provide definitions of certain terms to be used herein.

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length=15%, +10%, +9%, +8%, +7%, +6%, +5%, +4%, +3%, +2%, or +1% about a reference quantity, level, value, number, frequency, percentage, dimension, size, amount, weight or length. For example, a composition having a pH of “about 7” means the composition has a pH of 7±1%-15% (e.g., ±15%, ±10%, ±9%, ±8%, ±7%, ±6%, ±5%, ±4%, ±3%, ±2%, or ±1%).

In one embodiment, a range, e.g., 1 to 5, about 1 to 5, or about 1 to about 5, refers to each numerical value encompassed by the range. For example, in one non-limiting and merely illustrative embodiment, the range “1 to 5” is equivalent to the expression 1, 2, 3, 4, 5; or 1.0, 1.5, 2.0, 2.5, 3.0, 3.5, 4.0, 4.5, or 5.0; or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

As used herein, the term “buffer” refers to a mixture of a weak acid and its conjugate base or a weak base and its conjugate acid.

As used herein, the term “freeze-thaw” or “freeze-thaw cycle” refers to exposure of a liquid mixture, such as an aqueous solution or suspension, to a temperature at or less than its freezing point until the mixture is frozen, followed by thawing the mixture at a temperature greater than its freezing point. The freezing step can be performed, e.g., by placing the mixture in an environment in which the temperature is from about 0° C. to about −80° C. In some embodiments, the freezing temperature is from about-20° C. to about-80° C. In some embodiments, the freezing temperature is about-65° C. The frozen aqueous solution or suspension may be stored for a period of one or more hours, days, weeks, months, or years prior to thawing. Thawing of the solution or suspension can be performed by exposing it to conditions in which the temperature is from about 2° C. to about 8° C., or by storing the mixture at room temperature (e.g., about 25° C.). In some embodiments, the solution or suspension is thawed at 30° C. In some embodiments, the solution or suspension is thawed at 37° C.

As used herein, the term “percent by weight per volume” or “% w/v” denotes the percentage weight (in grams) of a single component relative to the total volume of the mixture that contains the component. For instance, 500 mg of a component in a total volume of 8 ml is 6.25% w/v, and 500 mg of a component in a total volume of 5 ml is 10% w/v.

As used herein, the term “titer” or “viral titer” refers to the number of infectious vector particles, or “transducing units,” that result in the production of a trans gene product in a target cell. Viral titer can be measured by a functional assay, such as an assay described in Xiao et al., Exp. Neurobiol. 144:113-124, 1997; Fisher et al., J. Viral. 70:520-532, 1996; and GENE Therapy (2002), 9, 1155-1162; each of which are herein incorporated by reference in their entirety. Alternatively, viral titer can be measured by determining the quantity of viral DNA that has integrated into a host cell genome, e.g., using polymerase chain reaction (PCR) techniques known in the art.

Additional definitions are set forth throughout this disclosure.

### C. Formulations

Disclosed herein are formulations that surprisingly minimize viral vector loss and maintain activity in conditions that are known to impact virus integrity. The formulations contemplated herein comprise at least a buffer and a carbohydrate.

In certain embodiments, the buffer is an N-(2-Hydroxyethyl) piperazine-N′-(2-ethanesulfonic acid) (HEPES) buffer. The chemical formula of HEPES is C₈H₁₈N₂O₄S and it has a molecular weight of 238.3012 g/mol. The IUPAC ID for HEPES is 2-[4-(2-hydroxyethyl) piperazin-1-yl]ethanesulfonic acid.

In various embodiments, the buffer is an L-Histidine buffer. L-Histidine is the L-enantiomer of the amino acid histidine. L-Histidine has a chemical formula of C₆H₉N₃O₂ and a molecular weight of 155.157 g/mol.

In some embodiments, the buffer is present at a concentration of about 25 mM to about 30 mM. In some embodiments, the buffer is present at a concentration of about 26 mM to about 29 mM. In some embodiments, the buffer is present at a concentration of about 27 mM to about 28 mM.

In some embodiments, the buffer is present at a concentration of about 25 mM. In some embodiments, the buffer is present at a concentration of about 25.5 mM. In some embodiments, the buffer is present at a concentration of about 26 mM. In some embodiments, the buffer is present at a concentration of about 26.5 mM. In some embodiments, the buffer is present at a concentration of about 27 mM. In some embodiments, the buffer is present at a concentration of about 27.5 mM. In certain embodiments, the buffer is present at a concentration of 27.5 mM. In some embodiments, the buffer is present at a concentration of about 28 mM. In some embodiments, the buffer is present at a concentration of about 28.5 mM. In some embodiments, the buffer is present at a concentration of about 29 mM. In some embodiments, the buffer is present at a concentration of about 29.5 mM. In some embodiments, the buffer is present at a concentration of about 30 mM.

In various embodiments, the carbohydrate is present at a concentration of about 66 mM to about 80 mM. In some embodiments, the carbohydrate is present at a concentration of about 67 mM to about 79 mM. In some embodiments, the carbohydrate is present at a concentration of about 68 mM to about 78 mM. In some embodiments, the carbohydrate is present at a concentration of about 69 mM to about 77 mM. In some embodiments, the carbohydrate is present at a concentration of about 70 mM to about 76 mM. In some embodiments, the carbohydrate is present at a concentration of about 71 mM to about 75 mM. In some embodiments, the carbohydrate is present at a concentration of about 72 mM to about 74 mM.

In some embodiments, the carbohydrate is present at a concentration of about 66 mM. In some embodiments, the carbohydrate is present at a concentration of about 67 mM. In some embodiments, the carbohydrate is present at a concentration of about 68 mM. In some embodiments, the carbohydrate is present at a concentration of about 69 mM. In some embodiments, the carbohydrate is present at a concentration of about 70 mM. In some embodiments, the carbohydrate is present at a concentration of about 71 mM. In some embodiments, the carbohydrate is present at a concentration of about 72 mM. In some embodiments, the carbohydrate is present at a concentration of about 73 mM. In certain embodiments, the carbohydrate is present at a concentration of 73 mM. In some embodiments, the carbohydrate is present at a concentration of about 74 mM. In some embodiments, the carbohydrate is present at a concentration of about 75 mM. In some embodiments, the carbohydrate is present at a concentration of about 76 mM. In some embodiments, the carbohydrate is present at a concentration of about 77 mM. In some embodiments, the carbohydrate is present at a concentration of about 78 mM. In some embodiments, the carbohydrate is present at a concentration of about 79 mM. In some embodiments, the carbohydrate is present at a concentration of about 80 mM.

In various embodiments, the carbohydrate is present at a concentration of about 15 mM to about 45 mM. In some embodiments, the carbohydrate is present at a concentration of about 20 mM to about 40 mM. In some embodiments, the carbohydrate is present at a concentration of about 25 mM to about 35 mM. In some embodiments, the carbohydrate is present at a concentration of about 25 mM to about 34 mM. In some embodiments, the carbohydrate is present at a concentration of about 25 mM to about 33 mM. In some embodiments, the carbohydrate is present at a concentration of about 26 mM to about 32 mM. In some embodiments, the carbohydrate is present at a concentration of about 27 mM to about 31 mM. In some embodiments, the carbohydrate is present at a concentration of about 28 mM to about 30 mM.

In some embodiments, the carbohydrate is present at a concentration of about 15 mM. In some embodiments, the carbohydrate is present at a concentration of about 16 mM. In some embodiments, the carbohydrate is present at a concentration of about 17 mM. In some embodiments, the carbohydrate is present at a concentration of about 18 mM. In some embodiments, the carbohydrate is present at a concentration of about 19 mM. In some embodiments, the carbohydrate is present at a concentration of about 20 mM. In some embodiments, the carbohydrate is present at a concentration of about 21 mM. In some embodiments, the carbohydrate is present at a concentration of about 22 mM. In some embodiments, the carbohydrate is present at a concentration of about 23 mM. In some embodiments, the carbohydrate is present at a concentration of about 24 mM. In some embodiments, the carbohydrate is present at a concentration of about 25 mM. In some embodiments, the carbohydrate is present at a concentration of about 26 mM. In some embodiments, the carbohydrate is present at a concentration of about 27 mM. In some embodiments, the carbohydrate is present at a concentration of about 28 mM. In some embodiments, the carbohydrate is present at a concentration of about 29 mM. In some embodiments, the carbohydrate is present at a concentration of about 30 mM. In some embodiments, the carbohydrate is present at a concentration of about 31 mM. In some embodiments, the carbohydrate is present at a concentration of about 32 mM. In some embodiments, the carbohydrate is present at a concentration of about 33 mM. In some embodiments, the carbohydrate is present at a concentration of about 34 mM. In some embodiments, the carbohydrate is present at a concentration of about 35 mM. In some embodiments, the carbohydrate is present at a concentration of about 36 mM. In some embodiments, the carbohydrate is present at a concentration of about 37 mM. In some embodiments, the carbohydrate is present at a concentration of about 38 mM. In some embodiments, the carbohydrate is present at a concentration of about 39 mM. In some embodiments, the carbohydrate is present at a concentration of about 40 mM. In some embodiments, the carbohydrate is present at a concentration of about 41 mM. In some embodiments, the carbohydrate is present at a concentration of about 42 mM. In some embodiments, the carbohydrate is present at a concentration of about 43 mM. In some embodiments, the carbohydrate is present at a concentration of about 44 mM. In some embodiments, the carbohydrate is present at a concentration of about 45 mM.

In various embodiments, the carbohydrate is present at a concentration of about 2.0% to about 3.0% by weight per volume of the composition. In some embodiments, the carbohydrate is present at a concentration of about 2.1% to about 2.9% by weight per volume of the composition. In some embodiments, the carbohydrate is present at a concentration of about 2.2% to about 2.8% by weight per volume of the composition. In some embodiments, the carbohydrate is present at a concentration of about 2.3% to about 2.7% by weight per volume of the composition. In some embodiments, the carbohydrate is present at a concentration of about 2.4% to about 2.6% by weight per volume of the composition. In some embodiments, the carbohydrate is present at a concentration of about 2.5% by weight per volume of the composition. In certain embodiments, the carbohydrate is present at a concentration of 2.5% by weight per volume of the composition.

In various embodiments, the carbohydrate is present at a concentration of about 0.5% to about 1.5% by weight per volume of the composition. In some embodiments, the carbohydrate is present at a concentration of about 0.6% to about 1.4% by weight per volume of the composition. In some embodiments, the carbohydrate is present at a concentration of about 0.7% to about 1.3% by weight per volume of the composition. In some embodiments, the carbohydrate is present at a concentration of about 0.8% to about 1.2% by weight per volume of the composition. In some embodiments, the carbohydrate is present at a concentration of about 0.9% to about 1.1% by weight per volume of the composition.

In some embodiments, the carbohydrate is present at a concentration of about 0.5% by weight per volume of the composition. In some embodiments, the carbohydrate is present at a concentration of about 0.6% by weight per volume of the composition. In some embodiments, the carbohydrate is present at a concentration of about 0.7% by weight per volume of the composition. In some embodiments, the carbohydrate is present at a concentration of about 0.8% by weight per volume of the composition. In some embodiments, the carbohydrate is present at a concentration of about 0.9% by weight per volume of the composition. In some embodiments, the carbohydrate is present at a concentration of about 1.0%. In some embodiments, the carbohydrate is present at a concentration of about 1.1% by weight per volume of the composition. In some embodiments, the carbohydrate is present at a concentration of about 1.2% by weight per volume of the composition. In some embodiments, the carbohydrate is present at a concentration of about 1.3% by weight per volume of the composition. In some embodiments, the carbohydrate is present at a concentration of about 1.4% by weight per volume of the composition. In some embodiments, the carbohydrate is present at a concentration of about 1.5% by weight per volume of the composition. In some embodiments, the carbohydrate is present at a concentration of about 1.6% by weight per volume of the composition. In some embodiments, the carbohydrate is present at a concentration of about 1.7% by weight per volume of the composition. In some embodiments, the carbohydrate is present at a concentration of about 1.8% by weight per volume of the composition. In some embodiments, the carbohydrate is present at a concentration of about 1.9% by weight per volume of the composition. In some embodiments, the carbohydrate is present at a concentration of about 2% by weight per volume of the composition. In some embodiments, the carbohydrate is present at a concentration of about 2.1% by weight per volume of the composition. In some embodiments, the carbohydrate is present at a concentration of about 2.2% by weight per volume of the composition. In some embodiments, the carbohydrate is present at a concentration of about 2.3% by weight per volume of the composition. In some embodiments, the carbohydrate is present at a concentration of about 2.4% by weight per volume of the composition. In some embodiments, the carbohydrate is present at a concentration of about 2.5% by weight per volume of the composition. In some embodiments, the carbohydrate is present at a concentration of about 2.6% by weight per volume of the composition. In some embodiments, the carbohydrate is present at a concentration of about 2.7% by weight per volume of the composition. In some embodiments, the carbohydrate is present at a concentration of about 2.8% by weight per volume of the composition. In some embodiments, the carbohydrate is present at a concentration of about 2.9% by weight per volume of the composition. In some embodiments, the carbohydrate is present at a concentration of about 3% by weight per volume of the composition.

In various embodiments, the carbohydrate is present at a concentration of about 100 mM to about 135 mM. In some embodiments, the carbohydrate is present at a concentration of about 105 mM to about 130 mM. In some embodiments, the carbohydrate is present at a concentration of about 110 mM to about 125 mM. In some embodiments, the carbohydrate is present at a concentration of about 111 mM to about 124 mM. In some embodiments, the carbohydrate is present at a concentration of about 111 mM to about 123 mM. In some embodiments, the carbohydrate is present at a concentration of about 112 mM to about 122 mM. In some embodiments, the carbohydrate is present at a concentration of about 113 mM to about 121 mM. In some embodiments, the carbohydrate is present at a concentration of about 114 mM to about 120 mM. In some embodiments, the carbohydrate is present at a concentration of about 115 mM to about 119 mM. In some embodiments, the carbohydrate is present at a concentration of about 116 mM to about 118 mM.

In some embodiments, the carbohydrate is present at a concentration of about 100 mM. In some embodiments, the carbohydrate is present at a concentration of about 101 mM. In some embodiments, the carbohydrate is present at a concentration of about 102 mM. In some embodiments, the carbohydrate is present at a concentration of about 103 mM. In some embodiments, the carbohydrate is present at a concentration of about 104 mM. In some embodiments, the carbohydrate is present at a concentration of about 105 mM. In some embodiments, the carbohydrate is present at a concentration of about 106 mM. In some embodiments, the carbohydrate is present at a concentration of about 107 mM. In some embodiments, the carbohydrate is present at a concentration of about 108 mM. In some embodiments, the carbohydrate is present at a concentration of about 109 mM. In some embodiments, the carbohydrate is present at a concentration of about 110 mM. In some embodiments, the carbohydrate is present at a concentration of about 111 mM. In some embodiments, the carbohydrate is present at a concentration of about 112 mM. In some embodiments, the carbohydrate is present at a concentration of about 113 mM. In some embodiments, the carbohydrate is present at a concentration of about 114 mM. In some embodiments, the carbohydrate is present at a concentration of about 115 mM. In some embodiments, the carbohydrate is present at a concentration of about 116 mM. In some embodiments, the carbohydrate is present at a concentration of about 117 mM. In some embodiments, the carbohydrate is present at a concentration of about 118 mM. In some embodiments, the carbohydrate is present at a concentration of about 119 mM. In some embodiments, the carbohydrate is present at a concentration of about 120 mM. In some embodiments, the carbohydrate is present at a concentration of about 121 mM. In some embodiments, the carbohydrate is present at a concentration of about 122 mM. In some embodiments, the carbohydrate is present at a concentration of about 123 mM. In some embodiments, the carbohydrate is present at a concentration of about 124 mM. In some embodiments, the carbohydrate is present at a concentration of about 125 mM. In some embodiments, the carbohydrate is present at a concentration of about 126 mM. In some embodiments, the carbohydrate is present at a concentration of about 127 mM. In some embodiments, the carbohydrate is present at a concentration of about 128 mM. In some embodiments, the carbohydrate is present at a concentration of about 129 mM. In some embodiments, the carbohydrate is present at a concentration of about 130 mM. In some embodiments, the carbohydrate is present at a concentration of about 131 mM. In some embodiments, the carbohydrate is present at a concentration of about 132 mM. In some embodiments, the carbohydrate is present at a concentration of about 133 mM. In some embodiments, the carbohydrate is present at a concentration of about 134 mM. In some embodiments, the carbohydrate is present at a concentration of about 135 mM. In some embodiments, the carbohydrate is present at a concentration of about 136 mM. In some embodiments, the carbohydrate is present at a concentration of about 137 mM. In some embodiments, the carbohydrate is present at a concentration of about 138 mM. In some embodiments, the carbohydrate is present at a concentration of about 139 mM. In some embodiments, the carbohydrate is present at a concentration of about 140 mM. In some embodiments, the carbohydrate is present at a concentration of about 141 mM. In some embodiments, the carbohydrate is present at a concentration of about 142 mM. In some embodiments, the carbohydrate is present at a concentration of about 143 mM. In some embodiments, the carbohydrate is present at a concentration of about 144 mM. In some embodiments, the carbohydrate is present at a concentration of about 145 mM. In some embodiments, the carbohydrate is present at a concentration of about 146 mM. In some embodiments, the carbohydrate is present at a concentration of about 147 mM. In some embodiments, the carbohydrate is present at a concentration of about 148 mM. In some embodiments, the carbohydrate is present at a concentration of about 149 mM. In some embodiments, the carbohydrate is present at a concentration of about 150 mM.

In various embodiments, the carbohydrate is present at a concentration of about 265 mM to about 285 mM. In some embodiments, the carbohydrate is present at a concentration of about 266 mM to about 284 mM. In some embodiments, the carbohydrate is present at a concentration of about 267 mM to about 283 mM. In some embodiments, the carbohydrate is present at a concentration of about 268 mM to about 282 mM. In some embodiments, the carbohydrate is present at a concentration of about 269 mM to about 281 mM. In some embodiments, the carbohydrate is present at a concentration of about 270 mM to about 280 mM. In some embodiments, the carbohydrate is present at a concentration of about 271 mM to about 279 mM. In some embodiments, the carbohydrate is present at a concentration of about 272 mM to about 278 mM. In some embodiments, the carbohydrate is present at a concentration of about 273 mM to about 277 mM. In some embodiments, the carbohydrate is present at a concentration of about 274 mM to about 276 mM.

In some embodiments, the carbohydrate is present at a concentration of about 265 mM. In some embodiments, the carbohydrate is present at a concentration of about 266 mM. In some embodiments, the carbohydrate is present at a concentration of about 267 mM. In some embodiments, the carbohydrate is present at a concentration of about 268 mM. In some embodiments, the carbohydrate is present at a concentration of about 269 mM. In some embodiments, the carbohydrate is present at a concentration of about 270 mM. In some embodiments, the carbohydrate is present at a concentration of about 271 mM. In some embodiments, the carbohydrate is present at a concentration of about 272 mM. In some embodiments, the carbohydrate is present at a concentration of about 273 mM. In some embodiments, the carbohydrate is present at a concentration of about 274 mM. In some embodiments, the carbohydrate is present at a concentration of about 275 mM. In some embodiments, the carbohydrate is present at a concentration of about 276 mM. In some embodiments, the carbohydrate is present at a concentration of about 277 mM. In some embodiments, the carbohydrate is present at a concentration of about 278 mM. In some embodiments, the carbohydrate is present at a concentration of about 279 mM. In some embodiments, the carbohydrate is present at a concentration of about 280 mM. In some embodiments, the carbohydrate is present at a concentration of about 281 mM. In some embodiments, the carbohydrate is present at a concentration of about 282 mM. In some embodiments, the carbohydrate is present at a concentration of about 283 mM. In some embodiments, the carbohydrate is present at a concentration of about 284 mM. In some embodiments, the carbohydrate is present at a concentration of about 285 mM.

In various embodiments, the carbohydrate is present at a concentration of about 3.5% to about 4.5% by weight per volume of the composition. In some embodiments, the carbohydrate is present at a concentration of about 3.6% to about 4.4% by weight per volume of the composition. In some embodiments, the carbohydrate is present at a concentration of about 3.7% to about 4.3% by weight per volume of the composition. In some embodiments, the carbohydrate is present at a concentration of about 3.8% to about 4.2% by weight per volume of the composition. In some embodiments, the carbohydrate is present at a concentration of about 3.9% to about 4.1% by weight per volume of the composition.

In some embodiments, the carbohydrate is present at a concentration of about 3.5% by weight per volume of the composition. In some embodiments, the carbohydrate is present at a concentration of about 3.6% by weight per volume of the composition. In some embodiments, the carbohydrate is present at a concentration of about 3.7% by weight per volume of the composition. In some embodiments, the carbohydrate is present at a concentration of about 3.8% by weight per volume of the composition. In some embodiments, the carbohydrate is present at a concentration of about 3.9% by weight per volume of the composition. In some embodiments, the carbohydrate is present at a concentration of about 4.0%. In some embodiments, the carbohydrate is present at a concentration of about 4.1% by weight per volume of the composition. In some embodiments, the carbohydrate is present at a concentration of about 4.2% by weight per volume of the composition. In some embodiments, the carbohydrate is present at a concentration of about 4.3% by weight per volume of the composition. In some embodiments, the carbohydrate is present at a concentration of about 4.4% by weight per volume of the composition. In some embodiments, the carbohydrate is present at a concentration of about 4.5% by weight per volume of the composition.

In particular embodiments, the carbohydrate is a disaccharide. In various embodiments, the carbohydrate is lactose, glucose, mannose, mannitol, sorbitol, sucrose, trehalose, inulin, and/or glycerol. In some embodiments, the carbohydrate is sucrose and/or trehalose. In particular embodiments, the carbohydrate is sucrose. In particular embodiments, the carbohydrate is trehalose.

In various embodiments, the composition comprises an amino acid. In some embodiments, the amino acid is present at a concentration of about 40 mM to about 60 mM. In some embodiments, the amino acid is present at a concentration of about 41 mM to about 59 mM. In some embodiments, the amino acid is present at a concentration of about 42 mM to about 58 mM. In some embodiments, the amino acid is present at a concentration of about 43 mM to about 57 mM. In some embodiments, the amino acid is present at a concentration of about 44 mM to about 56 mM. In some embodiments, the amino acid is present at a concentration of about 45 mM to about 55 mM. In some embodiments, the amino acid is present at a concentration of about 46 mM to about 54 mM. In some embodiments, the amino acid is present at a concentration of about 47 mM to about 53 mM. In some embodiments, the amino acid is present at a concentration of about 48 mM to about 52 mM. In some embodiments, the amino acid is present at a concentration of about 49 mM to about 51 mM.

In some embodiments, the amino acid is present at a concentration of about 40 mM. In some embodiments, the amino acid is present at a concentration of about 41 mM. In some embodiments, the amino acid is present at a concentration of about 42 mM. In some embodiments, the amino acid is present at a concentration of about 43 mM. In some embodiments, the amino acid is present at a concentration of about 44 mM. In some embodiments, the amino acid is present at a concentration of about 45 mM. In some embodiments, the amino acid is present at a concentration of about 46 mM. In some embodiments, the amino acid is present at a concentration of about 47 mM. In some embodiments, the amino acid is present at a concentration of about 48 mM. In some embodiments, the amino acid is present at a concentration of about 49 mM. In some embodiments, the amino acid is present at a concentration of about 50 mM. In certain embodiments, the amino acid is present at a concentration of 50 mM. In some embodiments, the amino acid is present at a concentration of about 51 mM. In some embodiments, the amino acid is present at a concentration of about 52 mM. In some embodiments, the amino acid is present at a concentration of about 53 mM. In some embodiments, the amino acid is present at a concentration of about 54 mM. In some embodiments, the amino acid is present at a concentration of about 55 mM. In some embodiments, the amino acid is present at a concentration of about 56 mM. In some embodiments, the amino acid is present at a concentration of about 57 mM. In some embodiments, the amino acid is present at a concentration of about 58 mM. In some embodiments, the amino acid is present at a concentration of about 59 mM. In some embodiments, the amino acid is present at a concentration of about 60 mM.

In various embodiments, the amino acid is present at a concentration of about 15 mM to about 35 mM. In some embodiments, the amino acid is present at a concentration of about 16 mM to about 34 mM. In some embodiments, the amino acid is present at a concentration of about 17 mM to about 33 mM. In some embodiments, the amino acid is present at a concentration of about 18 mM to about 32 mM. In some embodiments, the amino acid is present at a concentration of about 19 mM to about 31 mM. In some embodiments, the amino acid is present at a concentration of about 20 mM to about 30 mM. In some embodiments, the amino acid is present at a concentration of about 21 mM to about 29 mM. In some embodiments, the amino acid is present at a concentration of about 22 mM to about 28 mM. In some embodiments, the amino acid is present at a concentration of about 23 mM to about 27 mM. In some embodiments, the amino acid is present at a concentration of about 24 mM to about 26 mM.

In some embodiments, the amino acid is present at a concentration of about 15 mM. In some embodiments, the amino acid is present at a concentration of about 16 mM. In some embodiments, the amino acid is present at a concentration of about 17 mM. In some embodiments, the amino acid is present at a concentration of about 18 mM. In some embodiments, the amino acid is present at a concentration of about 19 mM. In some embodiments, the amino acid is present at a concentration of about 20 mM. In some embodiments, the amino acid is present at a concentration of about 21 mM. In some embodiments, the amino acid is present at a concentration of about 22 mM. In some embodiments, the amino acid is present at a concentration of about 23 mM. In some embodiments, the amino acid is present at a concentration of about 24 mM. In some embodiments, the amino acid is present at a concentration of about 25 mM. In some embodiments, the amino acid is present at a concentration of about 26 mM. In some embodiments, the amino acid is present at a concentration of about 27 mM. In some embodiments, the amino acid is present at a concentration of about 28 mM. In some embodiments, the amino acid is present at a concentration of about 29 mM. In some embodiments, the amino acid is present at a concentration of about 30 mM. In some embodiments, the amino acid is present at a concentration of about 31 mM. In some embodiments, the amino acid is present at a concentration of about 32 mM. In some embodiments, the amino acid is present at a concentration of about 33 mM. In some embodiments, the amino acid is present at a concentration of about 34 mM. In some embodiments, the amino acid is present at a concentration of about 35 mM.

In various embodiments, the amino acid is present at a concentration of about 65 mM to about 85 mM. In some embodiments, the amino acid is present at a concentration of about 66 mM to about 84 mM. In some embodiments, the amino acid is present at a concentration of about 67 mM to about 83 mM. In some embodiments, the amino acid is present at a concentration of about 68 mM to about 82 mM. In some embodiments, the amino acid is present at a concentration of about 69 mM to about 81 mM. In some embodiments, the amino acid is present at a concentration of about 70 mM to about 80 mM. In some embodiments, the amino acid is present at a concentration of about 71 mM to about 79 mM. In some embodiments, the amino acid is present at a concentration of about 72 mM to about 78 mM. In some embodiments, the amino acid is present at a concentration of about 73 mM to about 77 mM. In some embodiments, the amino acid is present at a concentration of about 74 mM to about 76 mM.

In some embodiments, the amino acid is present at a concentration of about 65 mM. In some embodiments, the amino acid is present at a concentration of about 66 mM. In some embodiments, the amino acid is present at a concentration of about 67 mM. In some embodiments, the amino acid is present at a concentration of about 68 mM. In some embodiments, the amino acid is present at a concentration of about 69 mM. In some embodiments, the amino acid is present at a concentration of about 70 mM. In some embodiments, the amino acid is present at a concentration of about 71 mM. In some embodiments, the amino acid is present at a concentration of about 72 mM. In some embodiments, the amino acid is present at a concentration of about 73 mM. In some embodiments, the amino acid is present at a concentration of about 74 mM. In some embodiments, the amino acid is present at a concentration of about 75 mM. In some embodiments, the amino acid is present at a concentration of about 76 mM. In some embodiments, the amino acid is present at a concentration of about 77 mM. In some embodiments, the amino acid is present at a concentration of about 78 mM. In some embodiments, the amino acid is present at a concentration of about 79 mM. In some embodiments, the amino acid is present at a concentration of about 80 mM. In some embodiments, the amino acid is present at a concentration of about 81 mM. In some embodiments, the amino acid is present at a concentration of about 82 mM. In some embodiments, the amino acid is present at a concentration of about 83 mM. In some embodiments, the amino acid is present at a concentration of about 84 mM. In some embodiments, the amino acid is present at a concentration of about 85 mM.

In some embodiments, the amino acid is a non-polar amino acid. In some embodiments, the amino acid is selected from the group consisting of: glycine, alanine, valine, leucine, methionine, isoleucine phenylalanine, tyrosine, and tryptophan. In some embodiments, the amino acid is selected from the group consisting of: phenylalanine, tyrosine, tryptophan, and proline. In some embodiments, the amino acid is phenylalanine. In some embodiments, the amino acid is tyrosine. In some embodiments, the amino acid is tryptophan. In some embodiments, the amino acid is proline. In particular embodiments, the amino acid is L-Proline.

In various embodiments, the composition further comprises a salt. In some embodiments, the salt is present at a concentration of about 65 mM to about 85 mM. In some embodiments, the salt is present at a concentration of about 66 mM to about 84 mM. In some embodiments, the salt is present at a concentration of about 67 mM to about 83 mM. In some embodiments, the salt is present at a concentration of about 68 mM to about 82 mM. In some embodiments, the salt is present at a concentration of about 69 mM to about 81 mM. In some embodiments, the salt is present at a concentration of about 70 mM to about 80 mM. In some embodiments, the salt is present at a concentration of about 71 mM to about 79 mM. In some embodiments, the salt is present at a concentration of about 72 mM to about 78 mM. In some embodiments, the salt is present at a concentration of about 73 mM to about 77 mM. In some embodiments, the salt is present at a concentration of about 74 mM to about 76 mM.

In some embodiments, the salt is present at a concentration of about 65 mM. In some embodiments, the salt is present at a concentration of about 66 mM. In some embodiments, the salt is present at a concentration of about 67 mM. In some embodiments, the salt is present at a concentration of about 68 mM. In some embodiments, the salt is present at a concentration of about 69 mM. In some embodiments, the salt is present at a concentration of about 70 mM. In some embodiments, the salt is present at a concentration of about 71 mM. In some embodiments, the salt is present at a concentration of about 72 mM. In some embodiments, the salt is present at a concentration of about 73 mM. In some embodiments, the salt is present at a concentration of about 74 mM. In some embodiments, the salt is present at a concentration of about 75 mM. In certain embodiments, the salt is present at a concentration of 75 mM. In some embodiments, the salt is present at a concentration of about 76 mM. In some embodiments, the salt is present at a concentration of about 77 mM. In some embodiments, the salt is present at a concentration of about 78 mM. In some embodiments, the salt is present at a concentration of about 79 mM. In some embodiments, the salt is present at a concentration of about 80 mM. In some embodiments, the salt is present at a concentration of about 81 mM. In some embodiments, the salt is present at a concentration of about 82 mM. In some embodiments, the salt is present at a concentration of about 83 mM. In some embodiments, the salt is present at a concentration of about 84 mM. In some embodiments, the salt is present at a concentration of about 85 mM. In certain embodiments, the salt is a chlorine salt. In particular embodiments, the salt is KCl. In particular embodiments, the salt is NaCl.

In various embodiments, the composition further comprises a poloxamer. “Poloxamer” refers to a non-ionic triblock copolymer composed of a central hydrophobic chain of polyoxypropylene flanked by two hydrophilic chains of polyoxyethylene. Poloxamers are also known by the trade name of “Pluronics” or “Synperonics” (BASF). The block copolymer can be represented by the following formula: HO(C₂H₄O)ₓ(C₃H₆O)_{y}(C₂H₄O)_{z}H.

The synthesis of block copolymers yields a population of polymers that have an average molecular weight. Thus, in particular embodiments, the term “poloxamer” as used herein can be used interchangeably with the term “poloxamers” (representing an entity of several poloxamers, also referred to as mixture of poloxamers) if not explicitly stated otherwise. The term “average” in relation to the number of monomer units or molecular weight of (a) poloxamer(s) as used herein is a consequence of the technical inability to produce poloxamers all having the identical composition and thus the identical molecular weight. Poloxamers produced according to state-of-the-art methods will be present as a mixture of poloxamers each showing a variability as regards their molecular weight, but the mixture as a whole averaging the molecular weight specified herein. BASF and Sigma Aldrich are suitable sources of poloxamers for use in particular embodiments contemplated herein.

In some embodiments, the poloxamer is present at a concentration of about 0.01 mg/ml to about 2 mg/ml. In some embodiments, the poloxamer is present at a concentration of about 0.1 mg/ml to about 2 mg/ml. In some embodiments, the poloxamer is present at a concentration of about 0.2 mg/ml to about 2 mg/ml. In some embodiments, the poloxamer is present at a concentration of about 0.3 mg/ml to about 2 mg/ml.

In some embodiments, the poloxamer is present at a concentration of about 0.01 mg/ml to about 1 mg/ml. In some embodiments, the poloxamer is present at a concentration of about 0.1 mg/ml to about 1 mg/ml. In some embodiments, the poloxamer is present at a concentration of about 0.1 mg/ml to about 0.8 mg/ml. In some embodiments, the poloxamer is present at a concentration of about 0.2 mg/ml to about 1 mg/ml. In some embodiments, the poloxamer is present at a concentration of about 0.3 mg/ml to about 1 mg/ml. In some embodiments, the poloxamer is present at a concentration of about 0.02 mg/ml to about 0.9 mg/ml. In some embodiments, the poloxamer is present at a concentration of about 0.03 mg/ml to about 0.8 mg/ml. In some embodiments, the poloxamer is present at a concentration of about 0.04 mg/ml to about 0.7 mg/ml. In some embodiments, the poloxamer is present at a concentration of about 0.05 mg/ml to about 0.6 mg/ml. In some embodiments, the poloxamer is present at a concentration of about 0.06 mg/ml to about 0.5 mg/ml. In some embodiments, the poloxamer is present at a concentration of about 0.07 mg/ml to about 0.4 mg/ml. In some embodiments, the poloxamer is present at a concentration of about 0.08 mg/ml to about 0.3 mg/ml. In some embodiments, the poloxamer is present at a concentration of about 0.09 mg/ml to about 0.2 mg/ml. In some embodiments, the poloxamer is present at a concentration of about 0.1 mg/ml to about 0.5 mg/ml. In some embodiments, the poloxamer is present at a concentration of about 0.2 mg/ml to about 0.4 mg/ml.

In certain embodiments, the poloxamer is present at a concentration of about 0.1 mg/ml. In certain embodiments, the poloxamer is present at a concentration of about 0.2 mg/ml. In certain embodiments, the poloxamer is present at a concentration of about 0.3 mg/ml. In certain embodiments, the poloxamer is present at a concentration of about 0.4 mg/ml. In certain embodiments, the poloxamer is present at a concentration of about 0.5 mg/ml. In certain embodiments, the poloxamer is present at a concentration of about 0.6 mg/ml. In certain embodiments, the poloxamer is present at a concentration of about 0.7 mg/ml. In certain embodiments, the poloxamer is present at a concentration of about 0.8 mg/ml. In certain embodiments, the poloxamer is present at a concentration of about 0.9 mg/ml. In certain embodiments, the poloxamer is present at a concentration of about 1.0 mg/ml. In certain embodiments, the poloxamer is present at a concentration of about 1.1 mg/ml. In certain embodiments, the poloxamer is present at a concentration of about 1.2 mg/ml. In certain embodiments, the poloxamer is present at a concentration of about 1.3 mg/ml. In certain embodiments, the poloxamer is present at a concentration of about 1.4 mg/ml. In certain embodiments, the poloxamer is present at a concentration of about 1.5 mg/ml. In certain embodiments, the poloxamer is present at a concentration of about 1.6 mg/ml. In certain embodiments, the poloxamer is present at a concentration of about 1.7 mg/ml. In certain embodiments, the poloxamer is present at a concentration of about 1.8 mg/ml. In certain embodiments, the poloxamer is present at a concentration of about 1.9 mg/ml. In certain embodiments, the poloxamer is present at a concentration of about 2.0 mg/ml. In some embodiments, the poloxamer is present at a concentration of about 0.1 mg/ml. In certain embodiments, the poloxamer is present at a concentration of 0.1 mg/ml. In some embodiments, the poloxamer is poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, or poloxamer 407. In particular embodiments, the poloxamer is poloxamer 188 (P188). In certain embodiments, the poloxamer is poloxamer 407.

In various embodiments, the composition comprises a pH of about 6.5 to about 8.0. In some embodiments, the composition comprises a pH of about 6.5. In some embodiments, the composition comprises a pH of about 6.6. In some embodiments, the composition comprises a pH of about 6.7. In some embodiments, the composition comprises a pH of about 6.8. In some embodiments, the composition comprises a pH of about 6.9. In some embodiments, the composition comprises a pH of about 7.0. In some embodiments, the composition comprises a pH of about 7.1. In some embodiments, the composition comprises a pH of about 7.2. In some embodiments, the composition comprises a pH of about 7.3. In some embodiments, the composition comprises a pH of about 7.4. In some embodiments, the composition comprises a pH of about 7.5. In some embodiments, the composition comprises a pH of about 7.6. In some embodiments, the composition comprises a pH of about 7.7. In some embodiments, the composition comprises a pH of about 7.8. In some embodiments, the composition comprises a pH of about 7.9. In some embodiments, the composition comprises a pH of about 8.0.

In a particular embodiment, an aqueous viral composition is provided, wherein the composition comprises a viral vector; about 27.5 mM HEPES; about 73 mM sucrose or about 2.5% sucrose by weight per volume of composition; and about 50 mM L-Proline; wherein the composition comprises a pH of about 7.

In a particular embodiment, an aqueous viral composition is provided, wherein the composition comprises a viral vector; about 27.5 mM L-Histidine; about 73 mM sucrose or about 2.5% sucrose by weight per volume of composition; and about 50 mM L-Proline;

wherein the composition comprises a pH of about 7.

In a particular embodiment, an aqueous viral composition is provided, wherein the composition comprises a viral vector; about 27.5 mM HEPES; about 73 mM sucrose or about 2.5% sucrose by weight per volume of composition; about 50 mM L-Proline; and about 0.1 to about 0.8 mg/mL poloxamer 188; wherein the composition comprises a pH of about 7.

In a particular embodiment, an aqueous viral composition is provided, wherein the composition comprises a viral vector; about 27.5 mM HEPES; about 73 mM sucrose or about 2.5% sucrose by weight per volume of composition; about 50 mM L-Proline; and about 75 mM NaCl; wherein the composition comprises a pH of about 7.

In a particular embodiment, an aqueous viral composition is provided, wherein the composition comprises a viral vector; about 27.5 mM HEPES; about 73 mM sucrose or about 2.5% sucrose by weight per volume of composition; about 50 mM L-Proline; about 75 mM NaCl; and about 0.1 to about 0.8 mg/mL poloxamer 188; wherein the composition comprises a pH of about 7.

In a particular embodiment, an aqueous viral composition is provided, wherein the composition comprises a viral vector; about 27.5 mM HEPES; about 73 mM sucrose or about 2.5% sucrose by weight per volume of composition; about 75 mM NaCl; and about 0.1 to about 0.8 mg/ml poloxamer 188 or about 0.01% poloxamer 188 by weight per volume of composition; wherein the composition comprises a pH of about 7.

In a particular embodiment, an aqueous viral composition is provided, wherein the composition comprises a viral vector; about 27.5 mM HEPES; about 73 mM sucrose or about 2.5% sucrose by weight per volume of composition; and about 75 mM NaCl; wherein the composition comprises a pH of about 7.

In any of the embodiments contemplated herein, the viral vector is present at a titer or concentration from about 1×10⁸ to about 2×10⁹ TU/ml. In some embodiments, the viral vector is present at a titer or concentration of about 1×10⁸ TU/ml. In some embodiments, the viral vector is present at a titer or concentration of about 2×10⁸ TU/ml. In some embodiments, the viral vector is present at a titer or concentration of about 3×10⁸ TU/ml. In some embodiments, the viral vector is present at a titer or concentration of about 4×10⁸ TU/ml. In some embodiments, the viral vector is present at a titer or concentration of about 5×10⁸ TU/ml. In some embodiments, the viral vector is present at a titer or concentration of about 6×10⁸ TU/ml. In some embodiments, the viral vector is present at a titer or concentration of about 7×10⁸ TU/ml. In some embodiments, the viral vector is present at a titer or concentration of about 8×10⁸ TU/ml. In some embodiments, the viral vector is present at a titer or concentration of about 9×10⁸ TU/ml. In some embodiments, the viral vector is present at a titer or concentration of about 1×10⁹ TU/ml. In some embodiments, the viral vector is present at a titer or concentration of about 2×10⁹ TU/ml.

In certain embodiments contemplated herein, the formulations do not comprise particular components. Accordingly, in any of the embodiments contemplated herein, the composition does not comprise PIPES. In any of the embodiments contemplated herein, the composition does not comprise sodium citrate. In any of the embodiments contemplated herein, the composition does not comprise sodium phosphate. In any of the embodiments contemplated herein, the composition does not comprise Tris. In any of the embodiments contemplated herein, the composition does not comprise a salt. In any of the embodiments contemplated herein, the composition does not comprise NaCl. In any of the embodiments contemplated herein, the composition does not comprise KCl. In any of the embodiments contemplated herein, the composition does not comprise serum. In any of the embodiments contemplated herein, the composition does not comprise human serum albumin (HSA). In any of the embodiments contemplated herein, the compositions contemplated herein do not comprise trehalose.

In any of the embodiments contemplated herein, the viral vector maintains greater than about 75% infectious titer recover in HOS cells after storage and/or a freeze-thaw cycle relative to the infectious titer of the viral vector in the composition prior to storage or freeze-thaw. In any of the embodiments contemplated herein, the viral vector maintains greater than about 76% infectious titer recover in HOS cells after storage and/or a freeze-thaw cycle relative to the infectious titer of the viral vector in the composition prior to storage or freeze-thaw. In any of the embodiments contemplated herein, the viral vector maintains greater than about 77% infectious titer recover in HOS cells after storage and/or a freeze-thaw cycle relative to the infectious titer of the viral vector in the composition prior to storage or freeze-thaw. In any of the embodiments contemplated herein, the viral vector maintains greater than about 78% infectious titer recover in HOS cells after storage and/or a freeze-thaw cycle relative to the infectious titer of the viral vector in the composition prior to storage or freeze-thaw. In any of the embodiments contemplated herein, the viral vector maintains greater than about 79% infectious titer recover in HOS cells after storage and/or a freeze-thaw cycle relative to the infectious titer of the viral vector in the composition prior to storage or freeze-thaw. In any of the embodiments contemplated herein, the viral vector maintains greater than about 80% infectious titer recover in HOS cells after storage and/or a freeze-thaw cycle relative to the infectious titer of the viral vector in the composition prior to storage or freeze-thaw. In any of the embodiments contemplated herein, the viral vector maintains greater than about 81% infectious titer recover in HOS cells after storage and/or a freeze-thaw cycle relative to the infectious titer of the viral vector in the composition prior to storage or freeze-thaw. In any of the embodiments contemplated herein, the viral vector maintains greater than about 82% infectious titer recover in HOS cells after storage and/or a freeze-thaw cycle relative to the infectious titer of the viral vector in the composition prior to storage or freeze-thaw. In any of the embodiments contemplated herein, the viral vector maintains greater than about 83% infectious titer recover in HOS cells after storage and/or a freeze-thaw cycle relative to the infectious titer of the viral vector in the composition prior to storage or freeze-thaw. In any of the embodiments contemplated herein, the viral vector maintains greater than about 84% infectious titer recover in HOS cells after storage and/or a freeze-thaw cycle relative to the infectious titer of the viral vector in the composition prior to storage or freeze-thaw. In any of the embodiments contemplated herein, the viral vector maintains greater than about 85% infectious titer recover in HOS cells after storage and/or a freeze-thaw cycle relative to the infectious titer of the viral vector in the composition prior to storage or freeze-thaw. In any of the embodiments contemplated herein, the viral vector maintains greater than about 86% infectious titer recover in HOS cells after storage and/or a freeze-thaw cycle relative to the infectious titer of the viral vector in the composition prior to storage or freeze-thaw. In any of the embodiments contemplated herein, the viral vector maintains greater than about 87% infectious titer recover in HOS cells after storage and/or a freeze-thaw cycle relative to the infectious titer of the viral vector in the composition prior to storage or freeze-thaw. In any of the embodiments contemplated herein, the viral vector maintains greater than about 88% infectious titer recover in HOS cells after storage and/or a freeze-thaw cycle relative to the infectious titer of the viral vector in the composition prior to storage or freeze-thaw. In any of the embodiments contemplated herein, the viral vector maintains greater than about 89% infectious titer recover in HOS cells after storage and/or a freeze-thaw cycle relative to the infectious titer of the viral vector in the composition prior to storage or freeze-thaw. In any of the embodiments contemplated herein, the viral vector maintains greater than about 90% infectious titer recover in HOS cells after storage and/or a freeze-thaw cycle relative to the infectious titer of the viral vector in the composition prior to storage or freeze-thaw. In any of the embodiments contemplated herein, the viral vector maintains greater than about 91% infectious titer recover in HOS cells after storage and/or a freeze-thaw cycle relative to the infectious titer of the viral vector in the composition prior to storage or freeze-thaw. In any of the embodiments contemplated herein, the viral vector maintains greater than about 92% infectious titer recover in HOS cells after storage and/or a freeze-thaw cycle relative to the infectious titer of the viral vector in the composition prior to storage or freeze-thaw. In any of the embodiments contemplated herein, the viral vector maintains greater than about 93% infectious titer recover in HOS cells after storage and/or a freeze-thaw cycle relative to the infectious titer of the viral vector in the composition prior to storage or freeze-thaw. In any of the embodiments contemplated herein, the viral vector maintains greater than about 94% infectious titer recover in HOS cells after storage and/or a freeze-thaw cycle relative to the infectious titer of the viral vector in the composition prior to storage or freeze-thaw. In any of the embodiments contemplated herein, the viral vector maintains greater than about 95% infectious titer recover in HOS cells after storage and/or a freeze-thaw cycle relative to the infectious titer of the viral vector in the composition prior to storage or freeze-thaw.

In any of the embodiments contemplated herein, the viral vectors have a thermal unfolding temperature of about 56° to about 62° C. as measured by differential scanning fluorimetry (DSF). In any of the embodiments contemplated herein, the viral vectors have a thermal unfolding temperature of about 58° to about 60° C. as measured by differential scanning fluorimetry (DSF). In any of the embodiments contemplated herein, the viral vectors have a thermal unfolding temperature of about 56° as measured by differential scanning fluorimetry (DSF) after. In any of the embodiments contemplated herein, the viral vectors have a thermal unfolding temperature of about 57° as measured by differential scanning fluorimetry (DSF) after. In any of the embodiments contemplated herein, the viral vectors have a thermal unfolding temperature of about 58° as measured by differential scanning fluorimetry (DSF). In any of the embodiments contemplated herein, the viral vectors have a thermal unfolding temperature of about 59° as measured by differential scanning fluorimetry (DSF). In any of the embodiments contemplated herein, the viral vectors have a thermal unfolding temperature of about 60° as measured by differential scanning fluorimetry (DSF). In any of the embodiments contemplated herein, the viral vectors have a thermal unfolding temperature of about 61° as measured by differential scanning fluorimetry (DSF). In any of the embodiments contemplated herein, the viral vectors have a thermal unfolding temperature of about 62° as measured by differential scanning fluorimetry (DSF).

In any of the embodiments contemplated herein, the viral vectors maintain a hydrodynamic diameter of about 150 nm to about 170 nm as measured by dynamic light scattering (DLS) at 25° C. and a viscosity value of 0.967 centipoise (cP), after storage and/or at least one freeze-thaw cycle; relative to hydrodynamic diameter of the viral vector in the composition prior to storage or at least one freeze-thaw cycle.

In any of the embodiments contemplated herein, the viral vectors maintain at least 78% potency as measured by transgene expression in PBMCs compared to a reference standard. In any of the embodiments contemplated herein, the viral vectors maintain at least 79% potency as measured by transgene expression in PBMCs compared to a reference standard. In any of the embodiments contemplated herein, the viral vectors maintain at least 80% potency as measured by transgene expression in PBMCs compared to a reference standard. In any of the embodiments contemplated herein, the viral vectors maintain at least 81% potency as measured by transgene expression in PBMCs compared to a reference standard. In any of the embodiments contemplated herein, the viral vectors maintain at least 82% potency as measured by transgene expression in PBMCs compared to a reference standard. In any of the embodiments contemplated herein, the viral vectors maintain at least 83% potency as measured by transgene expression in PBMCs compared to a reference standard. In any of the embodiments contemplated herein, the viral vectors maintain at least 84% potency as measured by transgene expression in PBMCs compared to a reference standard. In any of the embodiments contemplated herein, the viral vectors maintain at least 85% potency as measured by transgene expression in PBMCs compared to a reference standard. In any of the embodiments contemplated herein, the viral vectors maintain at least 86% potency as measured by transgene expression in PBMCs compared to a reference standard. In any of the embodiments contemplated herein, the viral vectors maintain at least 87% potency as measured by transgene expression in PBMCs compared to a reference standard. In any of the embodiments contemplated herein, the viral vectors maintain at least 88% potency as measured by transgene expression in PBMCs compared to a reference standard. In any of the embodiments contemplated herein, the viral vectors maintain at least 89% potency as measured by transgene expression in PBMCs compared to a reference standard. In any of the embodiments contemplated herein, the viral vectors maintain at least 90% potency as measured by transgene expression in PBMCs compared to a reference standard. In any of the embodiments contemplated herein, the viral vectors maintain at least 91% potency as measured by transgene expression in PBMCs compared to a reference standard. In any of the embodiments contemplated herein, the viral vectors maintain at least 92% potency as measured by transgene expression in PBMCs compared to a reference standard. In any of the embodiments contemplated herein, the viral vectors maintain at least 93% potency as measured by transgene expression in PBMCs compared to a reference standard. In any of the embodiments contemplated herein, the viral vectors maintain at least 94% potency as measured by transgene expression in PBMCs compared to a reference standard. In any of the embodiments contemplated herein, the viral vectors maintain at least 95% potency as measured by transgene expression in PBMCs compared to a reference standard.

In any of the aspects and embodiments contemplated herein, the composition has no visible particles or visible fiber particles (wispy fibers) after storage. In any of the aspects and embodiments contemplated herein, the composition has 5 or fewer visible particles or specs after storage. In any of the aspects and embodiments contemplated herein, the composition has 4 or fewer visible particles or specs after storage. In any of the aspects and embodiments contemplated herein, the composition has 3 or fewer visible particles or specs after storage. In any of the aspects and embodiments contemplated herein, the composition has 2 or fewer visible particles or specs after storage. In any of the aspects and embodiments contemplated herein, the composition has 1 or fewer visible particles or specs after storage. In any of the aspects and embodiments contemplated herein, the composition has no visible particles after storage.

In any of the aspects and embodiments contemplated herein, the composition has no visible particles or visible fiber particles (wispy fibers) after storage per 50 ml of composition. In any of the aspects and embodiments contemplated herein, the composition has 5 or fewer visible particles or specs after storage per 50 ml of composition. In any of the aspects and embodiments contemplated herein, the composition has 4 or fewer visible particles or specs after storage per 50 ml of composition. In any of the aspects and embodiments contemplated herein, the composition has 3 or fewer visible particles or specs after storage per 50 ml of composition. In any of the aspects and embodiments contemplated herein, the composition has 2 or fewer visible particles or specs after storage per 50 ml of composition. In any of the aspects and embodiments contemplated herein, the composition has 1 or fewer visible particles or specs after storage per 50 ml of composition. In any of the aspects and embodiments contemplated herein, the composition has no visible particles after storage per 50 ml of composition.

In any of the aspects and embodiments contemplated herein, the composition has no visible particles or visible fiber particles (wispy fibers) after storage per 36 ml of composition. In any of the aspects and embodiments contemplated herein, the composition has 5 or fewer visible particles or specs after storage per 36 ml of composition. In any of the aspects and embodiments contemplated herein, the composition has 4 or fewer visible particles or specs after storage per 36 ml of composition. In any of the aspects and embodiments contemplated herein, the composition has 3 or fewer visible particles or specs after storage per 36 ml of composition. In any of the aspects and embodiments contemplated herein, the composition has 2 or fewer visible particles or specs after storage per 36 ml of composition. In any of the aspects and embodiments contemplated herein, the composition has 1 or fewer visible particles or specs after storage per 36 ml of composition. In any of the aspects and embodiments contemplated herein, the composition has no visible particles after storage per 36 ml of composition.

In various embodiments, the storage is at 2-8° C. In some embodiments, the storage is at 2° C. In some embodiments, the storage is at 3° C. In some embodiments, the storage is at 4° C. In some embodiments, the storage is at 5° C. In some embodiments, the storage is at 6° C. In some embodiments, the storage is at 7° C. In some embodiments, the storage is at 8° C. In various embodiments, the storage is at 25° C. In various embodiments, the storage is at 37° C.

In various embodiments, the storage is for 24 hours, 48 hours, or 72, 96, 120, 144, or 168 hours, or more. In some embodiments, the storage is for at least 24 hours. In some embodiments, the storage is for at least 48 hours. In some embodiments, the storage is for at least 72 hours. In some embodiments, the storage is for at least 96 hours. In some embodiments, the storage is for at least 120 hours. In some embodiments, the storage is for at least 144 hours. In some embodiments, the storage is for at least 168 hours, or 1 week).

In some embodiments, the storage is for at least 1 week. In some embodiments, the storage is for at least 2 weeks. In some embodiments, the storage is for at least 3 weeks. In some embodiments, the storage is for at least 4 weeks. In some embodiments, the storage is for at least 1 month. In some embodiments, the storage is for at least 2 months. In some embodiments, the storage is for at least 3 months. In some embodiments, the storage is for at least 4 months. In some embodiments, the storage is for at least 5 months. In some embodiments, the storage is for at least 6 months. In some embodiments, the storage is for at least 7 months. In some embodiments, the storage is for at least 8 months. In some embodiments, the storage is for at least 9 months. In some embodiments, the storage is for at least 10 months. In some embodiments, the storage is for at least 11 months. In some embodiments, the storage is for at least 1 year. In some embodiments, the storage is for at least 2 years. In some embodiments, the storage is for at least 3 years. In some embodiments, the storage is for at least 4 years. In some embodiments, the storage is for at least 5 or more years.

In various embodiments, the storage comprises one or more freeze-thaw cycles. In some embodiments, the storage comprises at least 1 freeze-thaw cycle. In some embodiments, the storage comprises at least 2 freeze-thaw cycles. In some embodiments, the storage comprises at least 3 freeze-thaw cycles. In some embodiments, the storage comprises at least 4 freeze-thaw cycles. In some embodiments, the storage comprises at least 5 freeze-thaw cycles. In some embodiments, the one or more freeze-thaw cycles comprise freezing the composition at about −65° or less for about 1.5 hours or more, and thawing at 30° C. for 1.5 hours.

In any of the embodiments contemplated herein, the composition is frozen.

### D. Methods

Also contemplated herein are methods of storage, cryopreservation, transduction, and expression of a transgene.

In one aspect, methods for storing a viral vector are provided comprising providing a viral vector, contacting the viral vector with any of the compositions contemplated herein, and storing the viral composition at a temperature of about 25° C. or lower. In some embodiments the storage temperature is 2-8° C. or lower. In some embodiments the storage temperature is 0° C. or lower.

In another aspect, a method for cryopreserving a viral vector is provided, comprising providing a viral vector, contacting the viral vector with any one of the compositions contemplated herein, freezing the viral composition, and storing the viral composition at a temperature of about 0° C. or lower. In some embodiments, the composition is stored at −20° C. or lower. In some embodiments, the composition is stored at −65° C. or lower. In some embodiments, the composition is stored at −80° C. or lower.

In various embodiments, the storage is for at least 24 hours. In some embodiments, the storage is for at least 48 hours. In some embodiments, the storage is for at least 72 hours. In some embodiments, the storage is for at least 96 hours. In some embodiments, the storage is for at least 120 hours. In some embodiments, the storage is for at least 144 hours. In some embodiments, the storage is for at least 168 hours. In some embodiments, the storage is for at least 1 week. In some embodiments, the storage is for at least 2 weeks. In some embodiments, the storage is for at least 3 weeks. In some embodiments, the storage is for at least 4 weeks. In some embodiments, the storage is for at least 1 month. In some embodiments, the storage is for at least 2 months. In some embodiments, the storage is for at least 3 months. In some embodiments, the storage is for at least 4 months. In some embodiments, the storage is for at least 5 months. In some embodiments, the storage is for at least 6 months. In some embodiments, the storage is for at least 7 months. In some embodiments, the storage is for at least 8 months. In some embodiments, the storage is for at least 9 months. In some embodiments, the storage is for at least 10 months. In some embodiments, the storage is for at least 11 months. In some embodiments, the storage is for at least 1 year. In some embodiments, the storage is for at least 2 years.

In another aspect, a method of transduction is provided, comprising contacting a cell with any one of the compositions comprising a viral vector contemplated herein, thereby transducing the cell.

In another aspect, a method of expressing a transgene in a cell is provided, comprising contacting a cell with any one of the compositions comprising a viral vector contemplated herein, wherein the viral vector comprises a transgene.

In various embodiments the cell is a mammalian cell. In some embodiments, the cell is a hematopoietic cell. In some embodiments, the cell is a hematopoietic stem or progenitor cell. In some embodiments, the cell is a CD34⁺ hematopoietic stem or progenitor cell. In some embodiments, the cell is a human CD34⁺ hematopoietic stem or progenitor cell.

In some embodiments, the cell is a peripheral blood mononuclear cell (PMBC). In some embodiments, the cell is a T cell. In some embodiments, the cell is an aβ T cell. In some embodiments, the cell is a γδ T cell. In some embodiments, the cell is a CD3⁺, CD4⁺, and/or CD8⁺ cell. In some embodiments, the cell is an immune effector cell. In some embodiments, the cell is a cytotoxic T lymphocyte (CTL), a tumor infiltrating lymphocyte (TIL), or a helper T cell. In some embodiments, the cell is a natural killer (NK) cell or natural killer T (NKT) cell.

An “immune effector cell,” is any cell of the immune system that has one or more effector functions (e.g., cytotoxic cell killing activity, secretion of cytokines, induction of ADCC and/or CDC). Illustrative immune effector cells contemplated herein are T lymphocytes, including but not limited to cytotoxic T cells (CTLs; CD8⁺ T cells), TILs, and helper T cells (HTLs; CD4⁺ T cells. In a particular embodiment, the cells comprise aβ T cells. In a particular embodiment, the cells comprise γδ T cells modified to express an aβ TCR. In one embodiment, immune effector cells include natural killer (NK) cells. In one embodiment, immune effector cells include natural killer T (NKT) cells.

Immune effector cells can be autologous/autogeneic (“self”) or non-autologous (“non-self,” e.g., allogeneic, syngeneic or xenogeneic). “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison. In preferred embodiments, the cells are autologous.

Illustrative immune effector cells used with the methods contemplated in particular embodiments include T lymphocytes. The terms “T cell” or “T lymphocyte” are art-recognized and are intended to include thymocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4⁺ T cell) CD4⁺ T cell, a cytotoxic T cell (CTL; CD8⁺ T cell), CD4⁺CD8⁺ T cell, CD4 CD8″ T cell, or any other subset of T cells. Other illustrative populations of T cells suitable for use in particular embodiments include naïve T cells (TN), T memory stem cells (TSCM), central memory T cells (TCM), effector memory T cells (TEM), and effector T cells (TEFF).

As would be understood by the skilled person, other cells may also be used as immune effector cells with the methods contemplated herein. In particular, immune effector cells also include NK cells, NKT cells, neutrophils, and macrophages. Immune effector cells also include progenitors of effector cells wherein such progenitor cells can be induced to differentiate into an immune effector cell in vivo or in vitro. Thus, in particular embodiments, immune effector cell includes progenitors of immune effectors cells such as hematopoietic stem cells (HSCs) contained within the CD34⁺ population of cells derived from cord blood, bone marrow or mobilized peripheral blood which upon administration in a subject differentiate into mature immune effector cells, or which can be induced in vitro to differentiate into mature immune effector cells.

The term, “CD34⁺ cell,” as used herein refers to a cell expressing the CD34 protein on its cell surface. “CD34,” as used herein refers to a cell surface glycoprotein (e.g., sialomucin protein) that often acts as a cell-cell adhesion factor and is involved in T cell entrance into lymph nodes. The CD34⁺ cell population contains hematopoietic stem cells (HSC), which upon administration to a patient differentiate and contribute to all hematopoietic lineages, including T cells, NK cells, NKT cells, neutrophils and cells of the monocyte/macrophage lineage.

Methods contemplated herein for making immune effector cells that express a transgene or therapeutic protein are provided in particular embodiments. In one embodiment, the method comprises transducing immune effector cells isolated from an individual such that the immune effector cells express the transgene or therapeutic protein, with a viral vector composition (e.g., a lentiviral composition) contemplated herein. In particular embodiments, the transduced cells are subsequently cultured for expansion, prior to administration to a subject.

In certain embodiments, the immune effector cells are isolated from an individual and genetically modified without further manipulation in vitro. Such cells can then be directly re-administered into the individual. In further embodiments, the immune effector cells are first activated and stimulated to proliferate in vitro prior to being genetically modified to express a transgene, e.g., an engineered a chimeric receptor (e.g., CAR, CCR, DARIC, or switch receptor), TCR, or other transgene (e.g., protein or cytokine). In this regard, the immune effector cells may be cultured before and/or after being genetically modified.

In particular embodiments, prior to in vitro manipulation or genetic modification of the immune effector cells described herein, the source of cells is obtained from a subject. In particular embodiments, modified immune effector cells comprise T cells.

In particular embodiments, PBMCs may be directly genetically modified to express a transgene or therapeutic protein. In certain embodiments, after isolation of PBMC, T lymphocytes are further isolated and in certain embodiments, both cytotoxic and helper T lymphocytes can be sorted into naïve, memory, and effector T cell subpopulations either before or after genetic modification and/or expansion.

The immune effector cells, such as T cells, can be genetically modified following isolation using known methods, or the immune effector cells can be activated and expanded (or differentiated in the case of progenitors) in vitro prior to being genetically modified. In a particular embodiment, the immune effector cells, such as T cells, are activated and stimulated for expansion and then genetically modified with a transgene or therapeutic protein (e.g., transduced with a viral vector composition contemplated herein). In various embodiments, T cells can be activated and expanded before or after genetic modification, using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; 7,572,631; and U.S. Patent Application Publication No. 20170051252.

In one embodiment, CD34⁺ hematopoietic stem or progenitor cells are transduced with a nucleic acid construct contemplated herein. In certain embodiments, the transduced CD34⁺ cells differentiate into mature immune effector cells in vivo following administration into a subject, generally the subject from whom the cells were originally isolated. In another embodiment, CD34⁺ cells may be stimulated in vitro prior to exposure to or after being genetically modified with one or more of the following cytokines: Flt-3 ligand (FLT3), stem cell factor (SCF), megakaryocyte growth and differentiation factor (TPO), IL-3 and IL-6 according to the methods described previously (Asheuer et al., 2004; Imren, et al., 2004).

In certain embodiments, the cells are transduced with a vector as contemplated herein in the presence of a polycationic polymer. In some embodiments, the polycationic polymer is polybrene, protamine sulfate, polyethylenimine, or a polyethylene glycol/poly-L-lysine block copolymer. In some embodiments, the cells are transduced in the presence of polybrene. In some embodiments, the cells are transduced in the presence of about 2 μg/ml polybrene. In some embodiments, the cells are transduced in the presence of about 3 μg/ml polybrene. In some embodiments, the cells are transduced in the presence of about 4 μg/ml polybrene. In some embodiments, the cells are transduced in the presence of about 5 μg/ml polybrene. In some embodiments, the cells are transduced in the presence of about 6 μg/ml polybrene. In some embodiments, the cells are transduced in the presence of about 7 μg/ml polybrene. In some embodiments, the cells are transduced in the presence of about 8 μg/ml polybrene. In some embodiments, the cells are transduced in the presence of about 2 μg/ml to about 8 μg/ml polybrene. In some embodiments, the cells are transduced in the presence of about 3 μg/ml to about 8 μg/ml polybrene. In some embodiments, the cells are transduced in the presence of about 4 μg/ml to about 8 μg/ml polybrene. In some embodiments, the cells are transduced in the presence of about 5 μg/ml to about 8 μg/ml polybrene. In some embodiments, the cells are transduced in the presence of about 6 μg/ml to about 8 μg/ml polybrene. In some embodiments, the cells are transduced in the presence of about 7 μg/ml to about 8 μg/ml polybrene.

### E. Viral Vectors

The compositions and methods contemplated herein comprise viral vectors. The term “vector” is used herein to refer to a nucleic acid molecule capable of transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. In particular embodiments, the vector is a viral vector or a non-viral vector.

As will be evident to one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). The term “viral vector” or “lentiviral vector” may refer either to a virus or viral particle capable of transferring a nucleic acid into a cell or to the transferred nucleic acid itself. Viral vectors and transfer plasmids contain structural and/or functional genetic elements that are primarily derived from a virus.

Illustrative examples of viral vector systems suitable for use in particular embodiments contemplated in particular embodiments include, but are not limited to, adeno-associated virus (AAV), retrovirus, herpes simplex virus (HSV), adenovirus, and vaccinia virus vectors.

In various embodiments, the vector is an adenoviral vector, an adeno-associated viral (AAV) vector, a herpes virus vector, a vaccinia virus vector, or a retroviral vector.

AAV is a small (˜26 nm) replication-defective, primarily episomal, non-enveloped virus. AAV can infect both dividing and non-dividing cells and may incorporate its genome into that of the host cell. Recombinant AAV (rAAV) are typically composed of, at a minimum, a transgene and its regulatory sequences, and 5′ and 3′ AAV inverted terminal repeats (ITRs). The ITR sequences are about 145 bp in length. In particular embodiments, the rAAV comprises ITRs and capsid sequences isolated from AAV1, AAV2, AAV3, AAV4, AAV5, AAV6, AAV7, AAV8, AAV9, or AAV10.

In some embodiments, a chimeric rAAV is used. In some embodiments, the rAAV comprises one or more ITR sequences. In some embodiments, the ITR sequences are isolated from one AAV serotype and the capsid sequences are isolated from a different AAV serotype. For example, a rAAV with ITR sequences derived from AAV2 and capsid sequences derived from AAV6 is referred to as AAV2/AAV6. In particular embodiments, the rAAV vector may comprise ITRs from AAV2, and capsid proteins from any one of AAV1, AAV2, AAV3, AAV4, AAV5, AAV6, AAV7, AAV8, AAV9, or AAV10. In a preferred embodiment, the rAAV comprises ITR sequences derived from AAV2 and capsid sequences derived from AAV6. In a preferred embodiment, the rAAV comprises ITR sequences derived from AAV2 and capsid sequences derived from AAV2.

In some embodiments, engineering and selection methods can be applied to AAV capsids to make them more likely to transduce cells of interest.

Construction of rAAV vectors, production, and purification thereof have been disclosed, e.g., in U.S. Pat. Nos. 9,169,494; 9,169,492; 9,012,224; 8,889,641; 8,809,058; and 8,784,799, each of which is incorporated by reference herein, in its entirety.

As used herein, the term “retrovirus” refers to an RNA virus that reverse transcribes its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

In some embodiments, the viral vector is a lentiviral vector. As used herein, the term “lentivirus” refers to a group (or genus) of complex retroviruses. Illustrative lentiviruses include, but are not limited to: HIV (human immunodeficiency virus; including HIV type 1, and HIV 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred.

In some embodiments, the lentiviral vector is derived from human immunodeficiency cirus-1 (HIV-1) or human immunodeficiency virus 2 (HIV-2). In particular embodiments, the lentiviral vector is derived from human immunodeficiency cirus-1 (HIV-1).

In various embodiments, a lentiviral vector contemplated herein comprises one or more LTRs, and one or more, or all, of the following accessory elements: a cPPT/FLAP, a Psi (I) packaging signal, an export element, poly (A) sequences, and may optionally comprise a WPRE or HPRE, an insulator element, a selectable marker, and a cell suicide gene, as discussed elsewhere herein.

In particular embodiments, lentiviral vectors contemplated herein may be integrative or non-integrating or integration defective lentivirus. As used herein, the term “integration defective lentivirus” or “IDLV” refers to a lentivirus having an integrase that lacks the capacity to integrate the viral genome into the genome of the host cells. Integration-incompetent viral vectors have been described in patent application WO 2006/010834, which is herein incorporated by reference in its entirety.

Illustrative mutations in the HIV-1 pol gene suitable to reduce integrase activity include, but are not limited to: H12N, H12C, H16C, H16V, S81R, D41A, K42A, H51A, Q53C, D55V, D64E, D64V, E69A, K71A, E85A, E87A, D116N, D116I, D116A, N120G, N120I, N120E, E152G, E152A, D35E, K156E, K156A, E157A, K159E, K159A, K160A, R166A, D167A, E170A, H171A, K173A, K186Q, K186T, K188T, E198A, R199c, R199T, R199A, D202A, K211A, Q214L, Q216L, Q221 L, W235F, W235E, K236S, K236A, K246A, G247W, D253A, R262A, R263A and K264H.

In one embodiment, the HIV-1 integrase deficient pol gene comprises a D64V, D116I, D116A, E152G, or E152A mutation; D64V, D116I, and E152G mutations; or D64V, D116A, and E152A mutations.

In one embodiment, the HIV-1 integrase deficient pol gene comprises a D64V mutation.

The term “long terminal repeat (LTR)” refers to domains of base pairs located at the ends of retroviral DNAs which, in their natural sequence context, are direct repeats and contain U3, R and U5 regions.

As used herein, the term “FLAP element” or “cPPT/FLAP” refers to a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173. In another embodiment, a lentiviral vector contains a FLAP element with one or more mutations in the cPPT and/or CTS elements. In yet another embodiment, a lentiviral vector comprises either a cPPT or CTS element. In yet another embodiment, a lentiviral vector does not comprise a cPPT or CTS element.

As used herein, the term “packaging signal” or “packaging sequence” refers to psi [Ψ] sequences located within the retroviral genome which are required for insertion of the viral RNA into the viral capsid or particle, see e.g., Clever et al., 1995. J. of Virology, Vol. 69, No. 4; pp. 2101-2109.

The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65:1053; and Cullen et al., 1991. Cell 58:423), and the hepatitis B virus post-transcriptional regulatory element (HPRE).

In particular embodiments, expression of heterologous sequences in viral vectors is increased by incorporating posttranscriptional regulatory elements, efficient polyadenylation sites, and optionally, transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766).

Lentiviral vectors preferably contain several safety enhancements as a result of modifying the LTRs. “Self-inactivating” (SIN) vectors refers to replication-defective vectors, e.g., in which the right (3′) LTR enhancer-promoter region, known as the U3 region, has been modified (e.g., by deletion or substitution) to prevent viral transcription beyond the first round of viral replication. An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters.

The terms “pseudotype” or “pseudotyping” as used herein, refer to a virus that has viral envelope proteins that have been substituted with those of another virus possessing preferable characteristics. For example, HIV can be pseudotyped with vesicular stomatitis virus G-protein (VSV-G) envelope proteins, which allows HIV to infect a wider range of cells because HIV envelope proteins (encoded by the env gene) normally target the virus to CD4⁺ presenting cells. Other envelope proteins for pseudotyping include, but are not limited to, envelopes from other vesiculovirus strains (e.g., Indiana, Alagoas, New Jersey, Isfahan, CoCal, Maraba, or Piry), Measles envelope proteins, Sindbis envelope proteins, Morbillivirus proteins (e.g., F and H Proteins), Sendai proteins (e.g., F and HN proteins), or Paramyxoviridae proteins (e.g., F and H proteins).

In certain embodiments, lentiviral vectors are produced according to known methods. See e.g., Kutner et al., BMC Biotechnol. 2009; 9:10. doi: 10.1186/1472-6750-9-10; Kutner et al. Nat. Protoc. 2009; 4 (4): 495-505. doi: 10.1038/nprot.2009.22, and WO2023/003844.

According to certain specific embodiments contemplated herein, most or all of the viral vector backbone sequences are derived from a lentivirus, e.g., HIV-1. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. Moreover, a variety of lentiviral vectors are known in the art, see Naldini et al., (1996a, 1996b, and 1998); Zufferey et al., (1997); Dull et al., 1998, U.S. Pat. Nos. 6,013,516; and 5,994,136, many of which may be adapted to produce a viral vector or transfer plasmid contemplated herein.

In various embodiments, the viral vector is a lentiviral vector. In some embodiments, the lentiviral vector is an AnkT9W vector Lenti-D vector. In some embodiments, the lentiviral vector is an AnkT9W vector, a T9Ank2W vector, a TNS9 vector, a TNS9.3 vector, a TNS9.3.55 vector, a lentiglobin HPV569 vector, a lentiglobin BB305 vector, a BG-1 vector, a BGM-1 vector, a GLOBE vector, a G-GLOBE vector, a βAS3-FB vector, or a derivative thereof. In some embodiments, the lentiviral vector is an AnkT9W vector or a derivative thereof. In some embodiments, the lentiviral vector is a T9Ank2W vector or a derivative thereof. In some embodiments, the lentiviral vector is a TNS9 vector or a derivative thereof. In some embodiments, the lentiviral vector is a TNS9.3 vector or a derivative thereof. In some embodiments, the lentiviral vector is a TNS9.3.55 vector or a derivative thereof. In some embodiments, the lentiviral vector is a lentiglobin HPV569 vector or a derivative thereof. In some embodiments, the lentiviral vector is a lentiglobin BB305 vector or a derivative thereof. In some embodiments, the lentiviral vector is a BG-1 vector or a derivative thereof. In some embodiments, the lentiviral vector is a BGM-1 vector or a derivative thereof. In some embodiments, the lentiviral vector is a GLOBE vector or a derivative thereof. In some embodiments, the lentiviral vector is a G-GLOBE vector or a derivative thereof. In some embodiments, the lentiviral vector is a βAS3-FB vector or a derivative thereof. In particular embodiments, the lentiviral vector is BB305.

### F. Transgenes

In any of the embodiments contemplated herein, the viral vector comprises a polynucleotide comprising a transgene. In some embodiments, the transgene encodes a therapeutic protein.

As used herein, the term “transgene” refers to an exogenous nucleic acid sequence that encodes a protein or functional nucleotide. The transgene may encode for a non-natural or naturally occurring protein or polypeptide. As used herein, the term “therapeutic protein” refers to a protein or polypeptide encoded by a transgene useful for the treatment of a disease in a patient (e.g., cancer).

In various embodiments, the transgene or therapeutic protein is a chimeric antigen receptor (CAR), a chimeric costimulatory receptor (CCR), an aβ T cell receptor (αβ-TCR), a γδ T cell receptor (γδ-TCR), a dimerizing agent regulated immunoreceptor complex (DARIC), or switch receptor that specifically binds a target antigen. In some embodiments, the transgene or therapeutic protein is an exogenous costimulatory factor, immunomodulatory factor, agonist for a costimulatory factor, antagonist for an immunosuppressive factor, immune cell engager, or fusion protein. In some embodiments, the transgene or therapeutic protein is a costimulatory factor. In some embodiments, the transgene or therapeutic protein is a cytokine.

The terms “specific binding affinity” or “specifically binds” or “specifically bound” or “specific binding” or “specifically targets” as used herein, describe binding of binding domain to a target antigen at greater binding affinity than background binding. A binding domain “specifically binds” to a target antigen, if it binds to or associates with the antigen with an affinity or Kₐ (i.e., an equilibrium association constant of a particular binding interaction with units of 1/M) of, for example, greater than or equal to about 10⁵ M⁻¹. In certain embodiments, a binding domain (or a fusion protein comprising the same) binds to a target with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹ “High affinity” binding domains (or single chain fusion proteins thereof) refer to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater.

The terms “selectively binds” or “selectively bound” or “selectively binding” or “selectively targets” and describe preferential binding of one molecule to a target molecule (on-target binding) in the presence of a plurality of off-target molecules.

In various embodiments, the CAR, CCR, DARIC, or switch receptor specifically binds a target antigen selected from the group consisting of: alpha folate receptor (FRα), αᵥβ₆ integrin, B cell maturation antigen (BCMA), B7-H3 (CD276), B7-H₆, carbonic anhydrase IX (CA1X), CD16, CD19, CD20, CD22, CD30, CD33, CD37, CD38, CD44, CD44v6, CD44v7/8, CD70, CD79a, CD79b, CD123, CD133, CD138, CD171, carcinoembryonic antigen (CEA), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC₃), EGFR family including ErbB2 (HER2), IL-10Rα, IL-13Rα2, Kappa, cancer/testis antigen 2 (LAGE-1A), K-Ras, K-Ras G12C, K-Ras G12D, Lambda, Lewis-Y (LeY), L1 cell adhesion molecule (L1-CAM), melanoma antigen gene (MAGE)-A1, MAGE-A3, MAGE-A4, MAGE-A6, MAGEA10, melanoma antigen recognized by T cells 1 (MelanA or MART1), Mesothelin (MSLN), MUC1, MUC16, MHC class I chain related proteins A (MICA), MHC class I chain related proteins B (MICB), neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), tumor endothelial marker 1 (TEM1/CD248), tumor endothelial marker 7-related (TEM7R), trophoblast glycoprotein (TPBG), UL16-binding protein (ULBP) 1, ULBP2, ULBP3, ULBP4, ULBP5, ULBP6, vascular endothelial growth factor receptor 2 (VEGFR2), and Wilms tumor 1 (WT-1).

In various embodiments, the CAR, CCR, or DARIC, specifically binds a target antigen selected from the group consisting of: BCMA, CD33, CD20, CD79a, CD79b, CLL-1, IGF2BP3/A3, MUC16, NY-ESO, PRAME, PSA, TACI, and TP53.

In various embodiments, the αβ-TCR or γδ-TCR specifically binds a target antigen selected from the group consisting of: α-fetoprotein (AFP), B Melanoma Antigen (BAGE) family members, Brother of the regulator of imprinted sites (BORIS), Cancer-testis antigens, Cancer-testis antigen 83 (CT-83), Carbonic anhydrase IX (CA1X), Carcinoembryonic antigen (CEA), Cytomegalovirus (CMV) antigens, Cytotoxic T cell (CTL)-recognized antigen on melanoma (CAMEL), Epstein-Barr virus (EBV) antigens, G antigen 1 (GAGE-1), GAGE-2, GAGE-3, GAGE-4, GAGE-5, GAGE-6, GAGE-7B, GAGE-8, Glycoprotein 100 (GP100), Hepatitis B virus (HBV) antigens, Hepatitis C virus (HCV) non-structure protein 3 (NS3), Human Epidermal Growth Factor Receptor 2 (HER-2), Human papillomavirus (HPV)-E6, HPV-E7, Human telomerase reverse transcriptase (hTERT), K-Ras, K-Ras G12C, K-Ras G12D, Latent membrane protein 2 (LMP2), Melanoma antigen family A, 1 (MAGE-A1), MAGE-A2, MAGE-A3, MAGE-A4, MAGE-A6, MAGE-A10, MAGE-A12, Melanoma antigen recognized by T cells (MART-1), Mesothelin (MSLN), Mucin 1 (MUC1), Mucin 16 (MUC16), New York esophageal squamous cell carcinoma-1 (NYESO-1), P53, P antigen (PAGE) family members, Placenta-specific 1 (PLAC1), Preferentially expressed antigen in melanoma (PRAME), Survivin, Synovial sarcoma X 1 (SSX1), Synovial sarcoma X 2 (SSX2), Synovial sarcoma X 3 (SSX3), Synovial sarcoma X 4 (SSX4), Synovial sarcoma X 5 (SSX5), Synovial sarcoma X 8 (SSX8), Thyroglobulin, Tyrosinase, Tyrosinase related protein (TRP) 1, TRP2, Wilms tumor protein (WT-1), X Antigen Family Member 1 (XAGE1), and X Antigen Family Member 2 (XAGE2).

In various embodiments, the αβ-TCR or γδ-TCR specifically binds a MAGE-A4 peptide.

Illustrative transgenes, therapeutic proteins, including but not limited to CARs, CCRs, αβ-TCRs, γδ-TCRs, DARICs, and switch receptors, and methods of making and using the same are disclosed in any one or more of WO2021/067347, WO2020/252110, WO2020/227474, WO2020/227475, WO2020/227481, WO2020/193767, WO2020/123947, WO2019/126724, WO2018/094244, WO2017/180993, WO2016094304, WO2015017214, WO2013154760, which are incorporated by reference herein, in their entirety.

In various embodiments, the transgene or therapeutic protein is a therapeutic globin for treatment of a hemoglobinopathy or an ABCD1 gene for the treatment of CALD.

In certain embodiments, the transgene or therapeutic protein is a globin. In some embodiments, the globin is a human β-globin, a human δ-globin, an anti-sickling globin, a human γ-globin, a human β^{A-T87Q}-globin, a human β^{A-G16D/E22A/T87Q}-globin, or a human β^{A-T87Q/K95E/K120E}-globin protein. In certain embodiments, the globin is a human β-globin protein. In certain embodiments, the globin is an anti-sickling globin protein. In certain embodiments, the globin is a human γ-globin protein. In certain embodiments, the globin is a human β^{A-T87Q}-globin protein. In certain aspects, the globin is a human β^{A-G16D/E22A/T87Q}-globin protein. In certain aspects, the globin is a human β^{A-T87Q/K95E/K120E}-globin protein. In certain embodiments, the β-globin is a human β-globin. In particular embodiments, the β-globin is β^{A-T87Q} globin.

In particular embodiments, polynucleotides encoding a transgene or therapeutic protein as described herein, are provided.

As used herein, the terms “polynucleotide” or “nucleic acid” refer to deoxyribonucleic acid (DNA), ribonucleic acid (RNA) and DNA/RNA hybrids. Polynucleotides may be single-stranded or double-stranded and either recombinant, synthetic, or isolated. Polynucleotides include, but are not limited to: pre-messenger RNA (pre-mRNA), messenger RNA (mRNA), RNA, short interfering RNA (siRNA), short hairpin RNA (shRNA), microRNA (miRNA), ribozymes, genomic RNA (gRNA), plus strand RNA (RNA(+)), minus strand RNA (RNA(−)), tracrRNA, crRNA, single guide RNA (sgRNA), synthetic RNA, synthetic mRNA, genomic DNA (gDNA), PCR amplified DNA, complementary DNA (cDNA), synthetic DNA, or recombinant DNA. Polynucleotides refer to a polymeric form of nucleotides of at least 5, at least 10, at least 15, at least 20, at least 25, at least 30, at least 40, at least 50, at least 100, at least 200, at least 300, at least 400, at least 500, at least 1000, at least 5000, at least 10000, or at least 15000 or more nucleotides in length, either ribonucleotides or deoxyribonucleotides or a modified form of either type of nucleotide, as well as all intermediate lengths. It will be readily understood that “intermediate lengths,” in this context, means any length between the quoted values, such as 6, 7, 8, 9, etc., 101, 102, 103, etc.; 151, 152, 153, etc.; 201, 202, 203, etc. In particular embodiments, polynucleotides or variants have at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference sequence.

As used herein, “isolated polynucleotide” refers to a polynucleotide that has been purified from the sequences which flank it in a naturally-occurring state, e.g., a DNA fragment that has been removed from the sequences that are normally adjacent to the fragment. In particular embodiments, an “isolated polynucleotide” also refers to a complementary DNA (cDNA), a recombinant DNA, or other polynucleotide that does not exist in nature and that has been made by the hand of man. In particular embodiments, an isolated polynucleotide is a synthetic polynucleotide, a semi-synthetic polynucleotide, or a polynucleotide obtained or derived from a recombinant source.

In various embodiments, a polynucleotide comprises an mRNA encoding a polypeptide contemplated herein. In certain embodiments, the mRNA comprises a cap, one or more nucleotides, and a poly (A) tail.

In particular embodiments, polynucleotides described herein, including polynucleotides encoding a transgene or therapeutic protein as described herein, may be codon-optimized. As used herein, the term “codon-optimized” refers to substituting codons in a polynucleotide encoding a polypeptide in order to increase the expression, stability and/or activity of the polypeptide. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, (x) systematic variation of codon sets for each amino acid, and/or (xi) isolated removal of spurious translation initiation sites.

As used herein the term “nucleotide” refers to a heterocyclic nitrogenous base in N-glycosidic linkage with a phosphorylated sugar. Nucleotides are understood to include natural bases, and a wide variety of art-recognized modified bases. Such bases are generally located at the l′ position of a nucleotide sugar moiety. Nucleotides generally comprise a base, sugar and a phosphate group. In ribonucleic acid (RNA), the sugar is a ribose, and in deoxyribonucleic acid (DNA) the sugar is a deoxyribose, i.e., a sugar lacking a hydroxyl group that is present in ribose.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion, substitution, or modification of at least one nucleotide. Accordingly, the terms “polynucleotide variant” and “variant” include polynucleotides in which one or more nucleotides have been added or deleted, or modified, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 86%, 97%, 98%, or 99% sequence identity to any of the reference sequences described herein.

The term “nucleic acid cassette” or “expression cassette” as used herein refers to genetic sequences within the vector which can express an RNA, and subsequently a polypeptide. In one embodiment, the nucleic acid cassette contains a gene(s)-of-interest, e.g., a polynucleotide(s)-of-interest. In another embodiment, the nucleic acid cassette contains one or more expression control sequences, e.g., a promoter, enhancer, poly (A) sequence, and a gene(s)-of-interest, e.g., a polynucleotide(s)-of-interest. Vectors may comprise 1, 2, 3, 4, 5, 6, 7, 8, 9 or 10 or more nucleic acid cassettes. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. The cassette can be removed and inserted into a plasmid or viral vector as a single unit.

Polynucleotides include polynucleotide(s)-of-interest. As used herein, the term “polynucleotide-of-interest” refers to a polynucleotide encoding a polypeptide or fusion polypeptide or a polynucleotide that serves as a template for the transcription of an inhibitory polynucleotide, as contemplated herein.

The polynucleotides contemplated herein, regardless of the length of the coding sequence itself, may be combined with other DNA sequences, such as promoters and/or enhancers, untranslated regions (UTRs), signal sequences, Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed, with the total length preferably being limited by the ease of preparation and use in the intended recombinant DNA protocol.

Polynucleotides can be prepared, manipulated, expressed and/or delivered using any of a variety of well-established techniques known and available in the art. In order to express a desired polypeptide, a nucleotide sequence encoding the polypeptide, can be inserted into appropriate vector.

Illustrative examples of vectors include, but are not limited to plasmid, autonomously replicating sequences, and transposable elements, e.g., Sleeping Beauty, PiggyBac.

Additional Illustrative examples of vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses.

Illustrative examples of viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40).

Illustrative examples of expression vectors include, but are not limited to, pClneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells. In particular embodiments, coding sequences of polypeptides disclosed herein can be ligated into such expression vectors for the expression of the polypeptides in mammalian cells.

“Expression control sequences,” “control elements,” or “regulatory sequences” present in an expression vector are those non-translated regions of the vector including an origin of replication, selection cassettes, promoters, enhancers, translation initiation signals (Shine Dalgarno sequence or Kozak sequence) introns, a polyadenylation sequence, 5′ and 3′ untranslated regions, all of which interact with host cellular proteins to carry out transcription and translation. Such elements may vary in their strength and specificity. Depending on the vector system and host utilized, any number of suitable transcription and translation elements, including ubiquitous promoters and inducible promoters may be used.

In particular embodiments, a polynucleotide comprises a vector, including but not limited to expression vectors and viral vectors. A vector may comprise one or more exogenous, endogenous, or heterologous control sequences such as promoters and/or enhancers. An “endogenous control sequence” is one which is naturally linked with a given gene in the genome. An “exogenous control sequence” is one which is placed in juxtaposition to a gene by means of genetic manipulation (i.e., molecular biological techniques) such that transcription of that gene is directed by the linked enhancer/promoter. A “heterologous control sequence” is an exogenous sequence that is from a different species than the cell being genetically manipulated. A “synthetic” control sequence may comprise elements of one more endogenous and/or exogenous sequences, and/or sequences determined in vitro or in silico that provide optimal promoter and/or enhancer activity for the particular therapy.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately to 30 bases upstream from the site where transcription is initiated and/or another sequence found 70 to 80 bases upstream from the start of transcription, a CNCAAT region where N may be any nucleotide.

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide-of-interest, wherein the expression control sequence directs transcription of the nucleic acid corresponding to the second sequence.

As used herein, the term “constitutive expression control sequence” refers to a promoter, enhancer, or promoter/enhancer that continually or continuously allows for transcription of an operably linked sequence. A constitutive expression control sequence may be a “ubiquitous” promoter, enhancer, or promoter/enhancer that allows expression in a wide variety of cell and tissue types or a “cell specific,” “cell type specific,” “cell lineage specific,” or “tissue specific” promoter, enhancer, or promoter/enhancer that allows expression in a restricted variety of cell and tissue types, respectively.

Illustrative ubiquitous expression control sequences suitable for use in particular embodiments include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EFla) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, d1587rev primer-binding site substituted (MND) U3 promoter (Haas et al. Journal of Virology. 2003; 77 (17): 9439-9450).

In one embodiment, a vector comprises an MNDU3 promoter. In one embodiment, a vector comprises an EF1a promoter comprising the first intron of the human EF1a gene. In one embodiment, a vector comprises an EF1a promoter that lacks the first intron of the human EF1a gene.

In a particular embodiment, it may be desirable to use a cell, cell type, cell lineage or tissue specific expression control sequence to achieve cell type specific, lineage specific, or tissue specific expression of a desired polynucleotide sequence (e.g., to express a particular nucleic acid encoding a polypeptide in only a subset of cell types, cell lineages, or tissues or during specific stages of development).

In a particular embodiment, it may be desirable to express a polynucleotide a T cell specific promoter.

As used herein, “conditional expression” may refer to any type of conditional expression including, but not limited to, inducible expression; repressible expression; expression in cells or tissues having a particular physiological, biological, or disease state, etc. This definition is not intended to exclude cell type or tissue specific expression. Certain embodiments provide conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc. Inducer agents include, but are not limited to glucocorticoids, estrogens, mifepristone (RU486), metals, interferons, small molecules, cumate, tetracycline, doxycycline, and variants thereof.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al., 1990. Trends Biochem Sci 15 (12): 477-83) and Jackson and Kaminski. 1995. RNA 1 (10): 985-1000. Examples of IRES generally employed by those of skill in the art include those described in U.S. Pat. No. 6,692,736. Further examples of “IRES” known in the art include, but are not limited to IRES obtainable from picornavirus (Jackson et al., 1990) and IRES obtainable from viral or cellular mRNA sources, such as for example, immunoglobulin heavy-chain binding protein (BiP), the vascular endothelial growth factor (VEGF) (Huez et al. 1998. Mol. Cell. Biol. 18 (11): 6178-6190), the fibroblast growth factor 2 (FGF-2), and insulin-like growth factor (IGFII), the translational initiation factor eIF4G and yeast transcription factors TFIID and HAP4, the encephelomycarditis virus (EMCV) which is commercially available from Novagen (Duke et al., 1992. J. Virol 66 (3): 1602-9) and the VEGF IRES (Huez et al., 1998. Mol Cell Biol 18 (11): 6178-90). IRES have also been reported in viral genomes of Picornaviridae, Dicistroviridae and Flaviviridae species and in HCV, Friend murine leukemia virus (FrMLV) and Moloney murine leukemia virus (MoMLV).

In one embodiment, the IRES used in polynucleotides contemplated herein is an EMCV IRES.

In particular embodiments, the polynucleotides comprise a consensus Kozak sequence. As used herein, the term “Kozak sequence” refers to a short nucleotide sequence that greatly facilitates the initial binding of mRNA to the small subunit of the ribosome and increases translation. The consensus Kozak sequence is (GCC) RCCATGG (SEQ ID NO: 198), where R is a purine (A or G) (Kozak, 1986. Cell. 44 (2): 283-92, and Kozak, 1987. Nucleic Acids Res. 15(20): 8125-48).

Elements directing the efficient termination and polyadenylation of the heterologous nucleic acid transcripts increases heterologous gene expression. Transcription termination signals are generally found downstream of the polyadenylation signal. In particular embodiments, vectors comprise a polyadenylation sequence 3′ of a polynucleotide encoding a polypeptide to be expressed. The term “polyA site” or “polyA sequence” as used herein denotes a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Cleavage and polyadenylation is directed by a poly (A) sequence in the RNA. The core poly (A) sequence for mammalian pre-mRNAs has two recognition elements flanking a cleavage-polyadenylation site. Typically, an almost invariant AAUAAA hexamer lies 20-50 nucleotides upstream of a more variable element rich in U or GU residues. Cleavage of the nascent transcript occurs between these two elements and is coupled to the addition of up to 250 adenosines to the 5′ cleavage product. In particular embodiments, the core poly (A) sequence is an ideal polyA sequence (e.g., AATAAA, ATTAAA, AGTAAA). In particular embodiments, the poly(A) sequence is an SV40 polyA sequence, a bovine growth hormone polyA sequence (BGHpA), a rabbit β-globin polyA sequence (rBgpA), variants thereof, or another suitable heterologous or endogenous polyA sequence known in the art. In particular embodiments, the poly(A) sequence is synthetic.

Polynucleotides encoding one or more polypeptides, or fusion polypeptides may be introduced into immune effector cells, e.g., T cells, by both non-viral and viral methods. In particular embodiments, delivery of one or more polynucleotides may be provided by the same method or by different methods, and/or by the same vector or by different vectors.

All publications, patent applications, and issued patents cited in this specification are herein incorporated by reference as if each individual publication, patent application, or issued patent were specifically and individually indicated to be incorporated by reference.

Although the foregoing embodiments have been described in some detail by way of illustration and example for purposes of clarity of understanding, it will be readily apparent to one of ordinary skill in the art in light of the teachings contemplated herein that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Lentiviral Vector Purification and Preparation for Screening

The base process for formulating lentiviral vector (LVV) begins with ultrafiltration (UF) and diafiltration (DF) (together UFDF) via tangential flow filtration (TFF) and is followed by dilution with 2×/1× stem cell growth media (SCGM). A hollow fiber membrane with a 500 kDa cutoff is used to concentrate purified LVV by 4-5 fold. The product is then exchanged into a diafiltration buffer of 50 mM HEPES (pH 7.5) and 100 mM NaCl. The resulting material is the UFDF pool. To achieve the control formulated product, the UFDF pool is diluted in a 1:1 weight ratio with 2×/1×SCGM. The formulated product is then filtered through a 0.22-micron polyethersulfone (PES) membrane to obtain the intermediate bulk. To achieve different formulated products for screening as described herein, the process remained the same as described except the diafiltration buffer was varied using different buffering agents and the UFDF pool was formulated in a 1:1 ratio with 2-fold concentrated formulation solution. After production, approximately 1 mL of intermediate bulk was filled into 2 mL plastic vials enclosed with rubber serum stoppers.

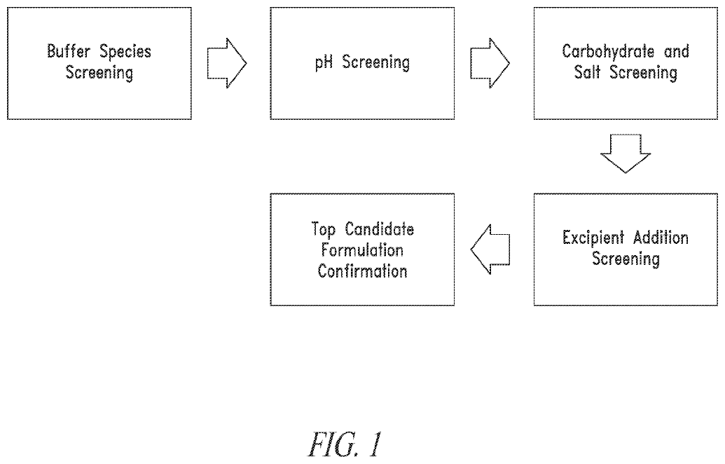

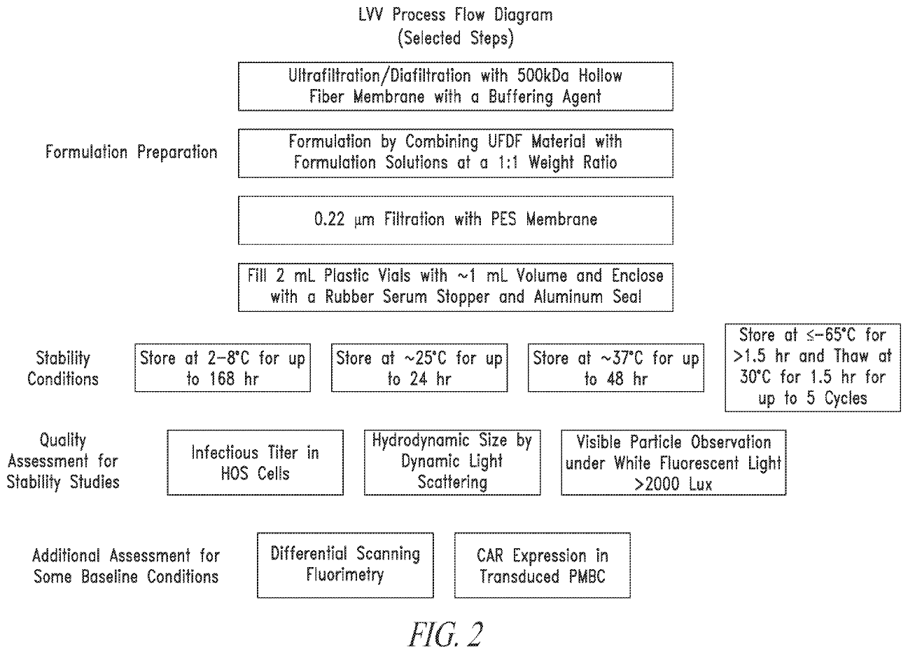

A broad overview of the screening process/steps and formula components tested are shown in FIG. 1. To determine the optimal formulation, the stability of the intermediate bulk was evaluated at expected or desired manufacturing conditions such as storage at 25° C. for up to 24 hours and 2-8° C. for up to 48 or 168 hours. These conditions were used for screening. Stress conditions were also evaluated to assess formulation robustness, including (i) up to five freeze-thaw cycles with freezing at ≤−65° C. for >1.5 hours and thawing at 30° C. for 1.5 hours, or (ii) storage at 37° C. for up to 48 hours (FIG. 2).

### Example 2

### Analysis of Lentiviral Cryopreservation Formulations

Stability was assessed by infectious titer measured by viral transduction of HOS cells, hydrodynamic size by dynamic light scattering (DLS), and the number of visible particles observed with a black or white background under fluorescent light. The unfolding temperature of some the formulations were evaluated by differential scanning fluorimetry (DSF). Formulations after storage at different temperature and/or freeze-thaw conditions were compared to the initial value or baseline. The formulations that maintained the integrity of LVV were further evaluated for CAR expression in PBMCs.

Infectious titer was used as the primary indicator of stability. Infectious titer or the number of transduction units was measured by viral transduction of HOS cells using standard techniques (see, e.g., GENE Therapy (2002), 9, 1155-1162). The selection criterion was a recovery above 75% (infectious titer after storage/starting infectious titer ×100%).

Changes in hydrodynamic size by dynamic light scattering (DLS) were noted when the size increased approximately 10 nm or more after storage.

The number of visible particles were monitored by visual observation under light with an output greater than 2000 Lux. The particles may be attributed to impurities such as host cell proteins (HCP) or lentivirus aggregation. The formulations were assessed to determine if particle free solutions could be maintained for extended durations. Appearance was monitored to distinguish inherent, intrinsic, and foreign particles post-vial fill.

DSF was used for characterizing the conformational stability of the lentivirus to differentiate formulations. A lower thermal unfolding temperature may indicate that the lentivirus is less stable in a particular formulation.

### Summary of Experimental Results for Buffer Species Screening

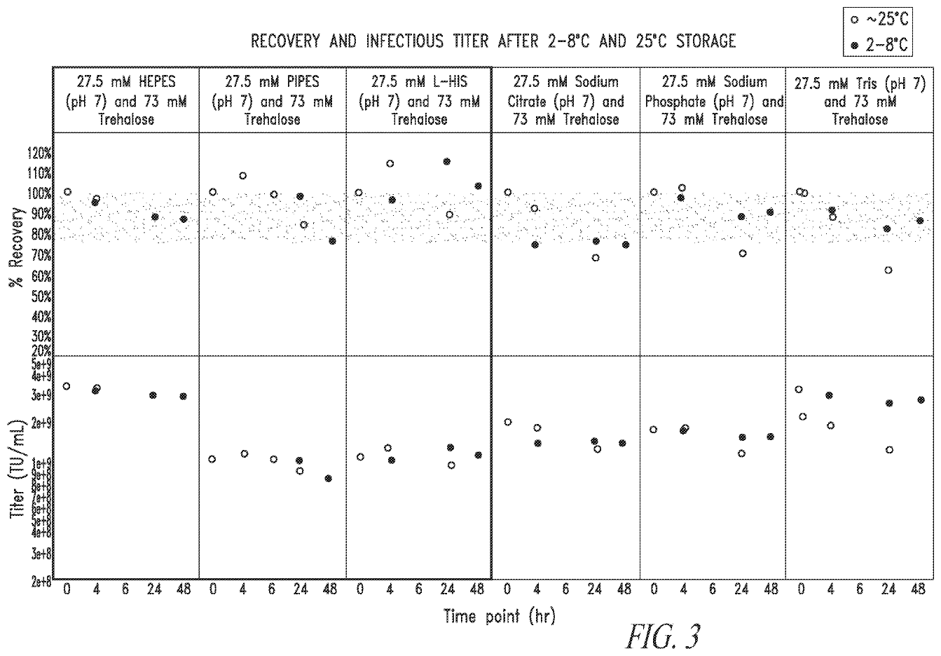

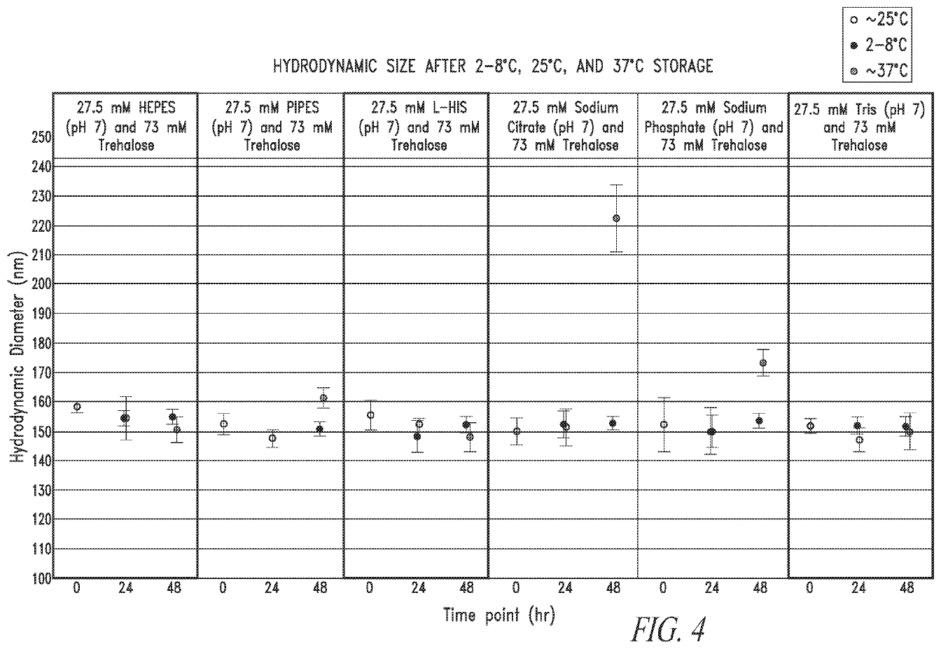

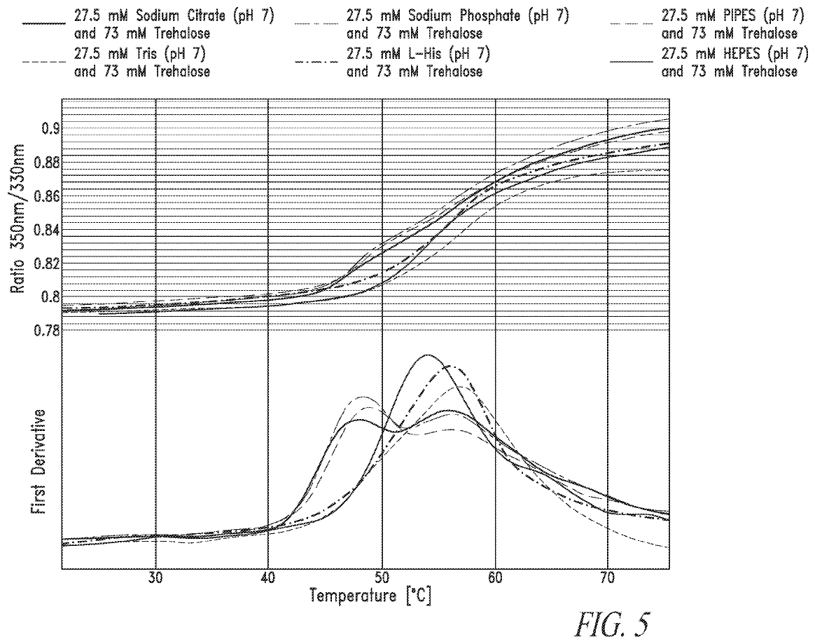

For selection of a buffering agent, the buffers evaluated were HEPES, sodium citrate, PIPES, L-histidine (L-His), sodium phosphate, and Tris at a concentration of 27.5 mM and pH 7. These formulations also contained 73 mM trehalose. Based on >75% infectious titer recovery in transduced HOS cells, the HEPES, PIPES, and L-His formulations had the least change after 25° C. storage for 24 hours and 2-8° C. storage for 48 hours (FIG. 3). By hydrodynamic size, no change in size was observed for any formulation after 25° C. storage for 24 hours and 2-8° C. storage for 48 hours. After storage at 37° C. for 48 hours, no change in size was observed for the HEPES, L-His, and Tris formulations, while the sodium citrate and sodium phosphate formulations had a change in size (FIG. 4). By thermal unfolding measurements, all buffering agents had comparable onset of unfolding ranging between 45-47° C. (FIG. 5). Sodium citrate, sodium phosphate, and PIPES appear to have two unfolding transitions, whereas L-His, HEPES, and Tris buffers appear to have one unfolding transition. Based on the first unfolding transition, L-His, HEPES, and Tris had the highest thermal unfolding temperature of 54-57° C., whereas sodium citrate, sodium phosphate, and PIPES had an unfolding temperature of 48-49° C.

### Summary of Experimental Results for pH Screening

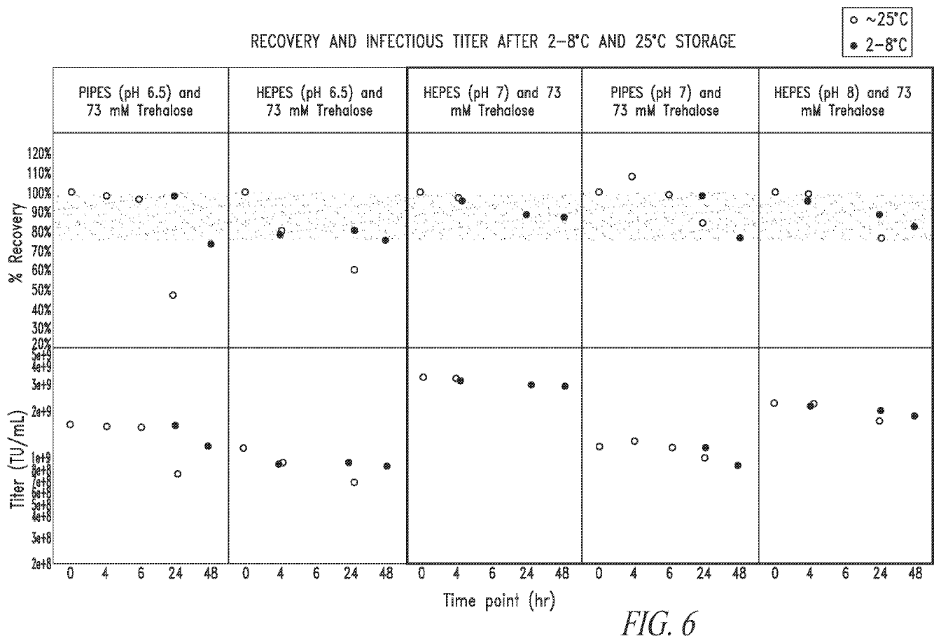

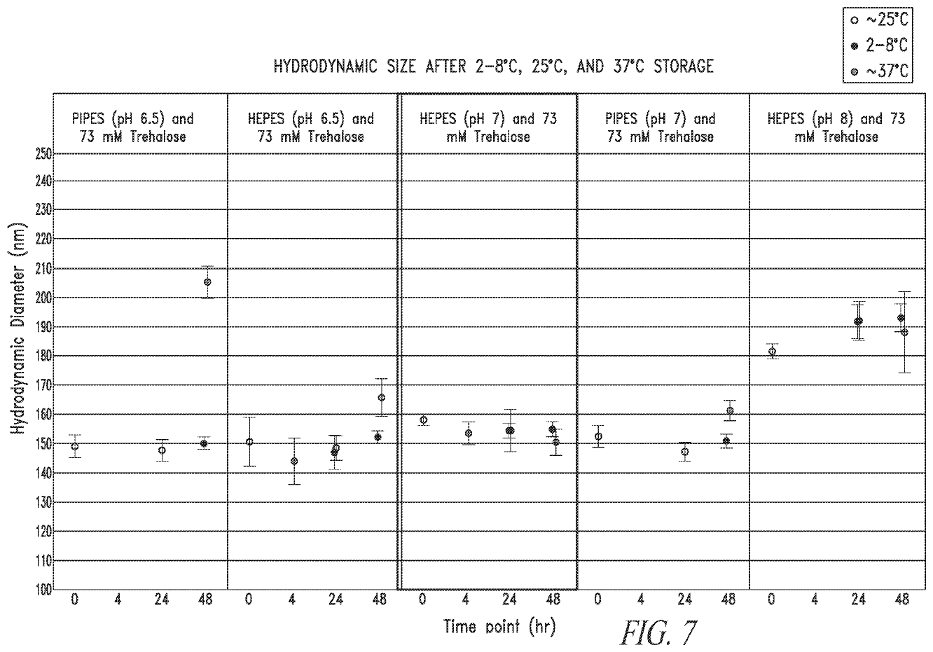

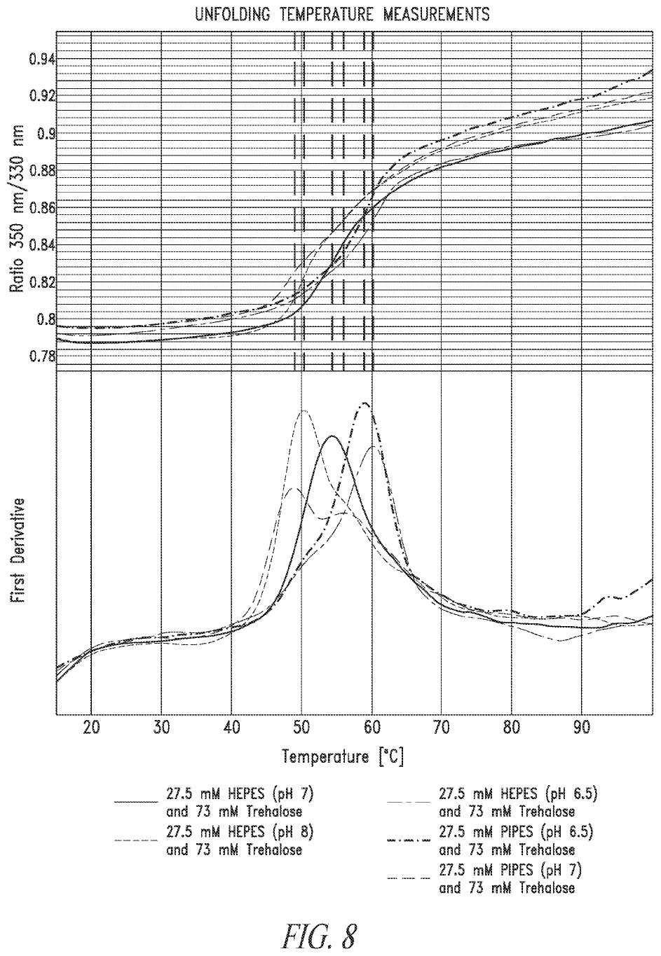

Target pH was assessed by evaluating LVV in 27.5 mM HEPES and 73 mM trehalose at pH 6.5, 7, or 8. The study also evaluated LVV in 27.5 mM PIPES and 73 mM trehalose at pH 6.5 or 7. Based on >75% infectious titer recovery in transduced HOS cells, the least change was observed in the HEPES (pH 7), HEPES (pH 8), and PIPES (pH 7) formulations after 25° C. storage for 24 hours and 2-8° C. storage for 48 hours (FIG. 6). By hydrodynamic size, the HEPES (pH 8) formulation had an observed change after 25° C. storage for 24 hours and 2-8° C. storage for 48 hours. All formulations had a change in size after storage at 37° C. for 48 hours except for HEPES (pH 7) formulation (FIG. 7). By thermal unfolding measurements, the HEPES (pH 6.5) and PIPES (pH 6.5) formulations had an earlier onset of unfolding at approximately 40° C., compared to the PIPES (pH 7), HEPES (pH 7), and HEPES (pH 8) formulations which had onset of unfolding between 45-48° C. (FIG. 8). The PIPES (pH 7) and HEPES (pH 8) formulations appear to have two unfolding transitions, whereas the remaining formulations do not. Based on the first unfolding transition, PIPES (pH 6.5) and HEPES (pH 6.5) formulations have the highest unfolding temperature of approximately 60° C., followed by HEPES (pH 7) formulation which had an unfolding temperature of 54° C. Based on the overall assessment, LVV HEPES (pH 7) performed the best meeting many of the screening criteria.

### Summary of Experimental Results for Disaccharide and Salt Screening

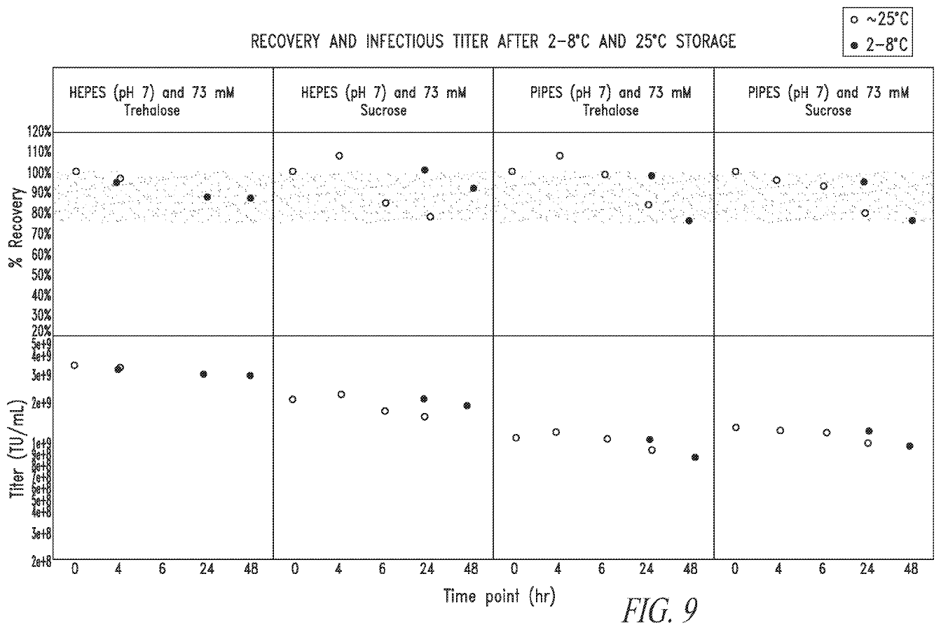

The carbohydrates trehalose and sucrose were compared in HEPES and PIPES buffers. Based on LVV stability (FIG. 9), no difference was observed therefore only sucrose combinations were also evaluated further.

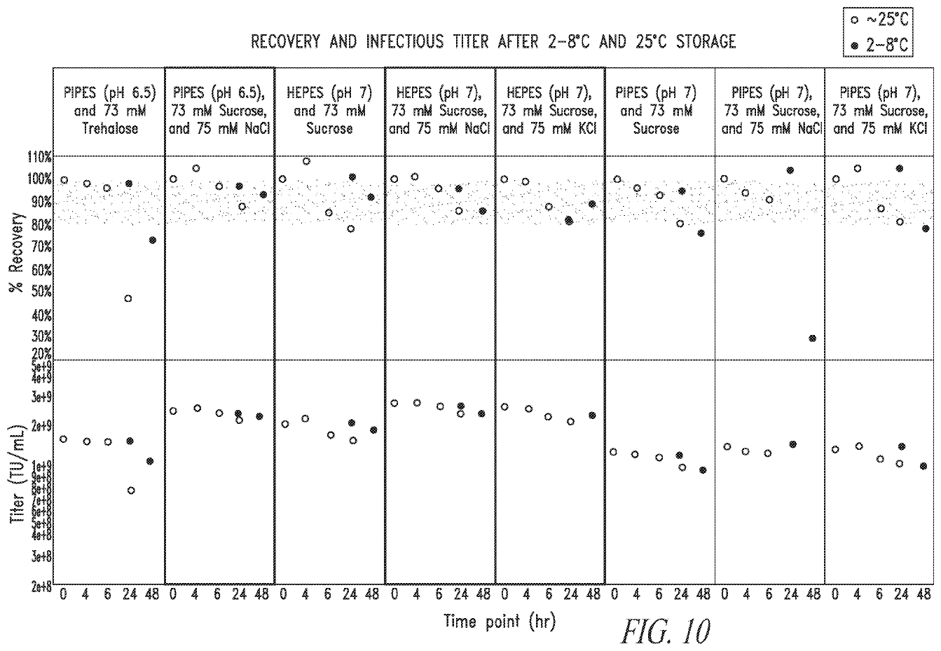

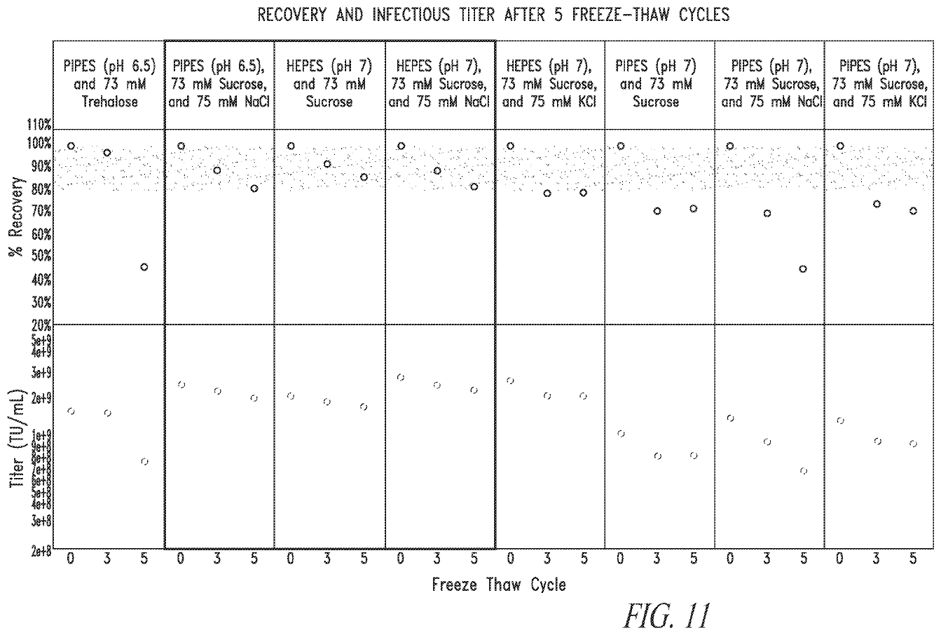

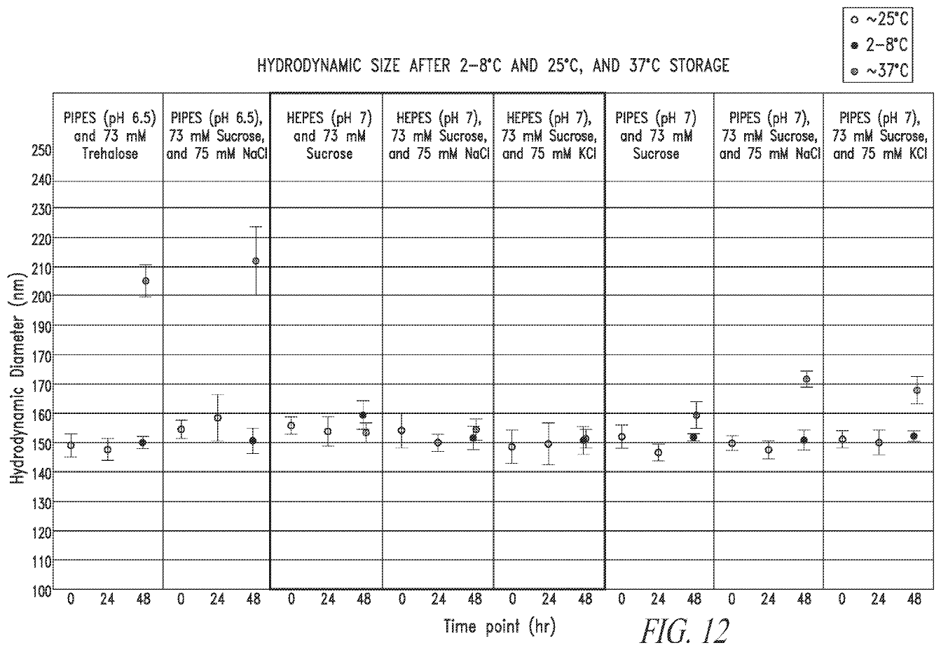

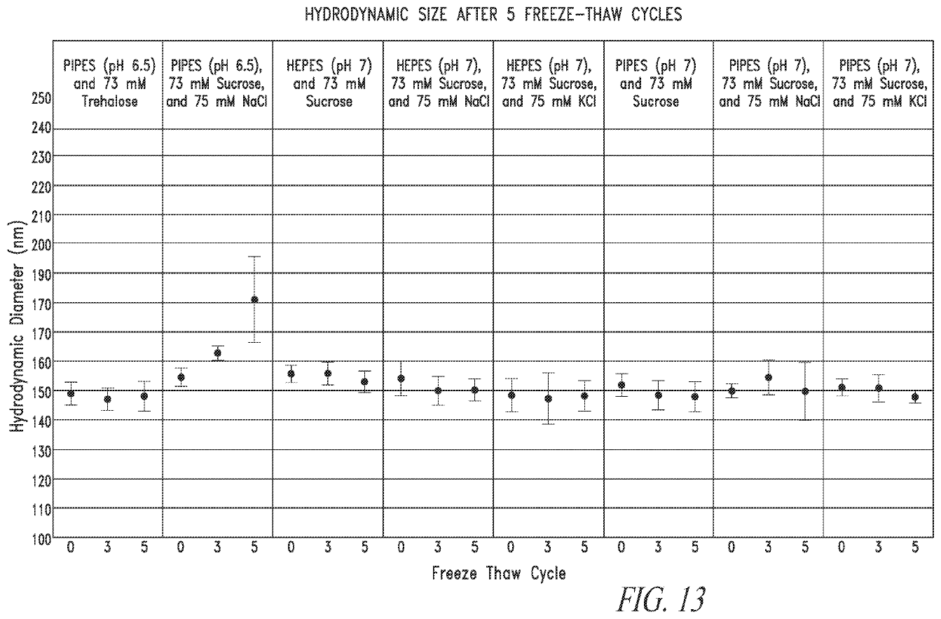

27.5 mM HEPES (pH 7) formulations with 73 mM sucrose and 73 mM sucrose in combination with 75 mM NaCl or KCl were compared. By infectious titer results, both HEPES-based and salt formulations were comparable after 25° C. storage for 24 hours and 2-8° C. storage for 48 hours (FIG. 10). By freeze-thaw stress (5 cycles), formulations with 73 mM sucrose only or with 75 mM NaCl had the least change by infectious titer measurements (FIG. 11). By hydrodynamic size, no HEPES based formulations had an observed change after 25° C. storage for 24 hours, 2-8° C. storage for 48 hours, and 37° C. for 48 hours (FIG. 12). No change in size was observed for the HEPES based formulations after five freeze thaw cycles (FIG. 13). From this study, 27.5 mM HEPES (pH 7) formulations containing 73 mM sucrose with and without 75 mM NaCl were the top formulation choices.

PIPES-based formulations were further evaluated as it has been reported in literature to be effective for lentiviruses. 27.5 mM PIPES (pH 6.5) with 73 mM trehalose, and 27.5 mM PIPES (pH 6.5) with 73 mM sucrose and 75 mM NaCl were compared. Additionally, 27.5 mM PIPES (pH 7) with 73 mM sucrose or 73 mM sucrose in combination with 75 mM NaCl or 75 mM KCl were evaluated. Based on >80% infectious titer recovery in transduced HOS cells, the PIPES (pH 6.5) formulation with 73 mM sucrose and 75 mM NaCl had the least change after 25° C. storage for 24 hours and 2-8° C. storage for 48 hours (FIG. 10). After freeze-thaw (5 cycles), the PIPES (pH 6.5) formulation with 73 mM sucrose and 75 mM NaCl had the highest infectious titer recovery (FIG. 11). By hydrodynamic size, no PIPES based formulation had an observed change after 25° C. storage for 24 hours and 2-8° C. storage for 48 hours. A change in size was observed for all PIPES based formulations after storage at 37° C. storage for 48 hours (FIG. 12). After five freeze thaw cycles, only the PIPES (pH 6.5) formulation with 73 mM sucrose and 75 mM NaCl had an increase in size (FIG. 13).

Based on the comparison of PIPES and HEPES based formulations at similar pH, LVV was surprisingly more stable in the HEPES based buffers, particularly by assessment of particle size under various conditions, and titer after freeze-thaw.

### Summary of Experimental Results for Additional Component Screening

A screening study was completed with several additional components to the buffering agent and sucrose to see if the additional components improved LVV stability with longer temperature hold durations at 168 hours at 5° C. and by monitoring of the number visible particles.

The formulations were comprised of 27.5 mM HEPES (pH 7) and:
73 mM sucrose and 75 mM NaCl
73 mM sucrose and 75 mM NaCl and 0.1 mg/mL P188
73 mM sucrose and 50 mM L-Pro
146 mM sucrose
146 mM sucrose and 75 mM NaCl
146 mM sucrose and 50 mM L-Arg
146 mM sucrose and 50 mM L-Arg and 50 mM L-Glu
146 mM sucrose and 0.1 mg/mL P188
146 mM sucrose and 16 mg/mL Inulin

The following formulations were also evaluated with 27.5 mM L-His (pH 7):
146 mM sucrose
146 mM sucrose and 50 mM L-Pro
146 mM sucrose and 16 mg/mL Inulin
275 mM sorbitol
73 mM sucrose and 75 mM NaCl
73 mM sucrose and 50 mM L-Pro
73 mM sucrose and 75 mM NaCl and 0.1 mg/mL P188

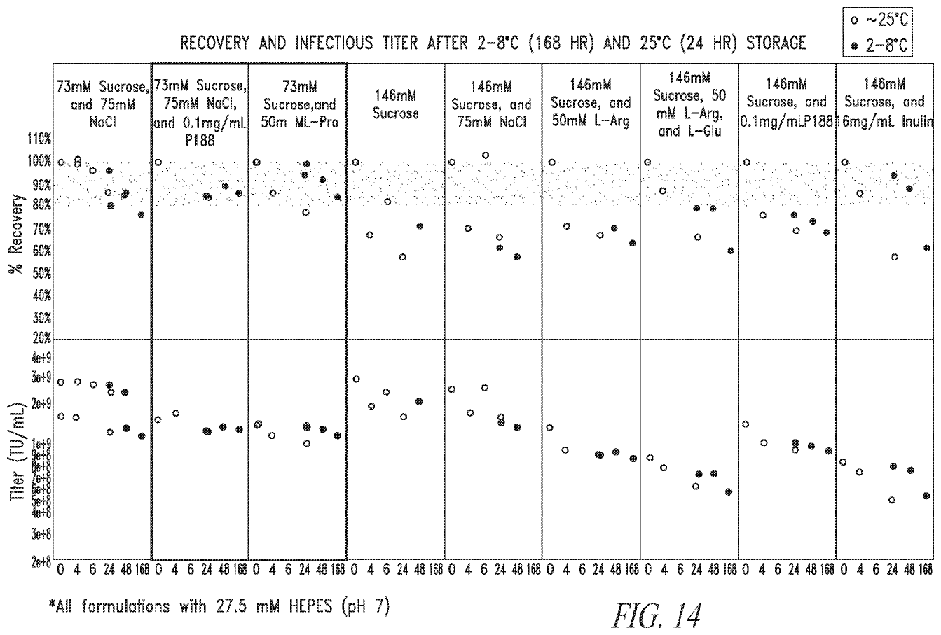

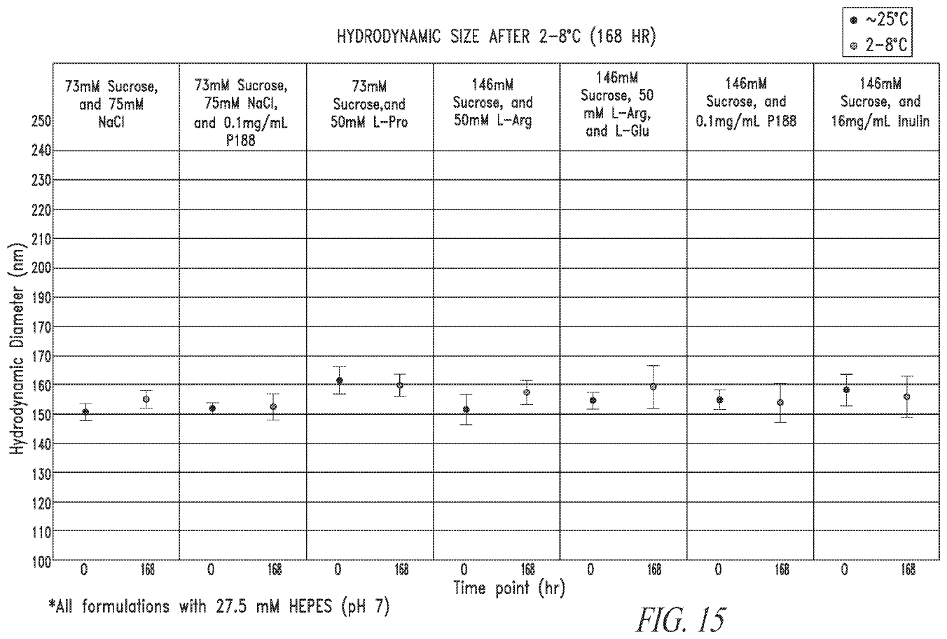

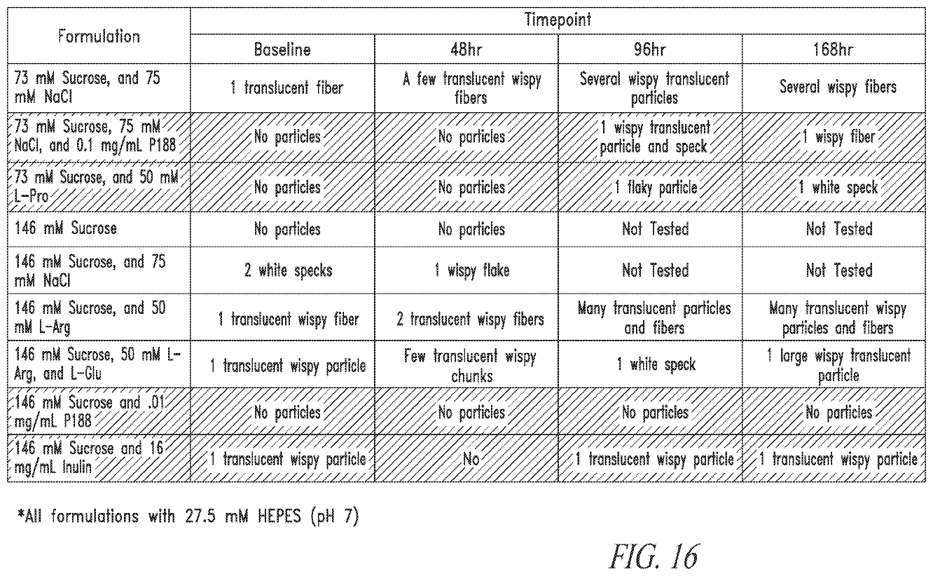

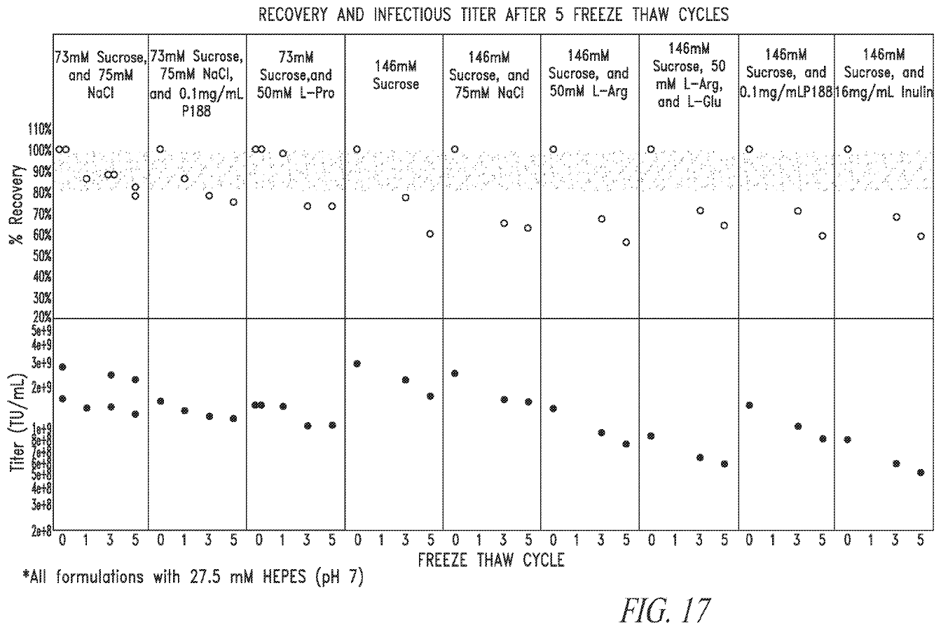

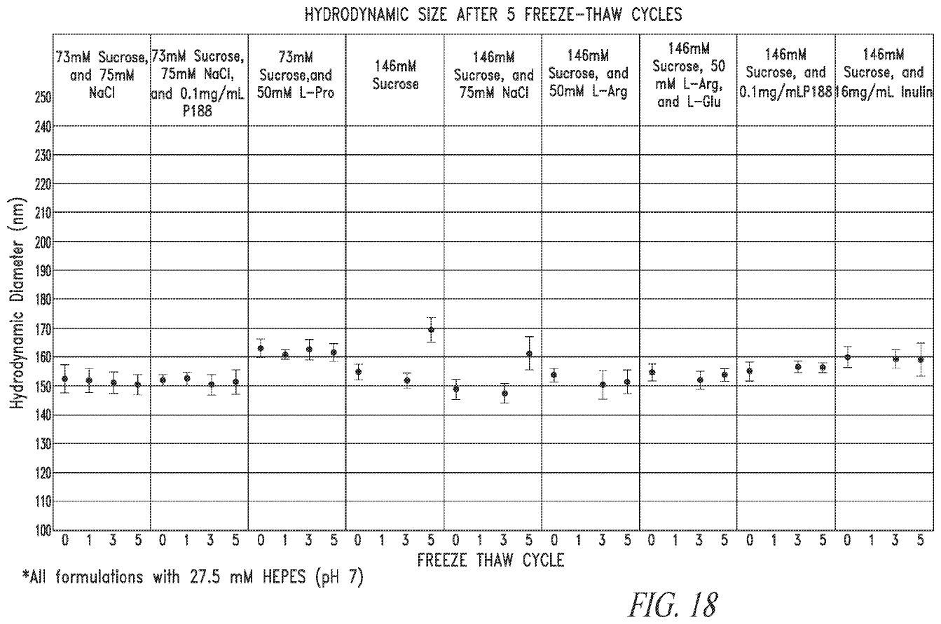

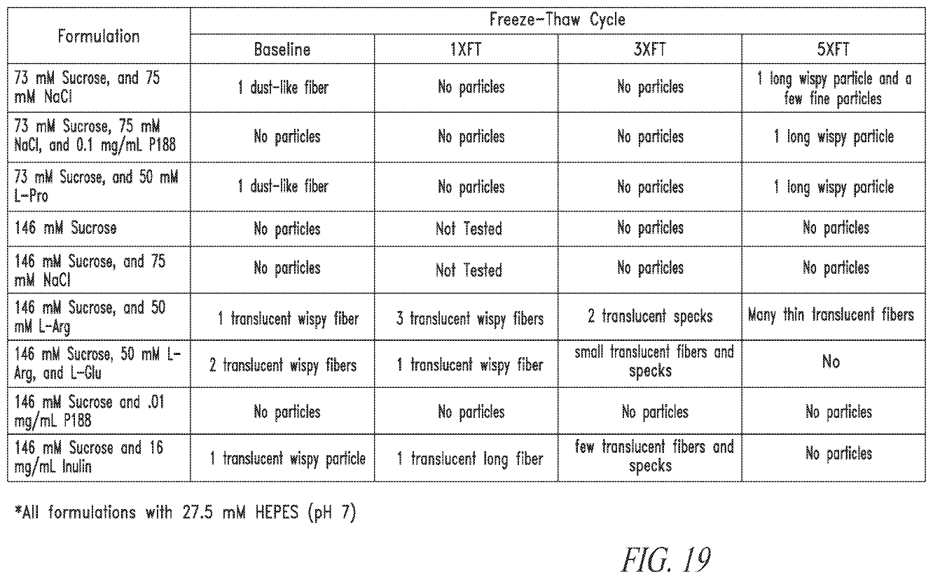

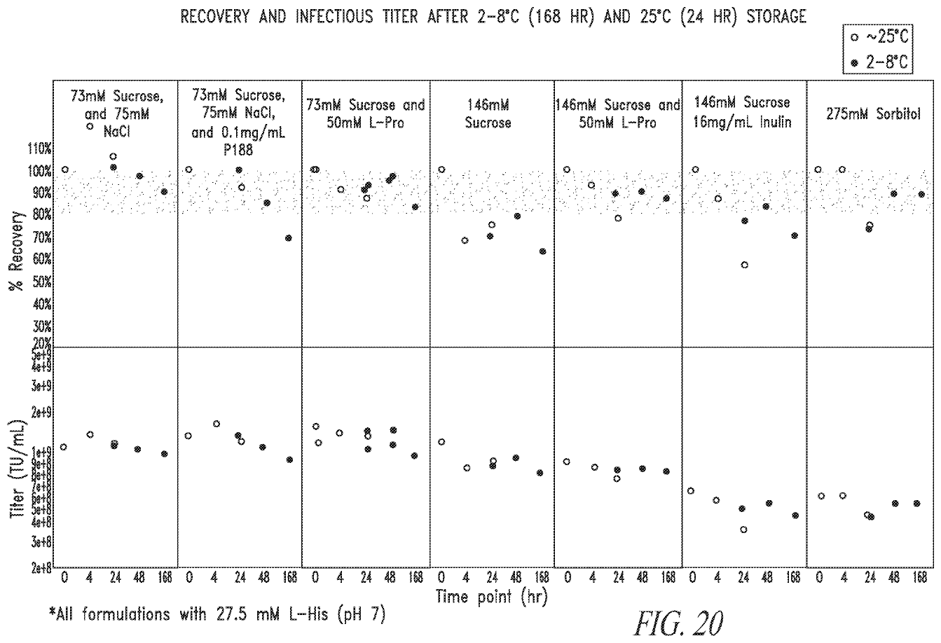

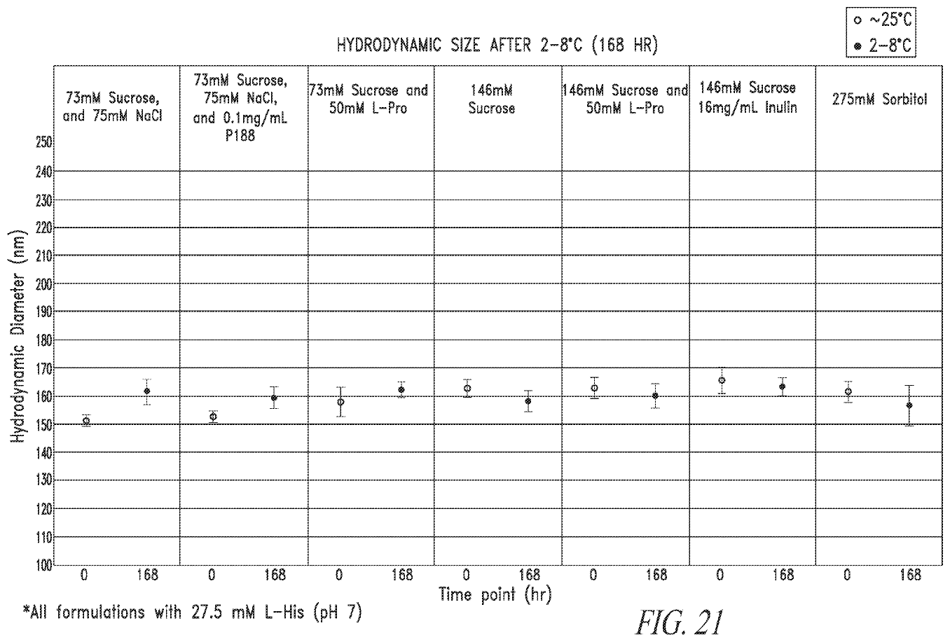

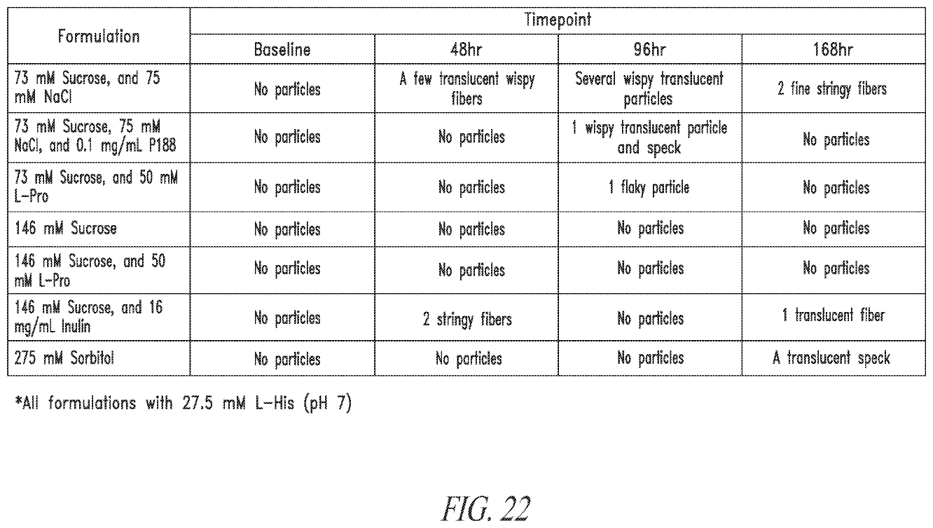

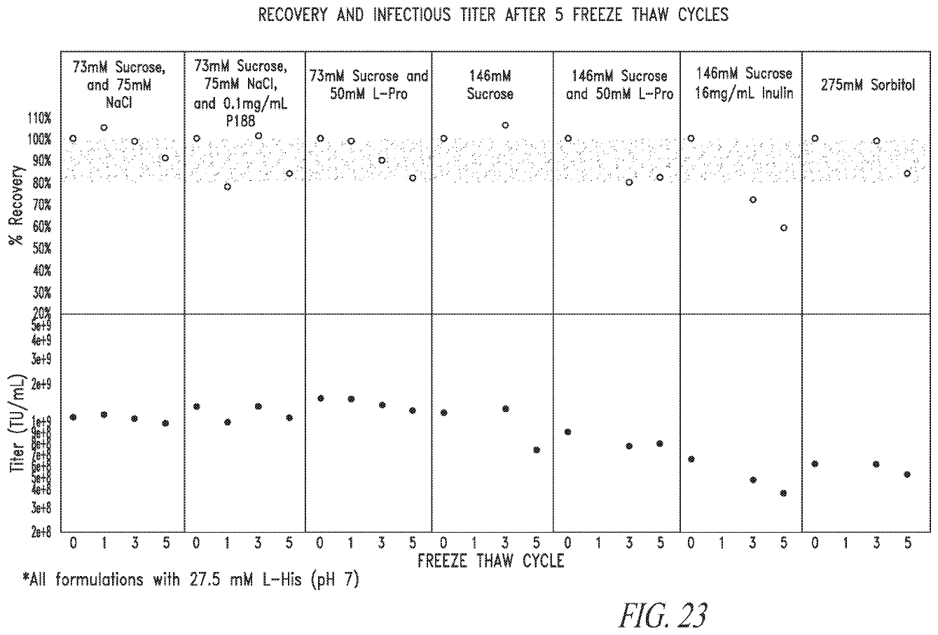

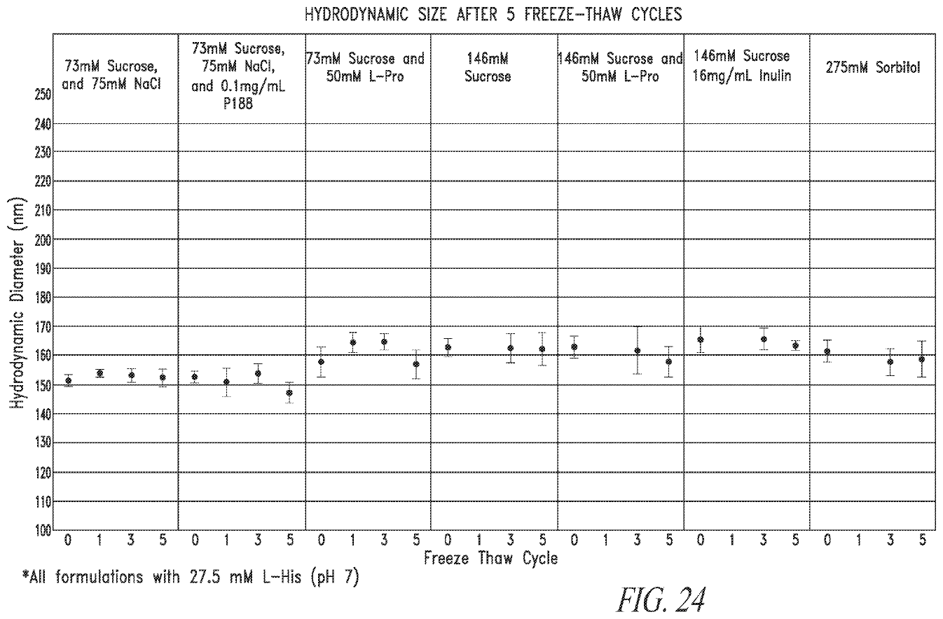

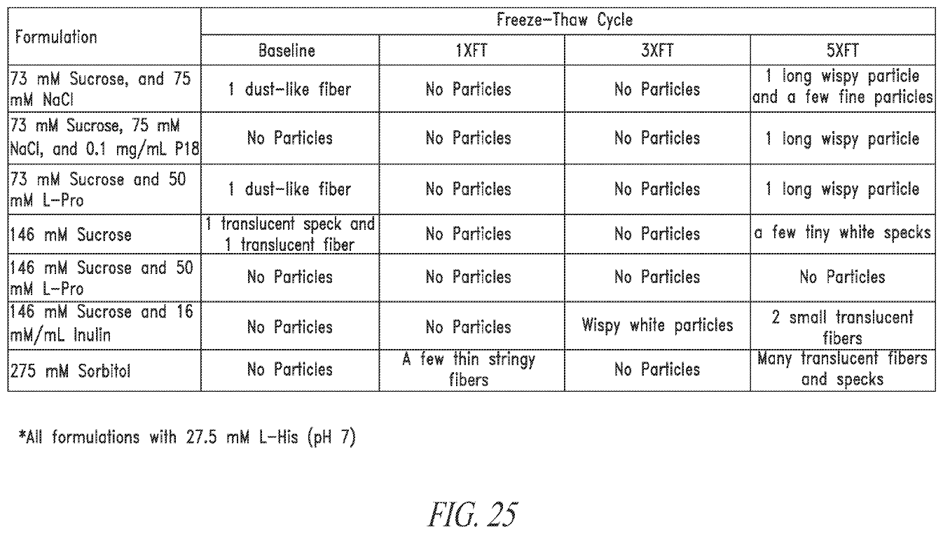

By infectious titer measurements, all 27.5 mM HEPES formulations (pH 7) consisting of 146 mM sucrose performed poorly after 25° C. storage for 24 hours and 2-8° C. storage for 168 hours (FIG. 14). 27.5 mM HEPES (pH 7) and 73 mM sucrose formulations with 75 mM NaCl, 75 mM NaCl and 0.1 mg/mL P188, or 50 mM L-Proline had the highest titer recovery at the different storage temperature (FIG. 14). All formulation had no change in hydrodynamic size after 2-8° C. storage for 168 hours (FIG. 15). Lastly, comparing only the 73 mM sucrose-based formulations, the fewest number of visible particles were observed for the formulations containing 0.1 mg/mL Poloxamer188 and 50 mM L-Proline after storage at 2-8° C. temperature. Similarly, the formulations 27.5 mM HEPES (pH 7) formulation with 73 mM sucrose, 75 mM NaCl, and 0.1 mg/mL poloxamer188, or 73 mM sucrose and 50 mM L-Proline had a high titer recovery, no change in hydrodynamic size, and few number of visible particles after freezing at ≤−65° C. for greater than 1.5 hours and thawing at 30° C. for 1.5 hours. (FIG. 16-19). In a similar evaluation, the 27.5 mM L-histidine (pH 7) formulation with 73 mM sucrose and 50 mM L-Pro showed the highest infectious titer recovery, no change in hydrodynamic size, and least number visible particles after 25° C. storage for 24 hours, after 2-8° C. storage for 168 hours, and after one freeze-thaw cycle (FIGS. 20-25).

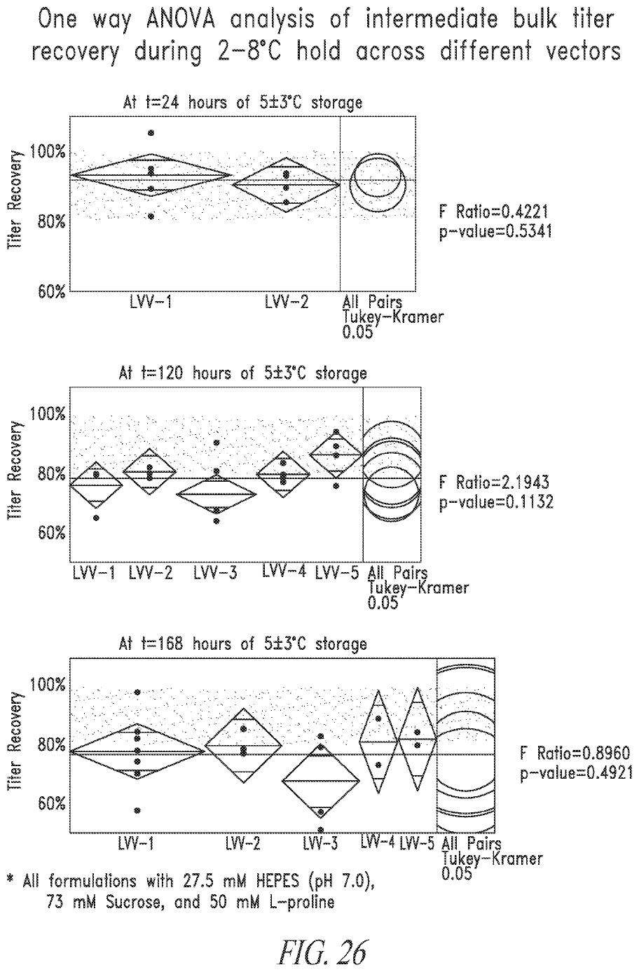

The 27.5 mM HEPES (pH 7), 73 mM Sucrose, and 50 mM L-Pro formulation was evaluated across multiple lentiviral vectors (e.g., LVV1, LVV2, LVV3, LVV4, and LVV5) that encode for various constructs targeting different genes of interest. The titer recovery after 2-8° C. storage for up to 168 hours was evaluated by statistical analysis (FIG. 26). The analysis was grouped by time at 24, 120 and 168 hours of hold. One way ANOVA followed by Tukey Kramer analysis was performed. The p-value comparing the mean titer recovery was insignificant (greater than 0.05) and indicated that the formulation performance was similar for each LVV.

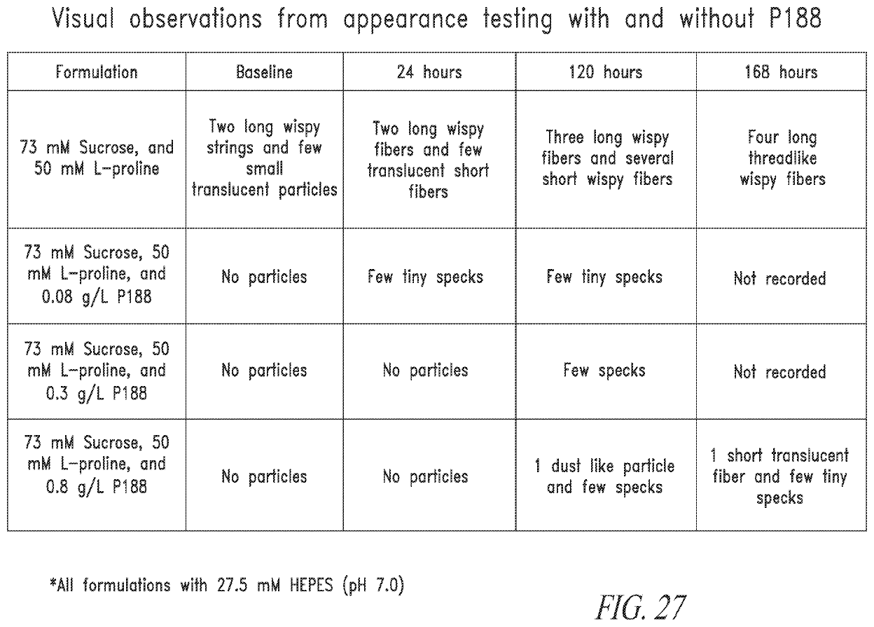

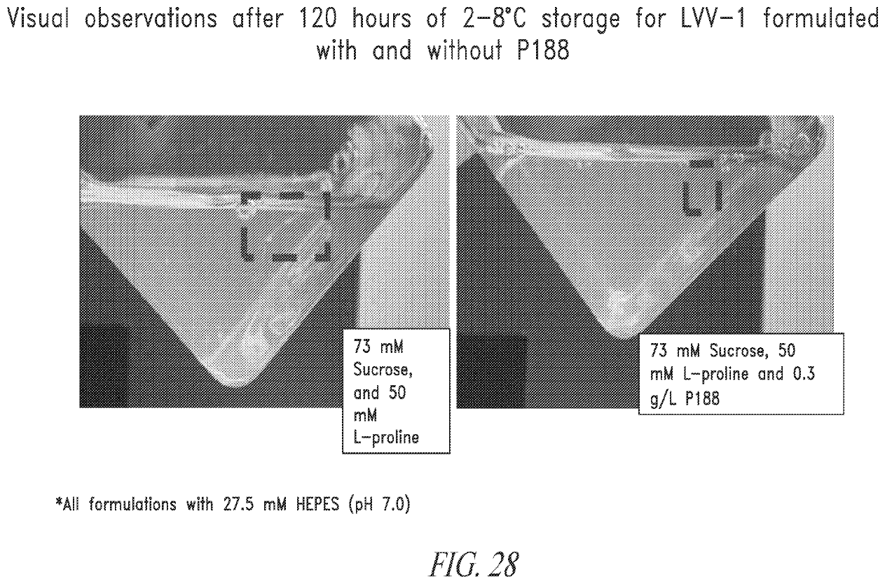

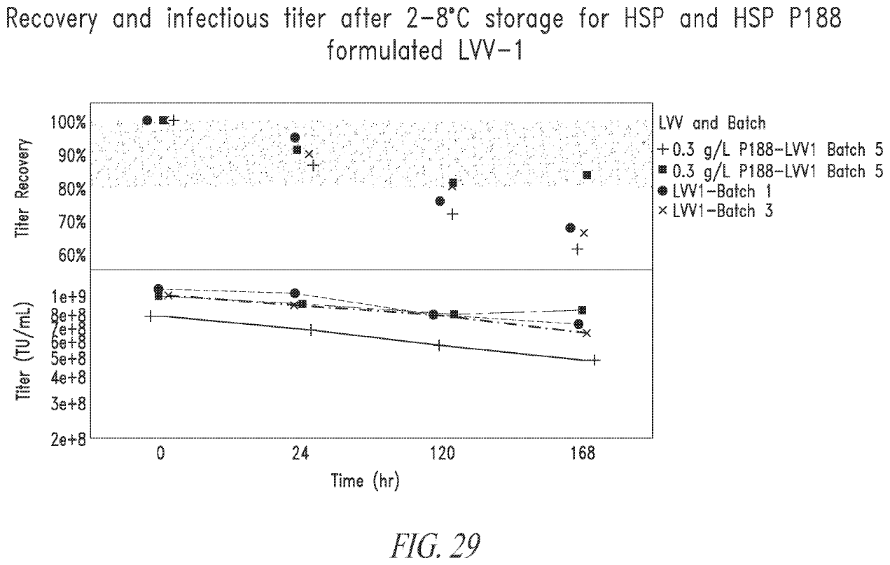

Additional evaluation with poloxamer 188 was performed to reduce the number of visible particles sometimes observed after manufacturing. Formulations with 27.5 mM HEPES (pH 7), 73 mM Sucrose, and 50 mM L-Pro only and with the addition of 0.08 g/L, 0.3 g/L, or 0.8 g/L Poloxamer 188 were visually compared after storage at 2-8° C. storage (FIGS. 27 and 28). Addition of at least 0.3 g/L reduced the appearance of particulates after 24 hours and minimized the type of particles after 120 hours storage compared to formulations no P188 and 0.08% P188. The infectious titer of LVV formulated with 27.5 mM HEPES (pH 7), 73 mM Sucrose, 50 mM L-Pro, and 0.3 g/L P188 was monitored for 168 hours at 2-8° C. and compared to formulation without P188 (FIG. 29). Both formulations had similar performance indicating that P188 addition does not impact infectious titer recovery.

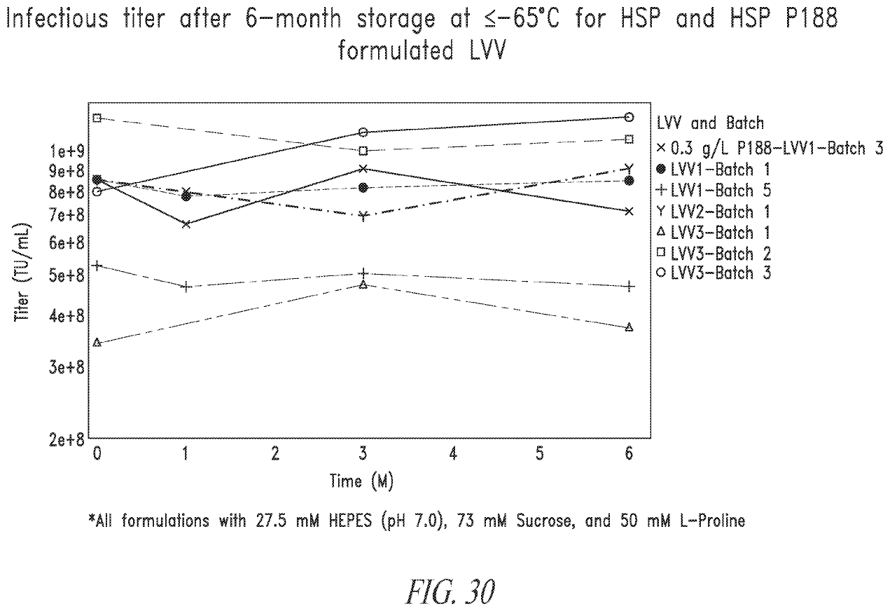

Lastly it is of interest to store LVV at ≤−65° C. for long durations. Formulations with and without P188 were compared for up to 6 months at ≤−65° C. for LVV1, LVV2, and LVV3. All LVV and formulations maintained infectious titer levels for up to 6 months storage (FIG. 30).

### Example 3

### Analysis of Lentiviral Potency

The potency of the lentivirus was measured in peripheral blood mononuclear cells (PBMCs). PBMCs are transduced with the lentivirus at various multiplicity of infection (MOIs of 2.5, 5, 10, and 20) and measured for the relative frequency of chimeric antigen receptor expression (% CAR+ cells). CAR+ is detected by a fluorescently tagged protein that binds against the CAR and cell populations are evaluated by flow cytometry. Relative potency is reported as a relative measurement against a reference standard.

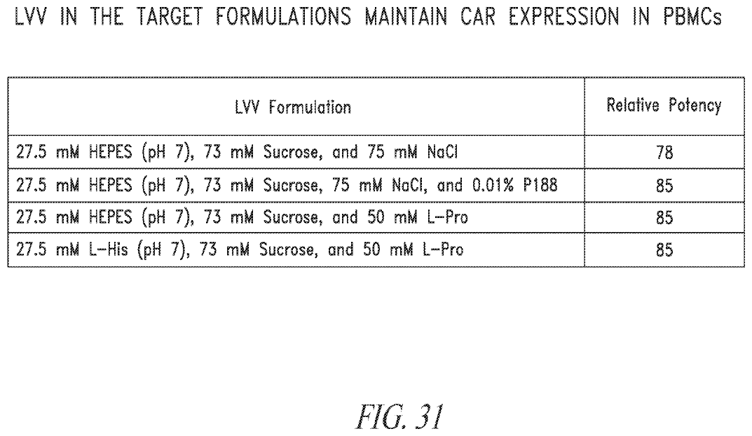

The following formulations had the highest infectious titer recovery after storage at 25° C., 2-8° C., and multiple freeze thaw cycles which are relevant to the lentivirus manufacturing process. As a confirmation of compatibility with the drug product process, relative potency measurements were performed and confirmed to have no significant impact to potency (expression of CAR+) and stabilized LVV (FIG. 31).
27.5 mM HEPES (pH 7), 73 mM Sucrose, and 75 mM NaCl
27.5 mM HEPES (pH 7), 73 mM Sucrose, 75 mM NaCl, and 0.1 mg/mL P188
27.5 mM HEPES (pH 7), 73 mM Sucrose, and 50 mM L-Pro
27.5 mM L-His (pH 7), 73 mM Sucrose, and 50 mM L-Pro

The top formulations evaluated here met criteria required for manufacturing processes. Extended duration at 25° C. and 2-8° C. allows for flexibility in many manufacturing operations and the stability appeared to be maintained with HEPES (pH 7) and L-His (pH 7) and sucrose combinations. Additionally, the freeze-thaw process significantly reduces the recovery of the lentiviral vector. The studies show that the addition of sodium chloride or L-proline in some instances improve recovery after a single freeze-thaw and at times up to five freeze thaw cycles. Lastly, in the fill-finish process, reduction of visible particles may indicate aggregation of particles within the solution. The poloxamer 188 and L-proline appeared to have minimized these interactions.

### Embodiments

The present disclosure contemplates the following embodiments:
1. An aqueous viral composition comprising:
   a) a viral vector
   b) a HEPES or L-Histidine buffer;
   c) a carbohydrate; and
   d) an amino acid.
2. The composition of embodiment 1, wherein the buffer is present at a concentration of about 25 mM to about 30 mM.
3. The composition of any one of the preceding embodiments, wherein the buffer is present at a concentration of about 26 mM to about 29 mM.
4. The composition of any one of the preceding embodiments, wherein the buffer is present at a concentration of about 27 mM to about 28 mM.
5. The composition of any one of the preceding embodiments, wherein the buffer is present at a concentration of about 27.5 mM.
6. The composition of any one of embodiments 1-5, wherein the buffer is a HEPES buffer.
7. The composition of any one of embodiments 1-5, wherein the buffer is an L-Histidine buffer.
8. The composition of any one of the preceding embodiments, wherein the carbohydrate is present at a concentration of about 66 mM to about 80 mM.
9 The composition of any one of the preceding embodiments, wherein the carbohydrate is present at a concentration of about 67 mM to about 79 mM.
10. The composition of any one of the preceding embodiments, wherein the carbohydrate is present at a concentration of about 68 mM to about 78 mM.
11. The composition of any one of the preceding embodiments, wherein the carbohydrate is present at a concentration of about 69 mM to about 77 mM.
12. The composition of any one of the preceding embodiments, wherein the carbohydrate is present at a concentration of about 70 mM to about 76 mM.
13. The composition of any one of the preceding embodiments, wherein the carbohydrate is present at a concentration of about 71 mM to about 75 mM.
14. The composition of any one of the preceding embodiments, wherein the carbohydrate is present at a concentration of about 72 mM to about 74 mM.
15. The composition of any one of the preceding embodiments, wherein the carbohydrate is present at a concentration of about 73 mM.
16. The composition of any one of embodiments 1-12, wherein the carbohydrate is present at a concentration of about 2.0% to about 3.0% by weight per volume of the composition.
17. The composition of any one of embodiments 1-12, wherein the carbohydrate is present at a concentration of about 2.1% to about 2.9% by weight per volume of the composition.
18. The composition of any one of embodiments 1-12, wherein the carbohydrate is present at a concentration of about 2.2% to about 2.8% by weight per volume of the composition.
19. The composition of any one of embodiments 1-12, wherein the carbohydrate is present at a concentration of about 2.3% to about 2.7% by weight per volume of the composition.
20. The composition of any one of embodiments 1-12, wherein the carbohydrate is present at a concentration of about 2.4% to about 2.6% by weight per volume of the composition.
21. The composition of any one of embodiments 1-12, wherein the carbohydrate is present at a concentration of about 2.5% by weight per volume of the composition.
22. The composition of any one of embodiments 1-21, wherein the carbohydrate is a disaccharide.
23. The composition of any one of embodiments 1-21, wherein the carbohydrate is lactose, glucose, mannose, mannitol, sorbitol, sucrose, trehalose, and/or glycerol
24. The composition of any one of embodiments 1-21, wherein the carbohydrate is sucrose and/or trehalose.
25. The composition of any one of embodiments 1-24, wherein the carbohydrate is sucrose.
26. The composition of any one of embodiment 1-24, wherein the carbohydrate is trehalose
27. The composition of any one of embodiments 1-26, wherein the amino acid is present at a concentration of about 40 mM to about 60 mM.
28. The composition of any one of embodiments 1-26, wherein the amino acid is present at a concentration of about 41 mM to about 59 mM.
29. The composition of any one of embodiments 1-26, wherein the amino acid is present at a concentration of about 42 mM to about 58 mM.
30. The composition of any one of embodiments 1-26, wherein the amino acid is present at a concentration of about 43 mM to about 57 mM.
31. The composition of any one of embodiments 1-26, wherein the amino acid is present at a concentration of about 44 mM to about 56 mM.
32. The composition of any one of embodiments 1-26, wherein the amino acid is present at a concentration of about 45 mM to about 55 mM.
33. The composition of any one of embodiments 1-26, wherein the amino acid is present at a concentration of about 46 mM to about 54 mM.
34. The composition of any one of embodiments 1-26, wherein the amino acid is present at a concentration of about 47 mM to about 53 mM.
35. The composition of any one of embodiments 1-26, wherein the amino acid is present at a concentration of about 48 mM to about 52 mM.
36. The composition of any one of embodiments 1-26, wherein the amino acid is present at a concentration of about 49 mM to about 51 mM.
37. The composition of any one of embodiments 1-26, wherein the amino acid is present at a concentration of about 50 mM.
38. The composition of any one of embodiments 1-37, wherein the amino acid is a non-polar amino acid.
39. The composition of any one of embodiments 1-37, wherein the amino acid is selected from the group consisting of: glycine, alanine, valine, leucine, methionine, isoleucine phenylalanine, tyrosine, and tryptophan.
40. The composition of any one of embodiments 1-37, wherein the amino acid is selected from the group consisting of: phenylalanine, tyrosine, tryptophan, and proline.
41. The composition of any one of embodiments 1-37, wherein the amino acid is L-Proline.
42. The composition of any one of the preceding embodiments, wherein the composition further comprises a salt.
43. The composition of embodiment 42, wherein the salt is present at a concentration of about 65 mM to about 85 mM.
44. The composition of embodiment 42, wherein the salt is present at a concentration of about 66 mM to about 84 mM.
45. The composition of embodiment 42, wherein the salt is present at a concentration of about 67 mM to about 83 mM.
46. The composition of embodiment 42, wherein the salt is present at a concentration of about 68 mM to about 82 mM.
47. The composition of embodiment 42, wherein the salt is present at a concentration of about 69 mM to about 81 mM.
48. The composition of embodiment 42, wherein the salt is present at a concentration of about 70 mM to about 80 mM.
49. The composition of embodiment 42, wherein the salt is present at a concentration of about 71 mM to about 79 mM.
50. The composition of embodiment 42, wherein the salt is present at a concentration of about 72 mM to about 78 mM.
51. The composition of embodiment 42, wherein the salt is present at a concentration of about 73 mM to about 77 mM.
52. The composition of embodiment 42, wherein the salt is present at a concentration of about 74 mM to about 76 mM.
53. The composition of embodiment 42, wherein the salt is present at a concentration of about 75 mM.
54. The composition of any one of embodiments 42-53, wherein the salt is a chloride salt, KCl, or NaCl.
55. The composition of any one of embodiments 42-54, wherein the salt is NaCl.
56. The composition of any one of the preceding embodiments, wherein the composition further comprises a poloxamer.
57. The composition of embodiment 56, wherein the poloxamer is present at a concentration of about 0.01 mg/ml to about 1 mg/ml.
58. The composition of embodiment 56, wherein the poloxamer is present at a concentration of about 0.02 mg/ml to about 0.9 mg/ml.
59. The composition of embodiment 56, wherein the poloxamer is present at a concentration of about 0.03 mg/ml to about 0.8 mg/ml.
60. The composition of embodiment 56, wherein the poloxamer is present at a concentration of about 0.04 mg/ml to about 0.7 mg/ml.
61. The composition of embodiment 56, wherein the poloxamer is present at a concentration of about 0.05 mg/ml to about 0.6 mg/ml.
62. The composition of embodiment 56, wherein the poloxamer is present at a concentration of about 0.06 mg/ml to about 0.5 mg/ml.
63. The composition of embodiment 56, wherein the poloxamer is present at a concentration of about 0.07 mg/ml to about 0.4 mg/ml.
64. The composition of embodiment 56, wherein the poloxamer is present at a concentration of about 0.08 mg/ml to about 0.3 mg/ml.
65. The composition of embodiment 56, wherein the poloxamer is present at a concentration of about 0.09 mg/ml to about 0.2 mg/ml.
66. The composition of embodiment 56, wherein the poloxamer is present at a concentration of about 0.1 mg/ml.
67. The composition of embodiment 56, wherein the poloxamer is present at a concentration of about 0.1 mg/ml to about 0.5 mg/ml.
68. The composition of embodiment 56, wherein the poloxamer is present at a concentration of about 0.2 mg/ml to about 0.4 mg/ml.
69. The composition of embodiment 56, wherein the poloxamer is present at a concentration of about 0.3 mg/ml.
70. The composition of any one of embodiments 56-69, wherein the poloxamer is poloxamer 188, poloxamer 288, poloxamer 335, poloxamer 338, and poloxamer 407.
71. The composition of any one of embodiments 56-70, wherein the poloxamer is poloxamer 188.
72. The composition of any one of the preceding embodiments, wherein the composition comprises a pH of about 6.5 to about 8.
73. The composition of any one of the preceding embodiments, wherein the composition comprises a pH of about 6.5.
74. The composition of any one of the preceding embodiments, wherein the composition comprises a pH of about 7.
75. The composition of any one of the preceding embodiments, wherein the composition comprises a pH of about 7.5.
76. The composition of any one of the preceding embodiments, wherein the composition comprises a pH of about 8.
77. An aqueous viral composition comprising:
   a) a viral vector,
   b) about 27.5 mM HEPES,
   c) about 73 mM sucrose or about 2.5% sucrose by weight per volume of composition, and
   d) about 50 mM L-Proline; wherein the composition comprises a pH of about 7.
78. An aqueous viral composition comprising:
   a) a viral vector,
   b) about 27.5 mM L-Histidine,
   c) about 73 mM sucrose or about 2.5% sucrose by weight per volume of composition, and
   d) about 50 mM L-Proline;
   wherein the composition comprises a pH of about 7.
79. An aqueous viral composition comprising:
   a) a viral vector,
   b) about 27.5 mM HEPES,
   c) about 73 mM sucrose or about 2.5% sucrose by weight per volume of composition,
   d) about 50 mM L-Proline, and
   e) about 0.1 mg/mL to about 0.8 mg/ml poloxamer 188;
   wherein the composition comprises a pH of about 7.
80. An aqueous viral composition comprising:
   a) a viral vector,
   b) about 27.5 mM HEPES,
   c) about 73 mM sucrose or about 2.5% sucrose by weight per volume of composition,
   d) about 50 mM L-Proline, and
   e) about 0.3 mg/mL poloxamer 188;
   wherein the composition comprises a pH of about 7.
81. An aqueous viral composition comprising:
   a) a viral vector,
   b) about 27.5 mM HEPES,
   c) about 73 mM sucrose or about 2.5% sucrose by weight per volume of composition,
   d) about 50 mM L-Proline, and
   e) about 75 mM NaCl;
   wherein the composition comprises a pH of about 7.
82. An aqueous viral composition comprising:
   a) a viral vector,
   b) about 27.5 mM HEPES,
   c) about 73 mM sucrose or about 2.5% sucrose by weight per volume of composition,
   d) about 50 mM L-Proline,
   e) about 75 mM NaCl, and
   f) about 0.1 to about 0.8 mg/mL poloxamer 188;
   wherein the composition comprises a pH of about 7.
83. An aqueous viral composition comprising:
   a) a viral vector,
   b) a HEPES buffer,
   c) a carbohydrate,
   d) a salt; and
   e) a poloxamer.
84. The composition of embodiment 83, wherein the composition does not comprise L-Proline.
85. The composition of embodiment 83 or embodiment 84, wherein the HEPES buffer is present at a concentration of about 25 mM to about 30 mM.
86. The composition of embodiment 83 or embodiment 84, wherein the HEPES buffer is present at a concentration of about 26 mM to about 29 mM.
87. The composition of embodiment 83 or embodiment 84, wherein the HEPES buffer is present at a concentration of about 27 mM to about 28 mM.
88. The composition of embodiment 83 or embodiment 84, wherein the HEPES buffer is present at a concentration of about 27.5 mM.
89. The composition of any one of embodiments 83-88, wherein the carbohydrate is present at a concentration of about 66 mM to about 80 mM.
90. The composition of any one of embodiments 83-88, wherein the carbohydrate is present at a concentration of about 67 mM to about 79 mM.
91. The composition of any one of embodiments 83-88, wherein the carbohydrate is present at a concentration of about 68 mM to about 78 mM.
92. The composition of any one of embodiments 83-88, wherein the carbohydrate is present at a concentration of about 69 mM to about 77 mM.
93. The composition of any one of embodiments 83-88, wherein the carbohydrate is present at a concentration of about 70 mM to about 76 mM.
94. The composition of any one of embodiments 83-88, wherein the carbohydrate is present at a concentration of about 71 mM to about 75 mM.
95. The composition of any one of embodiments 83-88, wherein the carbohydrate is present at a concentration of about 72 mM to about 74 mM.
96. The composition of any one of embodiments 83-88, wherein the carbohydrate is present at a concentration of about 73 mM.
97. The composition of any one of embodiments 83-88, wherein the carbohydrate is present at a concentration of about 2.0% to about 3.0% by weight per volume of the composition.
98. The composition of any one of embodiments 83-88, wherein the carbohydrate is present at a concentration of about 2.1% to about 2.9% by weight per volume of the composition.
99. The composition of any one of embodiments 83-88, wherein the carbohydrate is present at a concentration of about 2.2% to about 2.8% by weight per volume of the composition.
100. The composition of any one of embodiments 83-88, wherein the carbohydrate is present at a concentration of about 2.3% to about 2.7% by weight per volume of the composition.
101. The composition of any one of embodiments 83-88, wherein the carbohydrate is present at a concentration of about 2.4% to about 2.6% by weight per volume of the composition. 102. The composition of any one of embodiments 83-88, wherein the carbohydrate is present at a concentration of about 2.5% by weight per volume of the composition.
103. The composition of any one of embodiments 83-102, wherein the carbohydrate is a disaccharide.
104. The composition of any one of embodiments 83-102, wherein the carbohydrate is lactose, glucose, mannose, mannitol, sorbitol, sucrose, trehalose, and/or glycerol
105. The composition of any one of embodiments 83-102, wherein the carbohydrate is sucrose and/or trehalose.
106. The composition of any one of embodiments 83-102, wherein the carbohydrate is sucrose.
107. The composition of any one of embodiments 83-102, wherein the carbohydrate is trehalose.
108. The composition of any one of embodiments 83-107, wherein the salt is present at a concentration of about 65 mM to about 85 mM.
109. The composition of any one of embodiments 83-107, wherein the salt is present at a concentration of about 66 mM to about 84 mM.
110. The composition of any one of embodiments 83-107, wherein the salt is present at a concentration of about 67 mM to about 83 mM.
111. The composition of any one of embodiments 83-107, wherein the salt is present at a concentration of about 68 mM to about 82 mM.
112. The composition of any one of embodiments 83-107, wherein the salt is present at a concentration of about 69 mM to about 81 mM.
113. The composition of any one of embodiments 83-107, wherein the salt is present at a concentration of about 70 mM to about 80 mM.
114. The composition of any one of embodiments 83-107, wherein the salt is present at a concentration of about 71 mM to about 79 mM.
115. The composition of any one of embodiments 83-107, wherein the salt is present at a concentration of about 72 mM to about 78 mM.
116. The composition of any one of embodiments 83-107, wherein the salt is present at a concentration of about 73 mM to about 77 mM.
117. The composition of any one of embodiments 83-107, wherein the salt is present at a concentration of about 74 mM to about 76 mM.
118. The composition of any one of embodiments 83-107, wherein the salt is present at a concentration of about 75 mM.
119. The composition of any one of embodiments 83-118, wherein the salt is KCl or NaCl.
120. The composition of embodiment 119, wherein the salt is NaCl.
121. The composition of any one of embodiments 83-120, wherein the poloxamer is present at a concentration of about 0.01 mg/ml to about 1 mg/ml.
122. The composition of any one of embodiments 83-120, wherein the poloxamer is present at a concentration of about 0.02 mg/ml to about 0.9 mg/ml.
123. The composition of any one of embodiments 83-120, wherein the poloxamer is present at a concentration of about 0.03 mg/ml to about 0.8 mg/ml.
124. The composition of any one of embodiments 83-120, wherein the poloxamer is present at a concentration of about 0.04 mg/ml to about 0.7 mg/ml.
125. The composition of any one of embodiments 83-120, wherein the poloxamer is present at a concentration of about 0.05 mg/ml to about 0.6 mg/ml.
126. The composition of any one of embodiments 83-120, wherein the poloxamer is present at a concentration of about 0.06 mg/ml to about 0.5 mg/ml.
127. The composition of any one of embodiments 83-120, wherein the poloxamer is present at a concentration of about 0.07 mg/ml to about 0.4 mg/ml.
128. The composition of any one of embodiments 83-120, wherein the poloxamer is present at a concentration of about 0.08 mg/ml to about 0.3 mg/ml.
129. The composition of any one of embodiments 83-120, wherein the poloxamer is present at a concentration of about 0.09 mg/ml to about 0.2 mg/ml.
130. The composition of any one of embodiments 83-120, wherein the poloxamer is present at a concentration of about 0.1 mg/ml.
131. The composition of any one of embodiments 83-130, wherein the poloxamer is poloxamer 188, poloxamer 288, poloxamer 335, poloxamer 338, and poloxamer 407.
132. The composition of any one of embodiments 83-130, wherein the poloxamer is poloxamer 188 (P188).
133. The composition of any one of embodiments 83-132, wherein the composition comprises a pH of about 7 to about 8.
134. The composition of any one of embodiments 83-132, wherein the composition comprises a pH of about 7.
135. The composition of any one of embodiments 83-132, wherein the composition comprises a pH of about 7.5.
136. The composition of any one of embodiments 83-132, wherein the composition comprises a pH of about 8.
137. An aqueous viral composition comprising:
   a) a viral vector,
   b) about 27.5 mM HEPES,
   c) about 73 mM sucrose by weight per volume of composition,
   d) about 75 mM NaCl, and
   e) about 0.1 to about 0.8 mg/ml poloxamer 188;
   wherein the composition comprises a pH of about 7.
138. The composition of any one of the preceding embodiments, wherein the viral vector is present at a titer from about 1×10⁸ to about 2×10⁹ TU/ml.
139. The composition of any one of the preceding embodiments, wherein the viral vector is present at a titer of about 1×10⁸ TU/ml.
140. The composition of any one of the preceding embodiments, wherein the viral vector is present at a titer of about 2×10⁸ TU/ml.
141. The composition of any one of embodiments 1-139, wherein the viral vector is present at a titer of about 3×10⁸ TU/ml.
142. The composition of any one of embodiments 1-139, wherein the viral vector is present at a titer of about 4×10⁸ TU/ml.
143. The composition of any one of embodiments 1-139, wherein the viral vector is present at a titer of about 5×10⁸ TU/ml.
144. The composition of any one of embodiments 1-139, wherein the viral vector is present at a titer of about 6×10⁸ TU/ml.
145. The composition of any one of embodiments 1-139, wherein the viral vector is present at a titer of about 7×10⁸ TU/ml.
146. The composition of any one of embodiments 1-139, wherein the viral vector is present at a titer of about 8×10⁸ TU/ml.
147. The composition of any one of embodiments 1-139, wherein the viral vector is present at a titer of about 9×10⁸ TU/ml.
148. The composition of any one of embodiments 1-139, wherein the viral vector is present at a titer of about 1×10⁹ TU/ml.
149. The composition of any one of embodiments 1-139, wherein the viral vector is present at a titer of about 2×10⁹ TU/ml.
150. The composition of any one of the preceding embodiments, wherein the vector is an adenoviral vector, an adeno-associated viral (AAV) vector, a herpes virus vector, a vaccinia virus vector, or a retroviral vector.
151. The composition of any one of the preceding embodiments, wherein the viral vector is a lentiviral vector.
152. The composition of embodiment 151, wherein the lentiviral vector is selected from the group consisting of: human immunodeficiency virus 1 (HIV-1); human immunodeficiency virus 2 (HIV-2), visna-maedi virus (VMV) virus; caprine arthritis-encephalitis virus (CAEV); equine infectious anemia virus (EIAV); feline immunodeficiency virus (FIV); bovine immune deficiency virus (BIV); and simian immunodeficiency virus (SIV).
153. The composition of embodiment 151 or embodiment 152, wherein the lentiviral vector is derived from human immunodeficiency cirus-1 (HIV-1) or human immunodeficiency virus 2 (HIV-2).
154. The composition of any one of embodiments 151-153, wherein the lentiviral vector is derived from human immunodeficiency cirus-1 (HIV-1).
155. The composition of any one of the preceding embodiments, wherein the viral vector is pseudotyped.
156. The composition of any one of the preceding embodiments, wherein the viral vector is pseudotyped with an envelope protein from a strain of vesicular stomatitis virus.
157. The composition of embodiment 156, wherein the strain of vesicular stomatitis virus is selected from the group consisting of: Indiana, Alagoas, New Jersey, Isfahan, CoCal, Maraba, or Piry.
158. The composition of any one of the preceding embodiments, wherein the viral vector is pseudotyped with a vesicular stomatitis virus G (VSV-G) protein.
159. The composition of any one of the preceding embodiments, wherein the viral vector is pseudotyped with an envelope derived from a Measles envelope protein, a Sindbis envelope protein, Morbillivirus F and H proteins, Sendai F and HN proteins, or Paramyxoviridae F and H proteins.
160. The composition of any one of the preceding embodiments, wherein the viral vector comprises a polynucleotide comprising a transgene.
161. The composition of embodiment 160, wherein the transgene encodes a therapeutic protein.
162. The composition of embodiment 160 or embodiment 161, wherein the transgene or therapeutic protein is a for the treatment of a monogenetic disease, disorder, or condition.
163. The composition of any one of embodiments 160-162, wherein the transgene or therapeutic protein is a chimeric antigen receptor (CAR), a chimeric costimulatory receptor (CCR), an αβ T cell receptor (αβ-TCR), a γδ T cell receptor (γδ-TCR), a dimerizing agent regulated immunoreceptor complex (DARIC), or switch receptor.
164. The composition of any one of embodiments 160-162, wherein the transgene or therapeutic protein is a therapeutic globin for treatment of a hemoglobinopathy or an ABCD1 gene for the treatment of CALD.
165. The composition of any one of the preceding embodiments, wherein the composition does not comprise serum.
166. The composition of any one of the preceding embodiments, wherein the composition does not comprise PIPES.
167. The composition of any one of the preceding embodiments, wherein the composition does not comprise sodium citrate.
168. The composition of any one of the preceding embodiments, wherein the composition does not comprise sodium phosphate.
169. The composition of any one of the preceding embodiments, wherein the composition does not comprise Tris.
170. The composition of any one of the preceding embodiments, wherein the composition does not comprise salt.
171. The composition of any one of the preceding embodiments, wherein the composition does not comprise a chloride salt.
172. The composition of any one of the preceding embodiments, wherein the composition does not comprise NaCl.
173. The composition of any one of the preceding embodiments, wherein the composition does not comprise KCl.
174. The composition of any one of the preceding embodiments, wherein the composition does not comprise trehalose.
175. The composition of any one of the preceding embodiments, wherein the viral vector maintains greater than about 75% infectious titer recovery in HOS cells after storage, relative to the infectious titer of the viral vector in the composition prior to storage or at least one freeze-thaw cycle.
176. The composition of any one of the preceding embodiments, wherein the viral vector maintains greater than about 80% infectious titer recovery in HOS cells after storage, relative to the infectious titer of the viral vector in the composition prior to storage or at least one freeze-thaw cycle.
177. The composition of any one of the preceding embodiments, wherein the viral vector maintains greater than about 85% infectious titer recovery in HOS cells after storage, relative to the infectious titer of the viral vector in the composition prior to storage or at least one freeze-thaw cycle.
178. The composition of any one of the preceding embodiments, wherein the viral vector maintains greater than about 90% infectious titer recovery in HOS cells after storage, relative to the infectious titer of the viral vector in the composition prior to storage or at least one freeze-thaw cycle.
179. The composition of any one of the preceding embodiments, wherein the viral vector maintains greater than about 95% infectious titer recovery in HOS cells after storage, relative to the infectious titer of the viral vector in the composition prior to storage or at least one freeze-thaw cycle; relative to the infectious titer of the viral vector in the composition prior to storage or at least one freeze-thaw cycle.
180. The composition of any one of the preceding embodiments, wherein the viral vector has a thermal unfolding temperature of about 56° to about 62° C. as measured by differential scanning fluorimetry (DSF).
181. The composition of any one of the preceding embodiments, wherein the viral vector has a thermal unfolding temperature of about 58° to about 60° C. as measured by differential scanning fluorimetry (DSF).
182. The composition of any one of the preceding embodiments, wherein the viral vector maintains a hydrodynamic diameter of about 150 nm to about 170 nm as measured by dynamic light scattering (DLS) at 25° C. and a viscosity value of 0.967 centipoise (cP), after storage, relative to hydrodynamic diameter of the viral vector in the composition prior to storage or at least one freeze-thaw cycle.
183. The composition of any one of the preceding embodiments, wherein the viral vector maintains at least about 78% potency as measured by transgene expression in PBMCs compared to a reference standard, after storage
184. The composition of any one of the preceding embodiments, wherein the viral vector maintains at least about 80% potency as measured by transgene expression in PBMCs compared to a reference standard, after storage.
185. The composition of any one of the preceding embodiments, wherein the viral vector maintains at least about 85% potency as measured by transgene expression in PBMCs compared to a reference standard, after storage.
186. The composition of any one of the preceding embodiments, wherein the viral vector maintains at least about 90% potency as measured by transgene expression in PBMCs compared to a reference standard, after storage.
187. The composition of any one of the preceding embodiments, wherein the viral vector maintains at least about 95% potency as measured by transgene expression in PBMCs compared to a reference standard, after storage.
188. The composition of any one of the preceding embodiments, wherein there are no visible fiber particles (wispy fibers) after storage.
189. The composition of any one of the preceding embodiments, wherein there are 5 or fewer visible particles (or specs) after storage.
190. The composition of any one of the preceding embodiments, wherein there are 4 or fewer visible particles (or specs) after storage.
191. The composition of any one of the preceding embodiments, wherein there are 3 or fewer visible particles (or specs) after storage.
192. The composition of any one of the preceding embodiments, wherein there are 2 or fewer visible particles (or specs) after storage.
193. The composition of any one of the preceding embodiments, wherein there are 1 or fewer visible particles (or specs) after storage.
194. The composition of any one of the preceding embodiments, wherein there are no visible particles in the composition after storage.
195. The composition of any one of the preceding embodiments, wherein there are no visible fiber particles (wispy fibers) per 50 ml of composition after storage.
196. The composition of any one of the preceding embodiments, wherein there are 5 or fewer visible particles (or specs) per 50 ml of composition after storage.
197. The composition of any one of the preceding embodiments, wherein there are 4 or fewer visible particles (or specs) per 50 ml of composition after storage.
198. The composition of any one of the preceding embodiments, wherein there are 3 or fewer visible particles (or specs) per 50 ml of composition after storage.
199. The composition of any one of the preceding embodiments, wherein there are 2 or fewer visible particles (or specs) per 50 ml of composition after storage.
200. The composition of any one of the preceding embodiments, wherein there are 1 or fewer visible particles (or specs) per 50 ml of composition after storage.
201. The composition of any one of the preceding embodiments, wherein there are no visible particles per 50 ml of composition after storage.
202. The composition of any one of the preceding embodiments, wherein there are no visible fiber particles (wispy fibers) per 36 ml of composition after storage.
203. The composition of any one of the preceding embodiments, wherein there are 5 or fewer visible particles (or specs) per 36 ml of composition after storage.
204. The composition of any one of the preceding embodiments, wherein there are 4 or fewer visible particles (or specs) per 36 ml of composition after storage.
205. The composition of any one of the preceding embodiments, wherein there are 3 or fewer visible particles (or specs) per 36 ml of composition after storage.
206. The composition of any one of the preceding embodiments, wherein there are 2 or fewer visible particles (or specs) per 36 ml of composition after storage.
207. The composition of any one of the preceding embodiments, wherein there are 1 or fewer visible particles (or specs) per 36 ml of composition after storage.
208. The composition of any one of the preceding embodiments, wherein there are no visible particles per 36 ml of composition after storage.
209. The composition of any one of embodiments 175-208, wherein the storage is at 25° C., 2-8° C., or 37° C.
210. The composition of any one of embodiments 175-209, wherein the storage is for 24 hours, 48 hours, 72 hours, 96 hours, 120 hours, 144 hours, 168 hours, or more.
211. The composition of any one of embodiments 175-210, wherein the storage comprises at least 1 freeze-thaw cycle.
212. The composition of embodiment 211, wherein the at least 1 freeze-thaw cycle is 1 freeze thaw cycle.
213. The composition of embodiment 211, wherein the at least 1 freeze-thaw cycle is 2 freeze thaw cycles.
214. The composition of embodiment 211, wherein the at least 1 freeze-thaw cycle is 3 freeze thaw cycles.
215. The composition of embodiment 211, wherein the at least 1 freeze-thaw cycle is 4 freeze thaw cycles.
216. The composition of embodiment 211, wherein the at least 1 freeze-thaw cycle is 5 freeze thaw cycles.
217. The composition of any one of embodiments 211-216, wherein the one or more freeze-thaw cycles comprise freezing the composition at about −65° C. or less for about 1.5 hours or more, and thawing at 30° C. for 1.5 hours.
218. The composition of any one of the preceding embodiments, wherein the composition is frozen.
219. A method for storing a viral vector comprising:
   a) providing the composition according to any one of embodiments 1-218, and
   b) storing the viral composition at a temperature of about 25° C. or lower.
220. A method for storing a viral vector comprising:
   a) providing the composition according to any one of embodiments 1-218, and
   b) storing the viral composition at a temperature of about 2-8° C. or lower.
221. A method for cryopreserving a viral vector comprising:
   a) providing the composition according to any one of embodiments 1-218,
   b) freezing the viral composition, and
   c) storing the viral composition at a temperature of about 0° C. or lower.
222. The method of any one of embodiments 219-221, wherein the method comprises storing the viral composition for at least about 24 hours.
223. The method of any one of embodiments 219-222, wherein the method comprises storing the viral composition for at least about 48 hours.
224. The method of any one of embodiments 219-223, wherein the method comprises storing the viral composition for at least about 72 hours.
225. The method of any one of embodiments 219-224, wherein the method comprises storing the viral composition for at least about 96 hours.
226. The method of any one of embodiments 219-225, wherein the method comprises storing the viral composition for at least about 120 hours.
227. The method of any one of embodiments 219-226, wherein the method comprises storing the viral composition for at least about 148 hours.
228. The method of any one of embodiments 219-227, wherein the method comprises storing the viral composition for at least about 168 hours.
229. A method of expressing a transgene in a cell comprising contacting a cell with the composition of any one of embodiments 1-217.
230. The method of embodiment 229, wherein the cell is a mammalian cell.
231. The method of embodiment 229 or embodiment 230, wherein the cell is a hematopoietic cell.
232. The method of any one of embodiments 229-231, wherein the cell is an immune effector cell.
233. The method of any one of embodiments 229-232, wherein the cell is a CD3⁺, CD4⁺, and/or CD8⁺ cell.
234. The method of any one of embodiments 229-233, wherein the cell is a T cell.
235. The method of any one of embodiments 229-234, wherein the cell is an αβ T cell.
236. The method of any one of embodiments 229-234, wherein the cell is a γδ T cell.
237. The method of any one of embodiments 229-234, wherein the cell is a cytotoxic T lymphocyte (CTL), a tumor infiltrating lymphocyte (TIL), or a helper T cell.
238. The method of any one of embodiments 229-234, wherein the cell is a natural killer (NK) cell or natural killer T (NKT) cell.
239. The method of any one of embodiments 229-231, wherein the cell is a hematopoietic stem or progenitor cell.
240. The method of any one of embodiments 229-231, the cell is a human CD34⁺ hematopoietic stem or progenitor cell.

In general, in the following claims, the terms used should not be construed to limit the claims to the specific embodiments contemplated in the specification and the claims, but should be construed to include all possible embodiments along with the full scope of equivalents to which such claims are entitled. Accordingly, the claims are not limited by the disclosure.

What is claimed is:

1. An aqueous viral composition comprising:
a) a lentiviral vector
b) a HEPES or L-Histidine buffer;
c) sucrose; and
d) L-Proline.

2. The composition of claim 1, wherein the buffer is present at a concentration of about 25 mM to about 30 mM.

3. The composition of any one of the preceding claims, wherein the buffer is present at a concentration of about 27.5 mM.

4. The composition of any one of the preceding claims, wherein the buffer is a HEPES buffer.

5. The composition of any one of the preceding claims, wherein the buffer is an L-Histidine buffer.

6. The composition of any one of the preceding claims, wherein the sucrose is present at a concentration of about 66 mM to about 80 mM.

7. The composition of any one of the preceding claims, wherein the sucrose is present at a concentration of about 73 mM.

8. The composition of any one of claims 1-6, wherein the sucrose is present at a concentration of about 2.0% to about 3.0% by weight per volume of the composition.

9. The composition of any one of claims 1-6, wherein the sucrose is present at a concentration of about 2.5% by weight per volume of the composition.

10. The composition of any one of the preceding claims, wherein the L-Proline is present at a concentration of about 40 mM to about 60 mM.

11. The composition of any one of the preceding claims, wherein the L-Proline is present at a concentration of about 50 mM.

12. The composition of any one of the preceding claims, wherein the composition further comprises a salt.

13. The composition of claim 12, wherein the salt is present at a concentration of about 65 mM to about 85 mM.

14. The composition of claim 13, wherein the salt is present at a concentration of about 75 mM.

15. The composition of any one of claims 12-14, wherein the salt is a chloride salt, KCl, or NaCl.

16. The composition of any one of the preceding claims, wherein the composition further comprises a poloxamer 188.

17. The composition of claim 16, wherein the poloxamer 188 is present at a concentration of about 0.01 mg/ml to about 1 mg/ml.

18. The composition of claim 17, wherein the poloxamer 188 is present at a concentration of about 0.1 mg/ml or about 0.3 mg/ml.

19. The composition of any one of the preceding claims, wherein the composition comprises a pH of about 6.5 to about 8.

20. An aqueous viral composition comprising:
a) a lentiviral vector,
b) about 27.5 mM HEPES,
c) about 73 mM sucrose or about 2.5% sucrose by weight per volume of composition, and
d) about 50 mM L-Proline; wherein the composition comprises a pH of about 7.

21. An aqueous viral composition comprising:
a) a lentiviral vector,
b) about 27.5 mM HEPES,
c) about 73 mM sucrose or about 2.5% sucrose by weight per volume of composition,
d) about 50 mM L-Proline, and
e) about 0.1 mg/mL to about 0.8 mg/ml poloxamer 188;
wherein the composition comprises a pH of about 7.

22. An aqueous viral composition comprising:
a) a lentiviral vector,
b) about 27.5 mM HEPES,
c) about 73 mM sucrose or about 2.5% sucrose by weight per volume of composition,
d) about 50 mM L-Proline, and
e) about 0.3 mg/mL poloxamer 188;
wherein the composition comprises a pH of about 7.

23. An aqueous viral composition comprising:
a) a lentiviral vector,
b) a HEPES buffer,
c) sucrose,
d) a salt; and
e) a poloxamer.

24. The composition of claim 23, wherein the composition does not comprise L-Proline.

25. The composition of claim 23 or claim 24, wherein the HEPES buffer is present at a concentration of about 25 mM to about 30 mM.

26. The composition of any one of claims 23-25, wherein the HEPES buffer is present at a concentration of about 27.5 mM.

27. The composition of any one of claims 23-26, wherein the sucrose is present at a concentration of about 66 mM to about 80 mM.

28. The composition of any one of claims 23-27, wherein the sucrose is present at a concentration of about 73 mM.

29. The composition of any one of claims 23-28, wherein the sucrose is present at a concentration of about 2.0% to about 3.0% by weight per volume of the composition.

30. The composition of any one of claims 23-29, wherein the sucrose is present at a concentration of about 2.5% by weight per volume of the composition.

31. The composition of any one of claims 23-30, wherein the salt is present at a concentration of about 65 mM to about 85 mM.

32. The composition of any one of claims 23-31, wherein the salt is present at a concentration of about 75 mM.

33. The composition of any one of claims 23-32, wherein the salt is KCl or NaCl.

34. The composition of any one of claims 23-33, wherein the poloxamer is present at a concentration of about 0.01 mg/ml to about 1 mg/ml.

35. The composition of any one of claims 23-34, wherein the poloxamer is present at a concentration of about 0.1 mg/ml.

36. The composition of any one of claims 23-35, wherein the poloxamer is poloxamer 188 (P188).

37. The composition of any one of claims 23-36, wherein the composition comprises a pH of about 7 to about 8.

38. An aqueous viral composition comprising:
a) a lentiviral vector,
b) about 27.5 mM HEPES,
c) about 73 mM sucrose by weight per volume of composition,
d) about 75 mM NaCl, and
e) about 0.1 to about 0.8 mg/ml poloxamer 188;
wherein the composition comprises a pH of about 7.

39. The composition of any one of the preceding claims, wherein the viral lentivector is present at a titer from about 1×10⁸ to about 2×10⁹ TU/ml.

40. The composition of any one of the preceding claims, wherein the lentiviral vector is selected from the group consisting of: human immunodeficiency virus 1 (HIV-1); human immunodeficiency virus 2 (HIV-2), visna-maedi virus (VMV) virus; caprine arthritis-encephalitis virus (CAEV); equine infectious anemia virus (EIAV); feline immunodeficiency virus (FIV); bovine immune deficiency virus (BIV); and simian immunodeficiency virus (SIV).

41. The composition of any one of the preceding claims, wherein the lentiviral vector is derived from human immunodeficiency virus-1 (HIV-1) or human immunodeficiency virus 2 (HIV-2).

42. The composition of any one of the preceding claims, wherein the lentiviral vector is derived from human immunodeficiency virus-1 (HIV-1).

43. The composition of any one of the preceding claims, wherein the lentiviral vector is pseudotyped.

44. The composition of any one of the preceding claims, wherein the lentiviral vector is pseudotyped with an envelope protein from a strain of vesicular stomatitis virus.

45. The composition of claim 44, wherein the strain of vesicular stomatitis virus is selected from the group consisting of: Indiana, Alagoas, New Jersey, Isfahan, CoCal, Maraba, or Piry.

46. The composition of any one of the preceding claims, wherein the lentiviral vector is pseudotyped with a vesicular stomatitis virus G (VSV-G) protein.

47. The composition of any one of the preceding claims, wherein the lentiviral vector is pseudotyped with an envelope derived from a Measles envelope protein, a Sindbis envelope protein, Morbillivirus F and H proteins, Sendai F and HN proteins, or Paramyxoviridae F and H proteins.

48. The composition of any one of the preceding claims, wherein the lentiviral vector comprises a polynucleotide comprising a transgene.

49. The composition of claim 48, wherein the transgene encodes a therapeutic protein.

50. The composition of claim 48 or claim 49, wherein the transgene or therapeutic protein is for the treatment of a monogenetic disease, disorder, or condition.

51. The composition of any one of claims 48-50, wherein the transgene or therapeutic protein is a chimeric antigen receptor (CAR), a chimeric costimulatory receptor (CCR), an αβ T cell receptor (αβ-TCR), a γδ T cell receptor (γδ-TCR), a dimerizing agent regulated immunoreceptor complex (DARIC), or switch receptor.

52. The composition of any one of claims 48-51, wherein the transgene or therapeutic protein is a therapeutic globin for treatment of a hemoglobinopathy or an ABCD1 gene for the treatment of CALD.

53. The composition of any one of the preceding claims, wherein the composition does not comprise serum.

54. The composition of any one of the preceding claims, wherein the composition does not comprise PIPES.

55. The composition of any one of the preceding claims, wherein the composition does not comprise sodium citrate.

56. The composition of any one of the preceding claims, wherein the composition does not comprise sodium phosphate.

57. The composition of any one of the preceding claims, wherein the composition does not comprise Tris.

58. The composition of any one of the preceding claims, wherein the composition does not comprise salt.

59. The composition of any one of the preceding claims, wherein the composition does not comprise a chloride salt.

60. The composition of any one of the preceding claims, wherein the composition does not comprise NaCl.

61. The composition of any one of the preceding claims, wherein the composition does not comprise KCl.

62. The composition of any one of the preceding claims, wherein the composition does not comprise trehalose.

63. The composition of any one of the preceding claims, wherein the lentiviral vector maintains greater than about 75% infectious titer recovery in HOS cells after storage, relative to the infectious titer of the lentiviral vector in the composition prior to storage or at least one freeze-thaw cycle.

64. The composition of any one of the preceding claims, wherein the lentiviral vector maintains greater than about 95% infectious titer recovery in HOS cells after storage, relative to the infectious titer of the lentiviral vector in the composition prior to storage or at least one freeze-thaw cycle; relative to the infectious titer of the lentiviral vector in the composition prior to storage or at least one freeze-thaw cycle.

65. The composition of any one of the preceding claims, wherein the lentiviral vector has a thermal unfolding temperature of about 56° to about 62° C. as measured by differential scanning fluorimetry (DSF).

66. The composition of any one of the preceding claims, wherein the lentiviral vector maintains a hydrodynamic diameter of about 150 nm to about 170 nm as measured by dynamic light scattering (DLS) at 25° C. and a viscosity value of 0.967 centipoise (cP), after storage, relative to hydrodynamic diameter of the lentiviral vector in the composition prior to storage or at least one freeze-thaw cycle.

67. The composition of any one of the preceding claims, wherein the lentiviral vector maintains at least about 78% potency as measured by transgene expression in PBMCs compared to a reference standard, after storage

68. The composition of any one of the preceding claims, wherein the lentiviral vector maintains at least about 95% potency as measured by transgene expression in PBMCs compared to a reference standard, after storage.

69. The composition of any one of the preceding claims, wherein there are no visible fiber particles (wispy fibers) after storage.

70. The composition of any one of the preceding claims, wherein there are 5 or fewer visible particles or specs after storage.

71. The composition of any one of the preceding claims, wherein there are no visible particles in the composition after storage.

72. The composition of any one of claims 63-71, wherein the storage is at 25° C., 2-8° C., or 37° C.

73. The composition of any one of claims 63-72, wherein the storage is for 24 hours, 48 hours, 72 hours, 96 hours, 120 hours, 144 hours, 168 hours, or more.

74. The composition of any one of claims 63-73, wherein the storage comprises one or more freeze-thaw cycles.

75. The composition of any claim 74, wherein the one or more freeze-thaw cycle comprise freezing the composition at about −65° C. or less for about 1.5 hours or more, and thawing at 30° C. for 1.5 hours.

76. The composition of any one of the preceding claims, wherein the composition is frozen.

77. A method for storing a viral vector comprising:
a) providing the composition according to any one of claims 1-76, and
b) storing the composition at a temperature of about 25° C. or lower.

78. A method for storing a viral vector comprising:
a) providing the composition according to any one of claims 1-76, and
b) storing the composition at a temperature of about 2-8° C. or lower.

79. A method for cryopreserving a viral vector comprising:
a) providing the composition according to any one of claims 1-76,
b) freezing the composition, and
c) storing the composition at a temperature of about 0° C. or lower.

80. The method of any one of claims 77-79, wherein the method comprises storing the lentiviral composition for at least about 24 hours.

81. The method of any one of claims 77-79, wherein the method comprises storing the lentiviral composition for at least about 168 hours.

82. A method of expressing a transgene in a cell comprising contacting a cell with the composition of any one of claims 1-76.

83. The method of claim 82, wherein the cell is a mammalian cell.

84. The method of claim 82 or claim 83, wherein the cell is a hematopoietic cell.

85. The method of any one of claims 82-84, wherein the cell is an immune effector cell.

86. The method of any one of claims 82-85, wherein the cell is a CD3⁺, CD4⁺, and/or CD8⁺ cell.

87. The method of any one of claims 82-86, wherein the cell is a T cell.

88. The method of any one of claims 82-87, wherein the cell is an αβ T cell.

89. The method of any one of claims 82-87, wherein the cell is a γδ T cell.

90. The method of any one of claims 82-87, wherein the cell is a cytotoxic T lymphocyte (CTL), a tumor infiltrating lymphocyte (TIL), or a helper T cell.

91. The method of any one of claims 82-85, wherein the cell is a natural killer (NK) cell or natural killer T (NKT) cell.

92. The method of any one of claims 82-85, wherein the cell is a hematopoietic stem or progenitor cell.

93. The method of any one of claims 82-85, the cell is a human CD34+ hematopoietic stem or progenitor cell.
